(19) United States
(12) Patent Application Publication
(10) Pub. No.: US 2024/0219302 A1
(43) Pub. Date: Jul. 4, 2024

(54) OPTICAL EMISSION BIOSENSING USING MAGNETIC BEADS WITH A FAST AGGREGATION TIME

(71) Applicant: Bar-Ilan University, Ramat-Gan (IL)

(72) Inventors: Amos DANIELLI, Moshav Hadar-Am (IL); Shmuel BURG, Herzliya (IL)

(73) Assignee: Bar-Ilan University, Ramat-Gan (IL)

(21) Appl. No.: 18/289,130

(22) PCT Filed: May 3, 2022
(86) PCT No.: PCT/IL2022/050462

§ 371 Date: Nov. 1, 2023

Related U.S. Application Data
(60) Provisional application No. 63/183,087, filed on May 3, 2021.

(51) Int. Cl. G01N 21/64; G01N 21/21; G01N 21/27

(52) U.S. Cl. CPC G01N 21/6428; G01N 21/274; G01N 21/645; G01N 2021/218

(57) ABSTRACT

An assay method for target molecules in a sample using optical emission from magnetic beads, comprising:
a) preparing the magnetic beads so, if excited, they produce optical emission as a consequence of contact between the beads, reporter molecules and target molecules in the sample;
b) providing the prepared magnetic beads in a solution in a container, with one or more magnets producing a magnetic field inside the container that causes the beads to aggregate into a clump inside the container in less than 30 seconds;
c) exciting the optical emission from the magnetic beads in the clump; and
d) measuring the optical emission from the magnetic beads in the clump.

### RELATED APPLICATION

This application claims the benefit of priority of U.S. Provisional Patent Application No. 63/183,087 filed on 3 May 2021, the contents of which are incorporated herein by reference in their entirety.

### FIELD AND BACKGROUND OF THE INVENTION

The present invention, in some embodiments thereof, relates to bioassays and, more particularly, but not exclusively, to bioassays using fluorescent reporter molecules and magnetic beads.

International Patent Application Publication number WO 2018/185672, “Bio-Assay Capture Surfaces with Bleached Autofluorescence,” to MagBiosense, Inc., with inventors Amos Danielli, Or Hadass, and Jasenka Verbarg, describes “magnetic modulation biosensing (MMB) methods” for performing bio-assays, and states that, “In this type of bio-assay, a magnetic field aggregates the beads, and varying the gradient of the magnetic field over time causes the position of the aggregated beads to vary in time, passing into and out of the beam of the excitation light, for example periodically. At the same time, a component of the bio-assay signal, that varies in time synchronously with the magnetic field gradient, is measured. This type of bio-assay provides particularly high sensitivity, because it makes it possible to distinguish the fluorescent signal emitted by the beads, which is modulated over time, from a background signal coming from the surrounding solution, for example from Raman scattering from the surrounding water molecules, and from any unbound fluorescent molecules in the solution, which is not modulated over time.” It also states that, “Alternatively, instead of or in addition to moving magnetic clump 212 into and out of light beam 216, light beam 216 is moved so that it is alternately aimed at magnetic clump 212, and aimed to the side of magnetic clump 212. This can be done even without moving magnetic clump 212, and it can have the same effect as moving clump 212, that clump 212 alternately is illuminated and not illuminated by light beam 216 . . . Moving light beam 216, instead of or in addition to moving clump 212, has the potential advantage that it might be possible to do more rapidly than moving clump 212, for example at 10 Hz rather than 2 Hz, thereby modulating the illumination of clump 212 more rapidly.” The application also states that, “ . . . magnets 204 and 206 are . . . located to the sides of container 202, though the orientation of system 200 with respect to gravity is relatively unimportant if the magnetic force on the beads is much greater than the force of gravity on the beads. Magnet 204 has a small sharply curved pole piece 208, adjacent to container 202 on one side, and magnet 206 has a similar pole piece 210, adjacent to container 202 on the other side. Each pole piece, when its associated electromagnet is turned on, produces a high enough magnetic field in the container to magnetize the beads and make them clump together.”

Shmuel Burg, Meir Cohen, Michael Margulis, Shira Roth, and Amos Danielli, “Magnetically aggregated biosensors for sensitive detection of biomarkers at low concentrations,” Appl. Phys. Lett. 115, 103702 (2019), <www(dot)doi(dot)org/10(dot)1063/1(dot)5108891>, proposes “a compact fluorescence-based system that simply uses a small permanent magnet with a conic tip to aggregate the magnetic beads, forming a cluster of fluorescently labeled probes whose fluorescence signal is much greater than that of a single bead. Using the magnetically aggregated biosensors to detect human Interleukin-8, we demonstrated a limit of detection of 0.1 ng/l and a 4-log dynamic range performance, which is on par with the most sensitive devices but is achieved without their bulk and cost.”

Additional background art includes Danielli, A., Aric, A., Porat, N. and Ehrlich, M., “Detection of fluorescent-labeled probes at subpicomolar concentrations by magnetic modulation,” Optics Express 16, 19253-19259 (2008); Danielli, A., Porat, N., Aric, A. and Ehrlich, M., “Rapid homogenous detection of the Ibaraki virus NS3 cDNA at picomolar concentrations by magnetic modulation,” Biosensors & Bioelectronics 25, 858-863, doi: 10.1016/j.bios.2009.08.047 (2009); Verbarg, J., Hadass, O., Olivo, P. D. and Danielli, A., “High sensitivity detection of a protein biomarker Interleukin-8 utilizing a magnetic modulation biosensing system,” Sensor and Actuators B: Chemical 241, 614-618 (2017); M Margulis and A. Danielli, “Rapid and sensitive detection of repetitive nucleic acid sequences using magnetically modulated biosensors,” ACS Omega 4, 11749-11755 (2019); J. Verbarg, O. Hadass, P. D. Olivo, and A. Danielli, “High sensitivity detection of a protein biomarker Interleukin-8 utilizing a magnetic modulation biosensing system,” Sensors and Actuators B: Chemical 241, 614-618 (2017); and M Margulis, S. Ashri, M. Cohen and A. Danielli, “Detecting nucleic acid fragments in serum using a magnetically modulated sandwich assay,” Journal of Biophotonics, e201900104 (2019).

### SUMMARY OF THE INVENTION

An aspect of some embodiments of the invention concerns a bioassay using optical emission from magnetic beads in a container, where the magnetic beads are aggregated into a clump at the bottom of the container, by a magnet located just below the bottom of the container, before the optical emission is measured; and/or a photobleaching rate of the beads is measured and taken into account; and/or a background level of the optical emission is measured before the beads begin to aggregate; and/or the beads are held in place on an inner surface of the container by a magnet just outside the container while fluid flows past the beads, washing away molecules that produce stray optical emission.

There is thus provided, in accordance with an exemplary embodiment of the invention, an assay method for target molecules in a sample is using as a consequence of contact between the beads, reporter molecules and target molecules in the sample optical emission from magnetic beads, comprising:
a) preparing the magnetic beads so, if excited, they produce optical emission;
b) providing the prepared magnetic beads in a solution in a container, with one or more magnets producing a magnetic field inside the container that causes the beads to aggregate into a clump inside the container in less than 30 seconds;
c) exciting the optical emission from the magnetic beads in the clump; and
d) measuring the optical emission from the magnetic beads in the clump.

Optionally, the one or more magnets comprise at least one permanent magnet.

Optionally, the permanent magnet comprises a material with energy product greater than 100 kilojoules per cubic meter.

Optionally, the magnetic field inside the container changes by less than 10% in amplitude and less than 0.1 radians in direction during a time interval when the beads are aggregating before the optical emission is measured, and when the optical emission is measured.

Optionally, the clump covers at least one contiguous area of an inner surface of the container, covered by the solution, that extends at least 0.1 mm in all directions along the inner surface, and the beads are densely enough packed in the clump to cover at least 60% of the inner surface in that area.

Optionally, the contiguous area is less than 10% of an area of the inner surface of the container.

Optionally, the one or more magnets producing the magnetic field are located beneath the container.

Optionally, the one or more magnets have a magnetization oriented substantially in a same vertical direction.

Optionally, the magnetic field causes the beads to aggregate into the clump at a location on a bottom surface of the inside of the container.

Optionally, the solution has a depth of less than 4 mm above the location on the bottom surface where the clump aggregates.

Optionally, a volume of the solution in the container is wider in all horizontal directions than it is deep vertically.

Optionally, exciting the optical emission comprises illuminating the magnetic beads in the clump with an excitation light beam.

In an exemplary embodiment of the invention, illuminating the magnetic beads in the clump comprises passing the excitation light beam through a first volume of the solution on the way to the clump, and measuring the optical emission from the magnetic beads in the clump comprises:
a) measuring an optical emission signal from the beads together with a background signal from the first volume;
b) illuminating a second volume of the buffer solution substantially without illuminating the clump, with the same or a substantially similar excitation light beam, and measuring a background signal from the second volume; and
c) determining the optical emission from the magnetic beads, using the emission signal from the magnetic beads plus the background signal from the first volume, and the background signal from the second volume.

Optionally, illuminating the second volume comprises measuring the optical emission signal from the beads together with the background signal from the first volume, before or after measuring the background signal from the second volume with the same excitation light beam, redirecting the excitation light beam from one to the other.

Optionally, the method also comprises:
a) repeatedly directing the same excitation light beam to alternately illuminate the clump through the first volume, and the second volume substantially without illuminating the clump;
b) measuring the emission signal of the beads together with the background signal from the first volume when the excitation light beam is illuminating the clump through the first volume; and
c) measuring the background signal from the second volume when the excitation light beam is illuminating the second volume.

Alternatively, illuminating the clump through the first volume, and illuminating the second volume, are done with two different light beams, respectively a first beam and a second beam, that originate from two different light sources, or that are split off by a beam splitter from a single beam.

Optionally, the first and second beams are alternately switched on and off, one or more times, by opening and closing shutters, or, if the first and second beams originate from two different light sources, by alternately switching the two different light sources on and off.

Optionally, the first beam illuminates the clump through the first volume, and the second beam illuminates the second volume, simultaneously, and measuring the emission signal from the magnetic beads together with the background signal from the first volume is done simultaneously with measuring the background signal from the second volume.

Optionally, the second volume is substantially equal in volume to the first volume, the light beam illuminating the clump through the first volume has substantially the same power and spectrum as the light beam illuminating the second volume, and determining the optical emission from the magnetic beads comprises subtracting the background signal from the second volume, from the emission signal from the magnetic beads plus the background signal from the first background.

Optionally, the excitation light beam or beams illuminate the clump through the first volume, and illuminate the second volume substantially vertically from above, through the solution.

Optionally, the solution is less than 5 mm deep above the clump.

Optionally, measuring the optical emission from the magnetic beads plus the background emission from the first volume, and measuring the background emission from the second volume, are both done with a same light sensing device.

Optionally, the light power of the excitation light beam, at the clump, is concentrated narrowly enough so that at least 70% of the light power illuminates the clump.

Optionally, the optical emission comprises fluorescent emission, and the excitation light beam excites the fluorescent emission.

Optionally, illuminating the beads comprises performing a plurality of cycles of illuminating the beads with the excitation light beam for a first time interval, then not illuminating the beads with the excitation light beam for a second time interval, wherein the second time interval is long enough so that the beads recover at least 80% of a reduction in the emission coefficient due to photobleaching that occurred during the first time interval.

Optionally, measuring the emission comprises measuring the emission as a function of time, the method also comprising determining a rate of decrease of the emission coefficient due to the photobleaching, from the measured emission as a function of time.

Optionally, illuminating the beads comprising illuminating the beads during a first time interval, the method also comprising determining a concentration of the target molecules in the sample from the measured emission.

Optionally, the method also includes not illuminating the beads with the excitation light beam during a second time interval following the first time interval, long enough for the emission coefficient to recover at least 80% of its decrease during the first time interval, and repeating, at least once, a cycle of photobleaching by illuminating the beads and recovery of the emission coefficient by not illuminating the beads, while measuring the emission as a function of time.

Optionally, any net decrease in the emission coefficient following the photobleaching and recovery in each cycle is less than 5%.

Optionally, the one or more magnets producing the magnetic field are located beneath the container, and the magnetic field causes the beads to aggregate into the clump at a location on a bottom surface of the inside of the container.

Optionally, the method comprises illuminating the location on the bottom surface with the excitation light beam, and measuring light coming from the location in a range of wavelengths of the fluorescent emission, starting before the beads begin to aggregate into the clump, wherein performing the cycles of photobleaching and recovery while measuring the emission as a function of time is done during a time period of the beads aggregating into the clump.

Optionally, determining the concentration of target molecules from the measured emission comprises using the measurement of light coming from the location in the range of fluorescent emission wavelengths to find a background level of the fluorescent emission, and correcting the emission measured after the beads start aggregating, for the background level.

Optionally, determining the concentration of target molecules from the measured emission comprises determining a rate of photobleaching from the measured emission as a function of time during the first time intervals of the cycles, and correcting the measured emission for the photobleaching.

Optionally, performing the cycle of photobleaching and recovery while measuring the emission as a function of time continues until the beads are fully aggregated into the clump, and determining the concentration of target molecules from the measured emission comprises using a level of emission measured when the beads are fully aggregated.

Optionally, determining the concentration of target molecules from the measured emission comprises using a rate of increase in the level of emission during at least a portion of the time period of the beads aggregating into the clump.

Optionally, determining the concentration of target molecules from the measured emission comprises using the rate of increase in the level of emission during a portion of the time period when the level of emission is at least 70% of a peak value of the level of emission.

Optionally, determining the concentration of target molecules from the measured emission comprises using the rate of increase in the level of emission during a portion of the time period when the level of emission is less than 70% of a peak value of the level of emission.

Optionally, determining the concentration of target molecules from the measured emission comprises using both the level of optical emission, and the rate of decrease of the emission coefficient due to the photobleaching, taking into account a dependence of the rate of photobleaching on the concentration of target molecules.

Optionally, the method comprises illuminating the location on the bottom surface with the excitation light beam, and measuring light coming from the location in a range of wavelengths of the optical emission, starting before the beads begin to aggregate into the clump, and exciting the optical emission from the magnetic beads in the clump comprises illuminating the location with the excitation light beam while the beads are aggregating, and measuring the optical emission from the magnetic beads in the clump comprises measuring the emission from the location as a function of time while the beads are aggregating.

Optionally, the method comprises determining a concentration of target molecules in the sample from the measured emission by using the measurement of light coming from the location in the range of optical emission wavelengths to find a background level of the optical emission, and correcting the emission measured after the beads start aggregating for the background level.

Optionally, measuring the emission as a function of time continues until the beads are fully aggregated into the clump, and determining the concentration of target molecules from the measured emission comprises using a level of emission measured when the beads are fully aggregated.

Optionally, the method comprises determining a concentration of target molecules in the sample from the measured emission using a rate of increase in the level of emission during a time interval when the level of emission has not yet reached its maximum value.

Alternatively or additionally, the optical emission comprises chemiluminescence, and exciting the optical emission comprises exposing the magnetic beads to a chemical that excites the chemiluminescence.

Alternatively or additionally, the optical emission comprises electro-chemical luminescence, and exciting the optical emission comprises passing an excitation current through the magnetic beads.

Optionally, preparing the magnetic beads comprises preparing the beads so that they produce the optical emission at a level that depends on the concentration, quantity, or both, of target molecules in the sample.

Optionally, the method also comprises determining the concentration, quantity, or both, of the target molecules in the sample from the measured optical emission.

Optionally, preparing the magnetic beads comprises preparing the magnetic beads according to a sandwich assay.

Alternatively, preparing the magnetic beads comprising preparing the magnetic beads according to an energy transfer assay.

Optionally, the one or more magnets comprise at least one magnet that has a tip with a sharp point at its end in a direction of magnetization.

Optionally, the magnet with a sharp tip is located outside the container, and the sharp point is at a part of the magnet's surface that is closest to the container.

Optionally, a dot product of a magnetic field B produced by the magnet, and magnetic field gradient VB produced by the magnet, is greater than 0.2 T²/m over at least 50% of the solution in the container.

Optionally, for most of the magnetic beads, a travel time of the magnetic bead from anywhere in at least 50% of the solution in the container to a location at an inner surface of the container where the magnetic field is greatest, is less than 20 seconds, if the bead were to travel at an instantaneous velocity for which a magnetic force on the bead by the magnetic field balances a drag force on the bead in water at 20° C., at each location that the bead passes.

Optionally, measuring the optical emission from the magnetic beads is done with a camera.

Optionally, the target molecules are DNA or RNA molecules of, or including, a specified nucleotide sequence.

Optionally, the clump has a diameter between 0.1 mm and 1 mm.

Optionally, the method comprises performing the assay method according to exemplary embodiment of the invention a plurality of times successively using different samples, using different wells of a same well plate for the container each time, using the same one or more magnets each time, and positioning the well plate each time so that the well being used for that assay is over the one or more magnets when the assay is performed.

Optionally, providing the prepared magnetic beads in the container comprises placing the magnetic beads into the container when the one or more magnets are already producing the magnetic field inside the container.

Alternatively, providing the prepared magnetic beads in the container comprises positioning the one or more magnets relative to the container to produce the magnetic field inside the container after the magnetic beads are already in the container.

Optionally, the beads form a clump adjacent to the one or more magnets within 20 seconds of an earliest time when the magnetic beads are in the solution in the container, and the one or more magnets are positioned to produce the magnetic field inside the container.

Optionally, the solution comprises one or more of a buffer solution, and a biological fluid that is part of the sample.

In an exemplary embodiment of the invention, preparing the magnetic beads comprises:
a) entering the sample into a microfluidics cartridge;
b) exposing the sample, inside an incubation chamber of the cartridge, to probe molecules that selectively bind to the target molecules;
c) binding at least a portion of each probe molecule that was bound to a target molecule, indirectly to a magnetic bead; and
d) processing the beads, inside the same or another incubation chamber of the cartridge, so that an optical emission from reporter molecules bound to the probe molecules or portions of probe molecules, and also bound to the beads, will depend on how many probe molecules were bound to the target molecules;
and exciting the optical emission and measuring the optical emission are also done in a chamber of the cartridge.

Optionally, exciting the optical emission and measuring the optical emission are done in a detection chamber of the cartridge, the same as or different from the incubation chamber or chambers.

Optionally, the container comprises a channel with the one or more magnets adjacent to it, and providing the prepared magnetic beads in a solution comprises:
a) causing the solution with the beads suspended in it to flow through the channel past the adjacent one or more magnets at a slow enough speed so that the magnetic field traps the beads and causes them to aggregate into the clump on an inner surface of the channel; and
b) causing the solution to continue to flow through the channel past the clump after it has aggregated;
and exciting and measuring the optical emission comprise exciting and measuring the optical emission after the continuing flow of the solution past the aggregated clump has washed away most loose molecules in the solution that would otherwise produce a background level of the optical emission from a vicinity of the clump.

Optionally, the one or more magnets are located beneath the channel, and the inner surface of the channel where the clump aggregates is a bottom surface.

Optionally, the one or more magnets comprise at least a first magnet adjacent to a first location in the channel, and a second magnet adjacent to a second location further along the channel in the direction of flow of the solution, and at least some beads that fail to be trapped and aggregated into a clump in the first location by the first magnet are trapped by second magnet and aggregate into a second clump at the second location, and the method also comprises exciting the optical emission and measuring the optical emission from the magnetic beads in the clump, and from the second clump.

Optionally, measuring the optical emission from the magnetic beads in the clump comprises using a digital camera to acquire an image of the clump, and blurring the image over a distance at least 5 times greater than a bead diameter, and at least 2 times greater than a pixel width in the image, but not greater than 2 times a diameter of the clump.

Optionally, blurring the image comprises moving the container relative to the digital camera when acquiring the image.

Optionally, blurring the image comprises blurring the image by image processing.

There is further provided, in accordance with an exemplary embodiment of the invention, a system for measuring an optical emission signal from a quantity of magnetic beads in an assay of target molecules in a sample, comprising:
a) a container configured for holding the magnetic beads in a volume of a solution;
b) one or more magnets adjacent to the container, the magnets producing a magnetic field with a field gradient in the volume of solution that attracts the beads to form a clump at a bottom inner surface of the container adjacent to at least one of the magnets;
c) detection optics and a light sensing device that receives and measures the optical emission signal from the clump of magnetic beads; and
d) a recording device that outputs and/or stores data of the optical emission signal.

Optionally, the one or more magnets are located below the container, and a dot product of the magnetic field and the field gradient, at a top of the volume of the solution directly above the one or more magnets is at least 0.2 teslas squared per meter.

Optionally, for M280 magnetic beads, a ratio of magnetic force, to gravitational force reduced by buoyant force of the solution, on said magnetic beads, is greater than 10 throughout the volume of solution.

Optionally, the system also comprises a light source and illumination optics configured for illuminating the clump of beads with a beam of fluorescent excitation light focused narrowly on the clump, for an assay where the optical emission comprises fluorescent emission.

Optionally, at least a part of the illumination optics is shared by the detection optics.

Optionally, the illumination optics comprises a light beam deflecting element configured to direct the light beam to illuminate the clump of beads passing through a first volume of the solution, or to pass through a second volume of the solution going to the side of the clump, and wherein the recording device outputs and/or stores data of a background optical signal received from the second volume of the solution when the beam is directed to the side of the clump, in addition to outputting and/or storing data of the optical emission signal when the beam is illuminating the beads through the first volume of the solution.

Optionally, the light beam deflecting element is configured to repeatedly alternate between directing the light beam to illuminate the clump of beads through the first volume, and to pass through the second volume going to the side of the clump.

Alternatively, the system also comprises a second light source that generates a second light beam, or a beam splitter that generates a second light beam from the first light source, wherein the illumination optics that directs and narrowly focuses the first light beam to illuminate the clump of beads through a first volume of the solution and directs and narrowly focuses the second light beam to pass through a second volume of the solution to the side of the clump, and wherein the recording device outputs and/or stores data of a background optical signal received from the second volume of the solution when the beam is directed to the side of the clump, in addition to outputting and/or storing data of the optical emission signal when the beam is illuminating the beads through the first volume of the solution.

Optionally, the system also comprises a beam switching mechanism that blocks or turns off the second beam when the first beam is illuminating the clump through the first volume, and blocks or turns off the first beam when the second beam is illuminating the second volume. Optionally, the beam switching mechanism is configured to repeatedly alternate between first beam illuminating the clump through the first volume, and the second beam illuminating the second volume.

Optionally, the system also comprises a processor configured to use the data of the background signal received from the second volume to correct the data of optical emission from the clump for background emission received from the first volume when the beam is illuminating the clump through the first volume.

Optionally, the light sensing device comprises a camera that distinguishes light emitted from the clump and the first volume, from light emitted from the second volume, by sensing them on different pixels.

Optionally, the system also comprises a current source configured to pass electric current through the clump, for an assay where the optical emission comprises electro-chemical luminescence.

Optionally, the one or more magnets are below and adjacent to a bottom of the container. Optionally, the container is one of a plurality of substantially similar wells comprised in a well plate, each well configured for holding the magnetic beads in the solution, and each well configured, at a same time or at different times, for attracting the beads into a clump at the bottom of the well using a magnetic field gradient, and for detecting an optical emission from the clump at the bottom of each well.

Optionally, the system also comprises a motor or actuator configured for moving the well plate horizontally, relative to the detection optics and the light detecting device, or relative to the illumination optics and light source, or relative to both, successively moving different wells adjacent to the same detection optics and light detecting device, or to the same illumination optics and light source, or to both.

Optionally, the system also comprises a motor or actuator configured for moving the well plate horizontally, relative to the one or more magnets, successively bringing different wells above and adjacent to the same one or more magnets.

Optionally, the motor or actuator also moves the well plate relative to the detection optics and the light detecting device, or relative to the illumination optics and light source, or relative to both, such that, when a well is above and adjacent to the same one or more magnets, it is also adjacent to the same detection optics and light detecting device, or to the same illumination optics and light source, or both.

Optionally, the system also comprises a light source and illumination optics comprising an optical fiber extending from the light source to each well, configured for illuminating the clump of beads with a beam of fluorescent excitation light focused narrowly on the clump in each well, for an assay where the optical emission comprises fluorescent emission.

In an exemplary embodiment of the invention, the system also comprises a microfluidics cartridge that comprises:
a) the container;
b) one or more chambers configured for stably storing the magnetic beads and one or more reagents used for performing the assay, before the assay is performed;
c) an input port for entering the sample into the cartridge; and
d) one or more incubation chambers, the same as or different from the container, configured for performing the assay on the sample after it has been entered into the cartridge, using the reagents and the magnetic beads.

Optionally, the system also comprises a controller that transfers one or more of the sample, the magnetic beads and the reagents between different chambers of the cartridge in order to perform the assay, and that transfers the magnetic beads into the container before they are formed into the clump and before their optical emission signal is measured.

There is further provided, in accordance with an exemplary embodiment of the invention, an assay method for target molecules in a sample measuring optical emission from magnetic beads, comprising:
a) preparing the magnetic beads so, if excited, they produce optical emission as a consequence of contact between the beads, reporter molecules and target molecules in the sample;
b) providing the prepared magnetic beads in a solution in a container, with one or more magnets producing a magnetic field inside the container that causes the beads to aggregate into a clump inside the container;
c) exciting the optical emission from the magnetic beads in the clump by illuminating the clump with an excitation light beam thereby causing both optical emission from the beads and photobleaching of the beads;
d) measuring the optical emission from the magnetic beads in the clump as a function of time; and
e) determining a rate of the photobleaching of the beads, from the measured emission as a function of time within one or more time periods when the clump is illuminated.

Optionally, the method comprises correcting the measured emission for the rate of photobleaching.

Optionally, the method comprises determining a concentration of target molecules in the sample using both the level of optical emission, and the rate of photobleaching, taking into account a dependence of the rate of photobleaching on the concentration of target molecules.

There is further provided, in accordance with an exemplary embodiment of the invention, an assay method for target molecules in a sample measuring optical emission from magnetic beads, comprising:
a) preparing the magnetic beads so, if excited, they produce optical emission as a consequence of contact between the beads, reporter molecules and target molecules in the sample;
b) providing the prepared magnetic beads in a solution in a container, with one or more magnets producing a magnetic field inside the container that causes the beads to aggregate into a clump at a location on an inside surface of the container;
c) illuminating the location with an excitation light beam starting before the clump starts to aggregate;
d) causing the beads to start aggregating into the clump by introducing the beads into the container, bringing the one or more magnets close to the location, or both;
e) exciting the optical emission from the beads by continuing to illuminate the location as the clump aggregates;
f) measuring light at a range of wavelengths of the optical emission from the location starting before the clump starts to aggregate;
g) continuing to measure light at the range of optical emission wavelengths coming from the location, including optical emission from the magnetic beads, as a function of time as the clump aggregates;
h) using the light measured from the location before the clump starts to aggregate to determine a background level of light in the range of optical emission wavelengths coming from the location; and
i) determining a corrected level of optical emission from the clump, by adjusting the measured light at the range of optical emission wavelengths coming from the location after the clump has at least partly aggregated, for the background level.

Optionally, illuminating the location and measuring light from the location continues until the clump is substantially fully aggregated, and the corrected level of optical emission from the clump is determined at least when the clump is substantially fully aggregated.

There is further provided, in accordance with an exemplary embodiment of the invention, an assay method of target molecules in a sample measuring optical emission from magnetic beads, comprising:
a) preparing the magnetic beads so, if excited, they produce optical emission as a consequence of contact between the beads, reporter molecules and target molecules in the sample;
b) causing a solution with the prepared magnetic beads suspended in it to flow through a channel with one or more magnets adjacent to it that produce a magnetic field in the channel that traps one or more of the beads at a location at an inner surface of the channel, while the flow continues;
c) exciting the optical emission from the one of more magnetic beads at the location; and
d) measuring the optical emission from the one or more magnetic beads at the location;
wherein exciting and measuring the optical emission comprise exciting and measuring the optical emission after the continuing flow of the solution past the trapped one or more beads has washed away most loose molecules in the solution that would otherwise produce a background level of the optical emission from the location.

Optionally, the one or more adhering beads comprise at least 10 beads densely packed in an area of the inner surface.

There is further provided, in accordance with an exemplary embodiment of the invention, a system for measuring an optical emission signal from a quantity of magnetic beads in an assay of target molecules in a sample, comprising:
a) a channel at least twice as wide as the magnetic beads;
b) one or more magnets adjacent to the channel, the magnets producing a magnetic field with a field gradient in the channel that attracts the beads when they are in a solution flowing through the channel, and stops them on an inner surface of the channel adjacent to at least one of the magnets, while the solution continues to flow past them; and
c) detection optics that receives and measures the optical emission signal from the stopped magnetic beads in the channel.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWING(S)

Some embodiments of the invention are herein described, by way of example only, with reference to the accompanying drawings and images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 schematically shows a system for performing a bio-assay by measuring optical emission from magnetic beads that are aggregated into a clump at the bottom of a container by a magnet with a sharp tip beneath the bottom of the container, according to an exemplary embodiment of the invention;

FIG. 2 is a flowchart for a method of performing a bio-assay using the system of FIG. 1, according to an exemplary embodiment of the invention;

FIG. 3A schematically shows the magnet, located just below the bottom of the container, aggregating the magnetic beads in the system of FIG. 1, according to an exemplary embodiment of the invention;

FIG. 3B shows an alternative exemplary configuration to that shown in FIG. 3A, in which the magnet is located further away from the bottom of the container than in FIG. 3A, and the beads aggregate into a broader, flatter clump than in FIG. 3A;

FIG. 4 is a plot showing the magnetic field B and the gradient of the magnetic field in the container as a function of distance directly above the tip of the magnet, for an exemplary magnet that could be used in the system of FIG. 1, that was used in tests done by the inventors;

FIG. 5 schematically shows a system for performing a bio-assay by measuring fluorescent emission from magnetic beads that are aggregated into a clump by a magnet beneath the bottom of a container, including a light beam that illuminates the beads for excitation of the fluorescent emission, according to an exemplary embodiment of the invention;

FIG. 6 is a flowchart for a method of performing a bio-assay by measuring fluorescent emission from magnetic beads aggregated into a clump by a magnet beneath the bottom of a container, correcting for background emission, according to an exemplary embodiment of the invention;

FIG. 7 schematically shows a system that can be used to perform a bio-assay according to the method of FIG. 6, in which a rotating mirror causes the excitation light beam to be aimed alternately at the magnetic beads in the clump, and to the side of the clump;

FIG. 8 shows an image showing the fluorescent emission when the excitation light beam of a system like the system of FIG. 7 is aimed at the clump of beads, and an image showing the background emission when the excitation light beam is aimed to the side of the clump of beads;

FIG. 9 is a plot showing the measured fluorescent emission from the clump of beads shown in FIG. 8, and the measured background emission to the side of the clump of beads, as a function of time as the excitation light beam goes back and forth from being aimed at the clump of beads, and being aimed to the side of the clump of beads;

FIG. 10 schematically shows a system that can be used to perform the method of FIG. 6, similar to the system of FIG. 7, but using separate light beams to aim at the clump of beads, and to aim to the side of the clump of beads, according to an exemplary embodiment of the invention;

FIG. 11 schematically shows a system that can be used to perform the method of FIG. 6, similar to the system of FIGS. 7 and 10, but using a light beam split into two beams, one of them aimed at the clump of beads, and one of them aimed to the side of the clump of beads, according to an exemplary embodiment of the invention;

FIG. 12 schematically shows a system similar to the system of FIG. 5, for performing a plurality of bio-assays by measuring fluorescent emission from magnetic beads that are aggregated into a clump by a magnet beneath the bottom of a container, in each of a plurality of containers using different samples, for example in different wells of a well plate, according to an exemplary embodiment of the invention;

FIG. 13 schematically shows a system for performing a plurality of bio-assays, each in a different container by measuring fluorescent emission from magnetic beads that are aggregated into a clump by a magnet beneath the bottom of each container, using an optical fiber switch to sequentially excite and measure the fluorescent emission from the different containers, without a need for moving parts, according to an exemplary embodiment of the invention;

FIG. 14 schematically shows a system for performing a plurality of bio-assays, each in a different container by measuring fluorescent emission from magnetic beads that are aggregated into a clump by a magnet beneath the bottom of each container, using a microlens array to simultaneously illuminate the different containers from excitation light from a single light source, and to simultaneously measure fluorescent emission from the beads in the different containers, according to an exemplary embodiment of the invention;

FIG. 15 is a flowchart for a method of sequentially performing bio-assays for each of a plurality of wells in a well plate, by measuring fluorescent emission from magnetic beads that are aggregated into a clump by a magnet beneath that well, according to an exemplary embodiment of the invention;

FIG. 16 shows schematic views of two systems for performing the method of FIG. 15, according to an exemplary embodiment of the invention;

FIG. 17 is a flowchart for a method of performing a bio-assay in a microfluidics cartridge, by measuring fluorescent emission from magnetic beads that are aggregated into a clump by a magnet beneath the cartridge, according to an exemplary embodiment of the invention;

FIG. 18 shows a schematic top view of a microfluidics cartridge that can be used to perform the method of FIG. 17, according to an exemplary embodiment of the invention;

FIG. 19 schematically shows a system that can be used to perform the method of FIG. 17, using the cartridge of FIG. 18, according to an exemplary embodiment of the invention;

FIG. 20 shows the fluorescent emission signal as a function of the number of fluorescent molecules attached to each magnetic beads, and the fluorescent emission signal as a function of the concentration of human interlukin-8 in a sample, from tests done using a system similar to that of FIG. 16;

FIG. 21 shows the fluorescent emission signal for assays performed using a system similar to that of FIG. 16, for the SARS-Cov-2 virus, for subjects who were known to be positive for the virus and for subjects who were known to be negative for the virus;

FIG. 22 shows the strong correlation between the fluorescent emission measured using the method of FIG. 6 and a system similar to that of FIG. 16, according to an exemplary embodiment, and the fluorescent emission measured using a Magnetically Modulated Biosensing (MMB) system and method;

FIG. 23 shows a schematic 3-D perspective view of a system for performing an assay by measuring the fluorescent emission of magnetic beads aggregated at the bottom of a cuvette, and correcting the measurement for background emission, showing only elements of the system used for illuminating the beads with excitation light, according to an exemplary embodiment of the invention;

FIG. 24 shows a schematic side view of part of the system of FIG. 23, showing some of the optical elements used to measure the fluorescent emission;

FIGS. 25A and 25B respectively show plots of a fluorescent emission signal from magnetic bioassay beads during and after their aggregation as a function of time, for two different concentrations of target molecules in the sample, with the illumination of the beads by the excitation light periodically interrupted so that the photobleaching rate can be measured, illustrating the dependence of the photobleaching rate and the aggregation rate on the concentration of target molecules, according to an exemplary embodiment of the invention;

FIGS. 26A and 26B respectively show a fluorescent emission signal from magnetic bioassay beads during and after aggregation of the beads as a function of time, with the illumination of the beads by the excitation light periodically interrupted with two different illumination duty cycles, showing that a lower duty cycle results in less photobleaching and consequently a higher fluorescent emission level, according to an exemplary embodiment of the invention;

FIG. 27 shows a flowchart for a method of performing a bioassay of a sample by measuring a fluorescent emission signal from an aggregating clump of magnetic bioassay beads, before, during and after aggregation, while periodically interrupting illumination of the beads by an excitation light, using the emission signal as a function of time to determine a concentration of a target molecule in the sample, according to an exemplary embodiment of the invention;

FIGS. 28A and 28B respectively show a side view and a top view of magnetic bioassay beads trapped inside a microfluidic channel by a magnet adjacent to the channel, while a fluid flows past the beads, washing away molecules that would produce background fluorescent emission, according to an exemplary embodiment of the invention; and

FIG. 29 shows a flowchart for a method of performing a bioassay using magnetic bioassay beads trapped inside a microfluidic channel, with fluid flowing past the beads washing away molecules that would produce background fluorescent emission, according to an exemplary embodiment of the invention.

### DESCRIPTION OF SPECIFIC EMBODIMENTS OF THE INVENTION

The present invention, in some embodiments thereof, relates to bioassays and, more particularly, but not exclusively, to bioassays using fluorescent reporter molecules and magnetic beads.

An aspect of some embodiments of the invention concerns a bioassay method and system, in which the concentration and/or the quantity of a target molecule in a sample, and/or the presence or absence of the target molecule in the sample, is determined by measuring optical emission from reporter molecules, for example fluorescent reporter molecules, that are attached to magnetic beads in a solution in a container, and the magnetic beads are aggregated into a clump in 60 seconds or less by a magnetic field, with a field gradient, produced inside the container by one or more magnets. The aggregation of many magnetic beads into a small area potentially results in a greater sensitivity, a greater precision, and/or a simpler and less expensive optical apparatus, for detecting and measuring the emission, than in some prior art methods where emission is measured from one bead at a time, or if the beads were spread out over a larger area. Optionally the clump forms on an inner surface of the container, in contact with the solution, optionally on a bottom inner surface, and takes up only a small fraction of the area of the part of the inner surface that is covered by the solution, for example less than 10% of the inner surface or less than 3% of the inner surface, or less than 1% of the inner surface that is covered by the solution. Optionally, the clump covers an area of the inner surface that extends at least 0.05 mm in all directions along the inner surface that it is located on, or at least 0.1 mm, or at least 0.2 mm, or at least 0.5 mm. Optionally, the beads are densely packed in the clump, and the beads cover at least 60% of that area of the inner surface, or at least 75%, of that area. Optionally, the clump is more than one layer of beads thick, and optionally the beads cover between 80% and 100% of that area. Optionally the magnetic beads are aggregated into the clump in between 60 and 40 seconds, or between 40 and 30 seconds, or between 30 and 20 seconds, or between 20 and 15 seconds, or between 15 and 10 seconds, or between 5 and 10 seconds, or between 3 and 5 seconds, or less than 3 seconds. Optionally, the beads are aggregated into the clump in this time, from throughout the volume of solution in the container. Alternatively, the beads are aggregated into the clump in this time from only part of the volume of solution in the container, for example from more than 70% of the volume, or from between 50% and 70% of the volume, or between 30% and 50% of the volume, or between 20% and 30% of the volume of solution in the container, and magnetic beads in the rest of the volume of solution in the container take more time to aggregate into the clump, or never aggregate into the clump. Even if some of the beads take a longer time to aggregate into the clump, or never aggregate into the clump, the beads that do aggregate into the clump in the shorter time may produce a strong enough optical emission signal to perform the assay at a desired level of precision and/or sensitivity, and with a desired throughput rate. As used herein, the “aggregation time” of the beads means the aggregation time for the beads that aggregate into the clump from which the optical emission is measured. There may also be other beads present in the solution, that fail to aggregate in that time. Optionally, some of the beads aggregate into the clump, and some of the beads aggregrate into at least one other clump. Optionally, the optical emission is also measured from at least one of the at least one other clumps, and the measurement is optionally used for the assay; alternatively the optical emission is not measured from any other clump.

Optionally, the bioassay method comprises preparing the magnetic beads so that an average number of reporter molecules attached to each bead depends on, and is optionally proportional to, the concentration and/or the quantity of the target molecules in the sample. It should be understood that it is generally possible to calculate the concentration of target molecules from the quantity of target molecules, for example the weight or number of target molecules, and vice versa, if the quantity of the sample is known, for example the weight or volume of the sample. Finding the concentration of the target molecules is generally equivalent to find the quantity of the target molecules, and for conciseness only finding the concentration of target molecules will generally be referred to, herein. For example, the bioassay is a sandwich assay, or an energy transfer assay, for example using fluorescent reporter molecules. Then the measured optical emission from the beads can be used to determine the concentration of target molecules in the sample, and/or to determine the presence or absence of target molecules in the sample. Improving the sensitivity or precision of measuring the optical emission for the beads will improve the sensitivity and/or precision of the assay.

Optionally, the magnetic field inside the container remains unchanged, or changes relatively little, during the aggregation time of the beads into the clump, and/or during the time that the optical emission is measured. Optionally, the magnetic field changes by less than 10% in magnitude, and/or by less than 0.1 radians in direction, within the volume of the solution, or within the part of the volume of the solution from which the beads are aggregated, during the aggregation time before the optical emission is measured, and/or during the time that the optical emission is measured from the clump of beads. Additionally or alternatively, the gradient of the magnetic field, or the components of the gradient, which also affects the magnetic force on the beads, change by less than 10% in magnitude, and/or by less than 0.1 radian in direction, within this volume, during this time. Optionally the magnetic field is produced predominantly by one of more permanent magnets, or only by one or more permanent magnets, which remain fixed in position relative to the container when the beads are aggregating before the optical emission is measured and/or when the optical emission is being measured, and the magnetic field inside the container substantially does not change at all when the beads are being aggregated before the optical emission is measured and/or when the optical emission is being measured.

Using one or more permanent magnets, or using only permanent magnets, to produce the magnetic field, has the potential advantage that the one or more magnets can be more compact than an electromagnet that produces the same field, and they do not consume any power. Using smaller magnets may allow the container to be smaller, and may allow a large number of containers to be used close together to perform multiple assays of different samples, for example using the wells of a standard 96-well plate. Optionally, the permanent magnet material has a relatively high energy product, for example at least 100 kilojoules per cubic meter, or at least 200 kilojoules per cubic meter, or at least 300 kilojoules per cubic meter, for example a permanent magnet material comprising a rare earth element, such as a neodymium iron boron magnet, or a samarium cobalt magnet. Using a permanent magnet material with a high energy product has the potential advantage that a higher magnetic field and magnetic field gradient can be produced in the container, and/or that the magnet or magnets can be smaller, and/or can be shorter relative to their diameter, than for a lower energy permanent magnet material, such as Alnico. A potential advantage of having smaller or shorter magnets has been noted above. Having a higher magnetic field and a higher magnetic field gradient may result in a faster aggregation time for the magnetic beads. Alternatively, a lower energy product permanent magnet material is used.

Optionally, the one or more magnets that produce the magnetic field inside the volume of the solution are located outside the container, and are much closer to a small part of the inside surface area of the container where it is covered by the solution, than to most of the rest of the inner surface where it is covered by the solution. Having the magnets positioned closer to a small part of the inner surface may produce a magnetic field that has a higher magnitude and/or a higher gradient near that part of the inner surface, and that exerts a greater magnetic force on the beads, than at most other parts of the inner surface, and that aggregates the beads into a clump much smaller in area than the whole inner surface that is covered by the solution, adjacent to that part of the inner surface.

Optionally, the one or more magnets are located beneath the bottom of the container, optionally close to the container. Optionally, even if all of the one or more magnets are not located beneath the bottom of the container, a small area of the inner surface which is much closer to the one or more magnets than most of the inner surface, and/or a small area of the inner surface where the magnetic field exerts a much greater magnetic force on one of the beads than over most of the inner surface, is located on the bottom of the container. Having the magnets much closer to a small area on the bottom of the container, and/or having a greater magnetic force on the beads over the small area on the bottom of the container, may cause the beads to aggregate into a clump on the bottom of the container, adjacent to the small area. Optionally, the magnetic field causes the beads to aggregate into a clump on the bottom of the container, optionally into a clump that covers only a small fraction of the area of the bottom of the container or of the whole inner surface of the container that is covered by the solution, for example less than 20% or between 10% and 20% or between 5% and 10% or between 2% and 5% or between 1% and 2% or less than 1% of the area of the bottom of the container, or of the whole inner surface of the container that is covered by the solution.

Having the beads aggregate into a clump on the bottom of the container has the potential advantage that the solution need not fill the container beyond a relatively low depth, because the solution only has to be deep enough to be sure to cover the bottom of the container, and the clump of aggregated beads. If the one or more magnets are located instead closest to a small area on the side of the container, in order to aggregate the beads there, then the depth of solution must be great enough to come well above the level of the small area on the side, in order to aggregate the beads into a clump in that area. Aggregating the beads at the bottom of the container may allow a smaller sample to be used for the assay, than if the solution had to reach to a higher level above the bottom of the container. For example, the solution is less than 10 mm deep above the clump, or between 5 and 10 mm deep above the clump, or between 4 mm and 5 mm deep, or between 3 and 4 mm deep, or between 2 and 3 mm deep, or less than 2 mm deep above the clump. Optionally the depth of solution in the container is lower than the width of the solution in all horizontal directions. For example, the container is a circular well 6 mm in diameter, as in a standard 96-well plate, and the depth of solution is less than 6 mm, or less than 5 mm or 4 mm or 3 mm or 2 mm. Having a lower level of solution in the container also has the potential advantage that there may be a lower noise level, in measuring the optical emission if the optical emission comprises fluorescent emission, due for example to less Raman scattering of a fluorescent excitation beam from the solution, or less fluorescent excitation from fluorescent molecules dissolved in the solution. Having the one or more magnets located beneath the bottom of the container also has the potential advantage that the wells of a standard well-plate, for example a standard 96-well plate, may be used to perform multiple assays with different samples, and there will still be room to bring the one or more magnets close to the bottom of the well, even if there is not enough room between the wells in the well plate to bring a magnet close to the side of the well.

Optionally, the horizontal dimensions of the container are also relatively small, throughout the height of the container up to the top of the solution, or at least the horizontal dimensions of the container are relatively small at the bottom of the container, for example smaller than 10 mm in all horizontal directions, or at least in one horizontal direction, or smaller than 5 mm in all horizontal directions, or at least in one horizontal direction, or smaller than 3 mm in all horizontal directions, or at least in one horizontal direction, or smaller than 2 mm in all horizontal directions, or at least in one horizontal direction.

The inventors have found that aggregating the magnetic beads into a clump at the bottom of the container, and using a container of such small dimensions, results in a much faster aggregation of the magnetic beads, and consequently a much higher throughput for the assay, compared to other configurations used by the inventors for magnetically aggregating beads in an assay, such as the Magnetically Aggregated Biosensing (MAB) assay, in which the magnetic beads are aggregated by a magnet located on the side of the container, or the Magnetically Modulated Biosensing (MMB) assay, in which the magnetic beads are alternately aggregated by two magnets, generally electromagnets, located at opposite sides of the container. Furthermore, in the case of the MMB assay, where there are two magnets at opposite side of the container that alternately aggregate the beads, the assay generally works best if the distance between the magnets, and hence the width of the container in that direction, is not too great, for example a distance between the tips of the magnets of 1.2 mm, and a width of the inside of the container of 0.5 mm. In this case, the container will typically be much wider than that width in its other two dimensions, and the depth of the solution will typically be greater than 2 mm, or greater than 5 mm, or greater than 10 mm, if the total volume is as great as a typical volume of the sample that is used for the assay. And it is difficult to fill a container with the solution if it has walls that are only 0.5 mm or even 1 mm apart.

Optionally, one or more of the one or more magnets, or all of the magnets, are located outside the container, for example just outside the container, and are magnetized in a direction approximately normal to the inner surface of the container that the magnet is closest to, and have a magnetic dipole moment approximately in that direction. For example the one or more magnets are magnetized in a direction within 30 degrees, or between 20 and 30 degrees, or between 10 and 20 degrees, or less than 10 degrees, from a direction normal to the inner surface at a point that the magnet is closest to. For example, if the magnet is closest to a bottom surface of the container, where the inner surface is approximately horizontal, then the magnet is magnetized, and has a dipole moment, in a direction within 30 degrees, or between 20 and 30 degrees, or between 10 and 20 degrees, or less than 10 degrees, from vertical. Having a single magnet, or a plurality of magnets clustered close together, magnetized in a direction nearly normal to the inner surface, may produce a magnetic field and a magnetic field gradient that are oriented nearly normal to the surface near the point which is closest to the magnet or magnets, and may produce a magnetic force that pulls the beads to a location on the inner surface that is closest to the magnet or magnets, for example a location at the bottom of the container, causing the clump to aggregate around that location.

Optionally the one or more magnets comprise a magnet that has a tip, and optionally the tip has a sharp point, for example the tip is cone-shaped, or parabolic, with a radius of curvature, at the point of the cone, less or even much less than the radius of the magnet, for example less than 3 mm, or less than 2 mm, or less than 1 mm, or less than 0.5 mm, or less than 0.3 mm. Optionally the magnet is adjacent to the outside surface of the container, and optionally the tip of the magnet, optionally the sharp point of the tip, is the part of the magnet that is closest to the outside surface of the container. For example, the magnet, optionally the tip of the magnet, touches the outside surface of the container, or is within a distance from the outside surface of the container, or from the inside surface of the container, that is much smaller than the diameter of the magnet, or smaller than 5 mm, or smaller than 3 mm, or smaller than 2 mm, or smaller than 1 mm, or smaller than 0.5 mm, or smaller than 0.3 mm, or smaller than 0.2 mm, from the outer surface of the container, or from the inner surface of the container. Optionally, the tip is not made of permanent magnet material, but is made of a soft magnetic material, meaning a material with relatively low coercivity, and the tip is added to the end of the permanent magnet. Optionally, the tip is made of a material with relatively high saturation field Bₛₐₜ, such as iron, or Hiperco 50, or a similar alloy. Alternatively, the tip is made of permanent magnet material, for example the same material as the rest of the magnet. Having a magnet magnetized approximately in a direction normal to the surface of the container, with a sharp tip of a material with high Bₛₐₜ adjacent to the container, has the potential advantage that the magnetic field B and its gradient VB inside the container will both be relatively high, and will both be oriented approximately in a direction toward the magnet and normal to the surface of the container, at a location inside the container closest to the tip of the magnet. Then the magnet will exert a relatively large magnetic force on the beads just inside the container adjacent to the magnet, and the magnetic force on the beads will also be relatively large further away from the magnet in the container, which may cause the beads to aggregate more quickly than if the magnetic force were further away from the container, or further from being in a direction normal to the surface of the container. In the tests done by the inventors and described below in the “Examples” section, for example, there is a single magnet with a sharp cone-shaped tip, located beneath the bottom of the container, and the magnetic force draws the beads downward into a clump at the bottom of the container just above the tip of the magnet.

Optionally, the solution comprises a buffer solution. Additionally or alternatively, the solution comprises a liquid portion of the sample, for example a human or animal sample such as whole blood, or blood plasma, or urine, or saliva, or a liquid sample from a plant or processed food, or an environmental sample, such as a sample from a water reservoir, lake, river, or sea.

Optionally, the method and system are designed for use with a particular type of magnetic beads, for example with any particular type of magnetic beads that is known for use in bioassays. For example, M-280 superparamagnetic beads (ThermoFisher Sci. Waltham, MA, USA) were used for some of the tests described below in the Examples section, and in some of the tests Magplex beads (from Bio-Rad) were used. The time needed to aggregate the beads into a clump, which is often the bottleneck for throughput of the assay, and the sensitivity of the assay, may depend on the type and quantity of beads, the dimensions of the container and solution, and the size, shape, strength, orientation and position of the magnet relative to the container. Optionally, the depth of the solution in the container is small enough, and/or the horizontal dimensions of the container are small enough, so that the magnetic force on a bead, or the vertical component of the magnetic force, is greater than the net gravitational force on a bead, defined as the gravitational force minus the buoyancy force, for the entire volume of the container up to the top of the solution, or for most of the volume of the container up to the top of the solution, or for all of the area of the bottom of the container, or for most of the area of the bottom of the container. Optionally, the magnetic force on a bead, or the vertical component of the magnetic force, is at least as great, or at least twice as great, or at least 5 times as great, or at least 10 times as great, or at least 20 times as great, or at least 50 times as great, as the net gravitational force on a bead, over all of the volume, or most of the volume up to the top of the solution, or over all or most of the area of the bottom of the container.

Optionally the dot product of the magnetic field B produced by the magnet, and the magnetic field gradient VB produced by the magnet, which may determine the magnetic force on the beads, is greater than 1 T²/m everywhere in the container up to the top of the solution, or greater than 0.5 T²/m, or greater than 0.2 T²/m, or greater than 0.1 T²/m, or greater than 0.05 T²/m, everywhere in the container up to the top of the solution. Optionally, the time that a magnetic bead would require to travel from anywhere in the container to a location at the bottom of the container that has the most intense magnetic field, if the bead travels at a velocity for which its viscous drag in water would balance the magnetic force on the bead at each location that it passes, is less than 50 seconds, or less than 20 seconds, or less than 10 seconds, or less than 5 seconds, or less than 3 seconds.

Without being limited by any theory as to why the aggregation time is observed to be much shorter in configurations where the magnet is just below the bottom of the container than in configurations where the magnet is adjacent to the side of the container, the inventors believe that the aggregation time may depend on the time needed to attract the most distant beads in the container to the location closest to the tip of the magnet, where the clump of beads eventually forms. The aggregation time may be shorter if the magnetic force, attracting the beads toward the tip of the magnet, is greater than the gravitational force throughout the container, or throughout most of the container, especially if the magnetic force is vertical and adds to the gravitational force, or if the magnetic force on the beads, attracting them horizontally to the tip of the magnet, is relatively great throughout the bottom of the container, or throughout most of the bottom of the container, once beads fall to the bottom of the container under the influence of the net gravitational force. It should be noted that, for a configuration where the magnet has a sharp tip just below the bottom of the container, the magnetic force rapidly increases as the beads get closer to the tip of the magnet, and this increase in magnetic force may be even greater when the density of magnetic beads near the magnet becomes great enough to make the average permeability in that region significantly greater than the permeability of free space. In these circumstances, the aggregation time may be dominated by the time needed for the beads to travel halfway to the tip of the magnet from a part of the container that is furthest from the tip of the magnet, with the beads travelling across the rest of the distance much more quickly. For a given total magnetic, gravitational and buoyant force on the bead, it is believed that the bead may quickly reach a terminal velocity such that its viscous drag force at that velocity in the solution balances the total magnetic, gravitational and buoyant force, so the velocity of a bead at any given time will be proportional to the total magnetic, gravitational and buoyant force on it. So the aggregation time may be reduced by having a relatively high value of B and high gradient of B adjacent to the tip of the magnet, both close to vertical, and having a container with a relatively small depth of solution, and relatively small horizontal dimensions, and a bottom of the container of relatively small area.

Optionally, the aggregation time for the magnetic beads is less than 30 seconds, or less than 20 seconds, or less than 10 seconds, or less than 5 seconds, or less than 3 seconds. These numbers are comparable to the observed aggregation times, and are comparable to the time needed for a magnetic bead to reach the bottom of the container, from a few millimeters above the tip of the magnet, assuming that at any given time the bead is moving at a velocity where the magnetic force on the bead is balanced by the viscous drag of the solution, which is assumed to have the viscosity of water. An estimate of this time is given below under the heading “Estimate of magnetic force and aggregation time,” and suggests the importance of having a container with a depth of solution that not too great, for example less than 3 mm, and a lateral extent from the tip of the magnet that is not too great, for example less than 3 mm in any direction, in order to achieve a rapid aggregation time.

Optionally, the optical emission of the magnetic beads is excited by an excitation light beam that illuminates the magnetic beads, for example the optical emission is fluorescent emission or phosphorescent emission. Alternatively, the optical emission is chemiluminescent emission, excited by exposure to a chemical, or electro-chemical luminescence, excited by an electric current. In the tests done by the inventors, and in the examples described herein, fluorescent emission is used, but it should be understood that, when fluorescent emission is mentioned herein, other types of optical emission may be used instead of, or in addition to, fluorescent emission.

Optionally, in measuring the fluorescent emission of the magnetic beads, a correction is made for background emission from the solution. If the fluorescent emission of the magnetic beads in the aggregated clump is excited by a beam of excitation light that illuminates the beads from above, passing through a first volume of the solution, and if the fluorescent emission of the beads is measured by viewing the beads from above, through the first volume of the solution, then any background emission coming from the first volume of the solution will add to the measured emission from the magnetic beads, especially any background emission that has the same or a similar range and distribution of wavelengths as the fluorescent emission, and cannot be easily excluded by filtering. Such background emission, at the same or similar wavelengths as the fluorescent emission from the beads, could come, for example, from Raman scattering of the excitation light by water molecules in the solution, and/or from any stray fluorescent molecules that are dissolved in the solution, particularly fluorescent molecules of the same type that are producing the fluorescent emission from the beads. To correct for this background emission, and to obtain a more accurate measurement of the true fluorescent emission from the beads, optionally the excitation light beam that is illuminating the beads is alternately aimed to the side of the clump of beads, through a second volume of the solution, and the background emission from the second volume is measured. The background emission from the first volume is estimated from the measured background emission from the second volume, for example the two are assumed to be nearly the same, and the estimated background emission from the first volume is subtracted from the measured fluorescent emission from the beads, to obtain a corrected value for the fluorescent emission from the beads.

Even if the light beam has the same cross-section, and goes through the same depth of solution when illuminating the clump of beads and when passing to the side of the clump of beads, so that the first volume is the same size and shape as the second volume, the background emission from the first volume may be less than the background emission from the second volume, due to more light being absorbed, and scattered to the sides, when the light beam is illuminating the clump of beads, and more light being reflected back up into the second volume, when the light beam is passing to the side of the clump and hitting the smooth bottom of the container. Alternatively, the background emission from the first volume may be greater than from the second volume, due to more light being reflected from the beads than from the bottom of the container, especially if the bottom of the container is transparent and most of the light beam passes through it when it goes to the side of the clump of beads. Optionally these effects, which may be reduced by using a shallower depth of solution, are taken into account when estimating the background signal from the first volume, using the measured background signal from the second volume. The inventors have found that these effects are relatively small for the tests described below in the “Examples” section.

It should be noted that the configuration described here, for performing an Optically Modulated Biosensing (OMB) assay with the excitation light beam coming from above and illuminating the clump at the bottom of the container, may allow a more accurate correction for background emission than the usual configuration for a Magnetically Modulated Biosensing (MMB) assay, and hence may allow a more sensitive assay. In an MMB assay, as described for example in the references cited above, the clump of beads is typically close to the front of the container on the side from which the excitation light beam enters the container, so the excitation light goes through relatively little solution, when it is illuminating the clump of beads. When the clump of beads has moved to the side of the excitation light beam, however, the excitation light beam travels a much greater distance into the container, which is typically much deeper in the direction of the excitation light beam, than in the direction of the magnetic force on the beads. Hence, the background signal seen when the excitation light beam is going to the side of the clump is much greater than the background signal when the excitation light beam is illuminating the clump, and it may be difficult to accurately correct for the background signal when the light beam is illuminating the clump. In the present configuration, the excitation light beam goes through nearly the same depth of solution when it is illuminating the clump, and when it is passing to the side of the clump, so the background signal in the two cases will be nearly the same, and it may be easier to accurately correct for the background signal when the excitation light is illuminating the clump, potentially improving the sensitivity of the assay compared to an MMB assay. The OMB assay described here also potentially has greater sensitivity than the Magnetically Aggregated Biosensor (MAB) assay described above in the Background section, because the MAB assay does not correct the fluorescent emission signal for the background signal at all.

Improved optical sensitivity of the assay can reduce processing time in the case of a PCR test, because fewer PCR cycles may be needed to amplify the concentration of the DNA target molecule in the sample to a level where it can be detected and/or measured. And for any kind of assay using fluorescent beads, having greater optical sensitivity may mean that fewer washing and separation steps are needed to separate the beads from water that may have dissolved fluorescent reporter molecules that produce a background signal, which also can reduce processing time.

Alternatively, instead of aiming the same beam of excitation light alternately through the first volume to the beads, and through the second volume to the side of the beads, a second light beam, optionally with substantially the same wavelength distribution, cross-section, and intensity as the first beam of excitation light that illuminates the beads, is used to illuminate the second volume to the side of the beads. Optionally, the two beams are produced by splitting a beam from a same light source. As used herein, “substantially similar” means that the wavelength distribution, beam cross-section, and intensity of the two beams are close enough, and the depth of solution that they go through are close enough, that the expected emission from the first volume will differ from the emission from the second volume, due to any one of these differences, by less than 30%. Optionally, the expected emission will differ by less than 20%, or by less than 10%, or by less than 5%. Using two beams with substantially the same wavelength distribution, cross-section, and brightness, and having both beams go through nearly the same depth of solution, has the potential advantage that the expected background emission from the first volume will be nearly the same as the measured background emission from the second volume. Optionally, known differences between the two beams are taken into account, in calculating an expected difference in the background emission from the first and second volumes, and this expected difference is taken into account in estimating the background emission of the first volume, from the measured background emission of the second volume.

Optionally, if two different light beams are used, then the first beam illuminates the beads at the same time as the second light beam illuminates the second volume to the side of the beads, and emission from the beads and the first volume is distinguished from the emission from the second volume, by a camera that makes an image of the bottom of the container, including both the clump of beads and the area that the second beam is aimed that. Alternatively, the two beams illuminate the solution alternately at different times, for example by being turned on and off alternately, or by being blocked by shutters, such as mechanical shutters or Kerr cells, that are opened and closed alternately. In this case, or if a single light beam goes back and forth between illuminating the beads through the first volume, and illuminating the second volume, then the emission from the beads and the first volume is optionally distinguished from the emission from the second volume by their timing, instead of or in addition to making an image of the bottom of the container to distinguish the two emission measurements.

Optionally, the excitation light beam illuminates the clump of beads substantially vertically from above, defined herein as within 30 degrees of vertical. Optionally, the excitation light beam illuminates the clump of beads from within 20 degrees, or within 10 degrees, or within 5 degrees of vertical. Optionally, the solution is less than 10 mm, or less than 5 mm, or less than 3 mm, or less than 2 mm deep, above the clump of beads.

Any kind of light sensing device known in the art may be used for measuring the fluorescent emission of the clump of beads, and for measuring the background emission from the second volume. Suitable optical elements, such as lenses, are optionally used to bring the fluorescent emission light and background light from the container to the light sensing device. Optionally, both measurements are made with a same light sensing the device, which has the potential advantage that the correction for background emission from the first volume may be more accurate than if two different light sensing devices are used. Alternatively, different light sensing devices are used. The light sensing device or devices comprise, for example, a single photo cell, for example a photo cell that receives and measures an integrated light emission coming from the container, or coming from a field of view of the container that includes the clump of beads, and optionally the first and second volumes. Alternatively, the light sensing device or devices comprises an array of photo cells, or a camera, such as a CMOS or CCD camera that comprises an array of photo cells, that optionally produces an image of a field of view of the container, including the clump of beads, and the first and second volumes. Using a light sensing device that produces an image has the potential advantage that it can distinguish fluorescent emission from the beads, including background emission from the first volume, from background emission from the second volume, even if the beads and the second volume are illuminated at the same time. Using a single light sensor that integrates light received from the container has the potential advantage that it may have a lower noise level than the pixels of a camera or photo array, and it may be less expensive and more robust.

In some embodiments of the invention, the assay is multiplexed, used to simultaneously measure the concentration of two or more different target molecules in the same sample. This is done, for example, by using two or more different probe molecules, that each bind specifically to a different target molecule, and that each have a different fluorescent reporter molecule attached, that produces fluorescent emission in different wavelength ranges. In this way, by measuring the fluorescent emission in the two wavelength ranges at the same time, assays for the two target molecules are performed at the same time, increasing throughput.

Optionally, the target molecule that the assay detects and/or measures is a DNA molecule or an RNA molecule, of a specified sequence, or containing a specified sequence, of nucleotides. If the target molecule is a DNA molecule, then the probe molecule optionally comprises a DNA molecule with nucleotide sequence complementary to the specified sequence of the target molecule. If the target molecule is an RNA molecule, then optionally corresponding DNA molecules are produced from any RNA molecules in the sample using reverse transcriptase, and the probe molecule optionally comprises a DNA molecule with nucleotide sequence complementary to the DNA sequence of the DNA molecule that would be produced from the target RNA molecule by reverse transcriptase. Optionally, the target molecule is an antibody and the probe molecule is a protein that the antibody specifically binds to, or the target molecule is a protein and the probe molecule is an antibody that binds specifically to that protein.

Optionally, the assay is performed using a well plate with a plurality of wells, for example using a standard well plate with 96 wells, or using a plurality of different containers. It should be understood that, whenever the use of a plurality of wells in a well plate is described herein, a plurality of different containers, not necessarily part of a well plate, can be used instead.

A potential advantage of the assay method described here, over the Magnetically Modulated Biosensing (MMB) method, is that it is possible to perform the assay in a conventional well plate, because the excitation light comes from above, and the fluorescent emission is measured from above, and the well plate can be moved horizontally to measure the fluorescent emission from different wells sequentially. With an MMB assay, relatively bulky electromagnets are placed just outside opposite side walls of a container, oriented with their long axes facing the container, and it would be difficult to find room for such magnets between the wells of a standard well plate where the wells are arranged in a two-dimensional array. Furthermore, the wells of a standard well plate are too wide for an MMB assay to work well, because the magnets would have to be too far apart.

Optionally, a plurality of assays are performed in different wells of the well plate, using all of the wells in the well plate, or only using some of the wells in the well plate. Optionally, the beads are prepared using different samples in different wells of the well plate, optionally simultaneously, and the fluorescent emission from the beads used for each assay is measured in the same well where the beads for that assay were prepared. Alternatively, the beads for each assay are prepared elsewhere, and are then transferred to different wells of the well plate to measure their fluorescent emission. Optionally, each well in the well plate has its own magnet located below the well, adjacent to the bottom of the well. Alternatively, one magnet is used for a plurality of different wells, and before measuring the fluorescent emission from each of these wells, the magnet is moved, or the well plate is moved, or both, so that the magnet is located beneath and adjacent to the bottom of each well when the fluorescent emission from the beads in that well is to be measured. Even in that case, there is still optionally more than one magnet for each well plate, and the fluorescent emission is measured simultaneously for more than one well, each with a different magnet positioned beneath it.

Alternatively, there is only a single magnet for the entire well plate, and the fluorescent emission is measured at different times for different wells. Using different magnets for different wells has the potential advantage that the magnetic beads can be aggregated into a clump in different wells simultaneously. Since the time needed to aggregate the beads may be the bottleneck that determines the throughput for performing assays, such an arrangement may increase the throughput. Using one magnet for different wells, one after the other, has the potential advantages that it may be less expensive, and may take up less room, than having a different magnet for each of the wells, and there will not be any tendency for magnets beneath neighboring wells to demagnetize each other, or for the magnetic field produced by one magnet to distort the magnetic field in a neighboring well.

Optionally, if the fluorescent emission of the beads is measured for a plurality of different assays in different wells of a well plate, a separate illumination sub-system is used for illuminating the beads in each of these wells with excitation light, with each sub-system having its own light source and its own optics, and/or each of these wells has its own detection system, with a separate light detecting device and associated optics for each of these wells, and optionally the fluorescent emission from the beads in the different wells is measured simultaneously. Alternatively, a single light source is used for a plurality of wells, but it is used to illuminate different wells simultaneously, for example using a plurality of lenslets, with a different lenslet for each well that is being simultaneously illuminated, and/or a single light detecting device is used measuring the fluorescent emission from the beads in each well simultaneously, for example with different lenslet used to focus the fluorescent emission from each of these wells on a different part of an array of light sensors, and optionally the fluorescent emission from the different wells is still measured simultaneously. Measuring the fluorescent emission from each of a plurality of wells simultaneously has the potential advantage that it may increase the throughput of the assays. Using a single light source, and/or a single light detector, to do this, has the potential advantage that it may be less expensive, and/or take up less room, than having separate light sources and/or separate light detecting devices for different wells.

Alternatively, a single light source is used to provide excitation light for each of these wells, and/or a single light detecting system is used for measuring the fluorescent emission from each of these wells, and the fluorescent emission is measured from the different wells sequentially. Optionally, there is a single illumination sub-system, and/or there is a single detection sub-system, and the well plate is moved, relative to the illumination sub-system and/or the detection sub-system, so that the illumination sub-system is positioned to illuminate each well, and the detection sub-system is positioned to measure the fluorescent from each well, before the fluorescent emission is measured from that well.

Alternatively, instead of moving the illumination sub-system relative to the well plate, and/or moving the detection sub-system relative to the well plate, there is an optical fiber, or an optical cable comprising an array of optical fibers, positioned to illuminate each of these wells, and/or positioned to receive fluorescent emission from each of these wells, and an optical fiber switch, optionally with no moving parts, sequentially connects the optical fiber or cable associated with each well, to the light source and/or to the light detector. This configuration has the potential advantage, over moving the well plate relative to the illumination sub-system and/or the light detection sub-system, that it need not have moving parts. Using optical cables each comprising an array of optical fibers has the potential advantage, over using single optical fibers, that with an optical cable that light detection sub-system can potentially record an image of each well, rather than only recording a measurement of total emission light received from each well.

It should be understood that some wells in a well plate can have their fluorescent emission measured simultaneously, while other wells have their fluorescent emission measured sequentially, by using magnets, illumination sub-systems, and/or light detection sub-systems that are used simultaneously for some wells in the well plate but not for all wells in the well plate, using any of the configurations described above.

Optionally, the assay, including measuring the fluorescent emission from the beads, is performed in a microfluidic cartridge, for example a disposable microfluidic cartridge. Using a microfluidic cartridge has the potential advantage that, once a sample is entered into the cartridge, the entire assay can be performed automatically or semi-automatically, potentially even in a setting, such as an underdeveloped country, where there is a lack of trained people and/or a lack of expensive equipment for performing assays. Another potential advantage of using a microfluidic cartridge for measuring the fluorescent emission is that the detection chambers in microfluidic cartridges typically have very small dimensions, which may speed up the aggregation time of the magnetic beads, potentially increasing the throughput for performing assays, which may be dominated by the aggregation time of the magnetic beads. Still another potential advantage of using a microfluidic cartridge, is that the sample and reagents interact in a closed volume, so the sample cannot accidentally contaminate other containers or other equipment used for other assays with different samples, which could possibly happen with an assay done in a well plate with wells that are open on top.

Optionally, the microfluidic cartridge is used in conjunction with a machine that performs the assay automatically or semi-automatically, once the sample is entered into an input port of the cartridge. For example, the machine optionally includes actuators, controlled by a controller such as a programmed computer or other circuitry, that at different times create increases or decreases in pressure in different chambers of the cartridge from outside the cartridge, allowing the sample to be moved to different chambers where it undergoes one or more of filtering, mixing a specified volume of the sample with a specified volume of buffer solution, incubating for a specified time with specified amounts of probe molecules, other reagents, and magnetic beads that are stably stored in the cartridge before it is used, exposing the magnetic beads to a magnetic field from a magnet that aggregates the beads into a clump, illuminating the clump with excitation light, measuring fluorescent emission from the clump, and moving one or more of the sample, the solution, and the beads into a waste chamber after the fluorescent emission is measured.

Alternatively, the microfluidics cartridge is used for measuring the fluorescent emission of the beads, but the earlier steps of the assay, preparing the beads so that their fluorescent emission depends on the concentration of the target molecule in the sample, is done outside the microfluidics cartridge, and the prepared beads are then entered into the cartridge. This has the potential advantage that it is not necessary to store buffer solution, reagents, and magnetic beads for a long period of time in the cartridge before using it, which may result in the cartridge having a short shelf life if the buffer solution, reagents, and magnetic beads have a short shelf-life.

An aspect of some embodiments of the invention concerns a bioassay method and system, in which the concentration and/or the quantity of a target molecule in a sample, and/or the presence or absence of the target molecule in the sample, is determined by measuring optical emission from reporter molecules, for example fluorescent reporter molecules, excited by an excitation light, that are attached to magnetic beads in a solution in a container, and a photobleaching rate of the beads by the excitation light is measured. Optionally, the measured photobleaching rate is taken into account in evaluating the results of the assay. For example, the measured optical emission is corrected for the effect of photobleaching. Alternatively or additionally, the concentration of target molecules in the sample is determined from a combination of the measured optical emission from the beads, and the measured photobleaching rate, making use of a correlation between the photobleaching rate of the beads and the concentration of target molecules in the sample, which the inventors have found. Optionally, the magnetic beads are aggregated into a clump on an inner surface of the container by a magnet located just outside the container, for example aggregated into a clump on a bottom surface by a magnet located just below the container, optionally aggregated in less than 60 seconds, and the optical emission is measured from a large number of the beads in the clump. Optionally, the excitation light alternately illuminates and does not illuminate the beads, for example by periodically turning a source of the excitation light on and off, or by periodically blocking and unblocking the excitation light from illuminating the beads, or by periodically adjusting a beam of the excitation light to illuminate the beads and to be directed off to the side of the beads. Optionally, the excitation light photobleaches the beads, and the photobleaching rate is measured, when the excitation light is illuminating the beads, and the beads recover from the photobleaching, at least partially, when the excitation light is not illuminating the beads. Allowing the beads to recover between intervals of photobleaching the beads may allow a more accurate measurement to be made of the photobleaching rate. Alternatively, the beads are illuminated continuously by the excitation light, for example during an extended time period when the beads are already fully aggregated into a clump that is not changing in size or configuration, for example during the entire time that the optical emission is measured for that assay, and the photobleaching rate is determined from a rate of decrease of the optical emission from the beads during the time period.

An aspect of some embodiments of the invention concerns a bioassay method and system, in which the concentration and/or the quantity of a target molecule in a sample, and/or the presence or absence of the target molecule in the sample, is determined by measuring optical emission from reporter molecules, for example fluorescent reporter molecules, excited by an excitation light, that are attached to magnetic beads in a solution in a container, and the excitation light alternately illuminates and does not illuminate the beads, for example as described above, in a repeating cycle. The excitation light photobleaches the beads while it is illuminating the beads, for example reducing the optical emission by at least 1% or at least 2% or at least 5% or at least 10%, and the beads substantially recover from the photobleaching when the excitation light is not illuminating the beads, for example recovering at least 80% or at least 90% or at least 95% of the emission coefficient (defined as the ratio of optical emission power to excitation light power) they had before photobleaching. Optionally, the net decrease in emission coefficient after each cycle of illuminating the beads and allowing them to recover is less than 5%, or less than 2%, or less than 1%, or less than 0.5%, or less than 0.2%, or less than 0.1%. Optionally, the total decrease in emission coefficient during all the cycles over which the emission from the beads is measured, is less than 50%, or less than 30%, or less than 20%, or less than 10%. The degree of photobleaching and the degree of recovery from photobleaching optionally depends on the intensity of the excitation light illuminating the beads, and on the cycle time, and on a duty cycle of the illumination time in each cycle, with greater recovery if the duty cycle of illumination is smaller. For example, the duty cycle of illumination is less than 50%, or less than 30%, or less than 20%, or less than 10%. As a result, the photobleaching may have less effect on the optical emission of the beads than if the beads were illuminated continuously for an extended time, and the measured optical emission of the beads may lead to a more accurate determination of the concentration of target molecules in the sample. Optionally, the magnetic beads are aggregated into a clump on an inner surface of the container by a magnet located just outside the container, for example aggregated into a clump on a bottom surface by a magnet located just below the container, optionally in less than 60 seconds, and the optical emission is measured from a large number of the beads in the clump.

An aspect of some embodiments of the invention concerns a bioassay method and system, in which the concentration and/or the quantity of a target molecule in a sample, and/or the presence or absence of the target molecule in the sample, is determined by measuring optical emission from reporter molecules, for example fluorescent reporter molecules, excited by an excitation light, that are attached to magnetic beads in a solution in a container. The beads are aggregated into a clump at a location on an inner surface of the container by one or more magnets located just outside the container, and light in the wavelength range of the optical emission, coming from the location, is measured as a function of time starting before the beads begin to aggregate, and continuing during the aggregation of the beads. Optionally, the optical emission measured as a function of time provides a more accurate determination of the concentration of target molecules in the sample, than the optical emission measured only after the beads are fully aggregated. For example, as found by the inventors, a rate of increase in the optical emission of the beads, between a time when the optical emission is 80% of its peak level, and a time when the optical emission is at its peak level, may be correlated with the concentration of target molecules at high concentrations of target molecules. Alternatively, a rate of increase in optical emission over other ranges of optical emission is used, for example between 0% and 30% of the peak level, or between 30% and 50% of the peak level, or between 50% and 70%, or between 70% and 80%, or between 80% and 90%, or between 90% and 100%, or any combination of these. At these high levels of concentration of target molecules, the optical emission level when the beads are fully aggregated may be relatively insensitive to the concentration of target molecules, or may even start to go down with increased concentration of target molecules, and making use of the additional information of the rate of increase of the optical emission may provide a more accurate measure of the concentration of target molecules. Optionally, the beads are aggregated onto a bottom surface of the container, by a magnet located just below the container. Optionally, the aggregation takes less than 60 seconds. Optionally the excitation light alternately illuminates and does not illuminate the beads, for example as described above, in a repeating cycle, with the excitation light photobleaching the beads when the beads are illuminated, and the beads at least partially recovering from the photobleaching when they are not illuminated. Optionally, the rate of photobleaching of the beads is measured, and the measurement of photobleaching is used to provide information on the concentration of target molecules in the sample, as described above.

An aspect of some embodiments of the invention concerns a bioassay method and system, in which the concentration and/or the quantity of a target molecule in a sample, and/or the presence or absence of the target molecule in the sample, is determined by measuring optical emission from reporter molecules, for example fluorescent reporter molecules, that are attached to magnetic beads in a fluid in a container. The container comprises a microfluidic channel, and one or more of the beads are trapped against a surface of the channel, for example at the bottom of the channel, by one or more magnets located just outside the channel, for example just below the channel, as the beads and the fluid flow along the channel. The fluid continues to flow past the trapped beads, washing away loose molecules that produce optical emission. This method has the potential advantage that a background contribution to the optical emission, due to loose molecules in the fluid surrounding the beads, may be greatly reduced, resulting in an accurate measurement of the optical emission from the beads, even without measuring and correcting for the background level of optical emission. Optionally a plurality of the beads, for example at least beads, or at least 100 beads, or at least 1000 beads, are trapped together as a densely packed clump on the surface of the channel, providing a larger optical emission signal than a single trapped bead would provide. Optionally there are two magnets, a first magnet located beneath the channel at a first location, and a second magnet located beneath the channel at a second location downstream from the first location, and at least some beads that fail to be trapped as they flow past the first magnet at the first location are trapped by the second magnet at the second location, providing a larger optical emission signal than if there were only a single magnet trapping beads.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

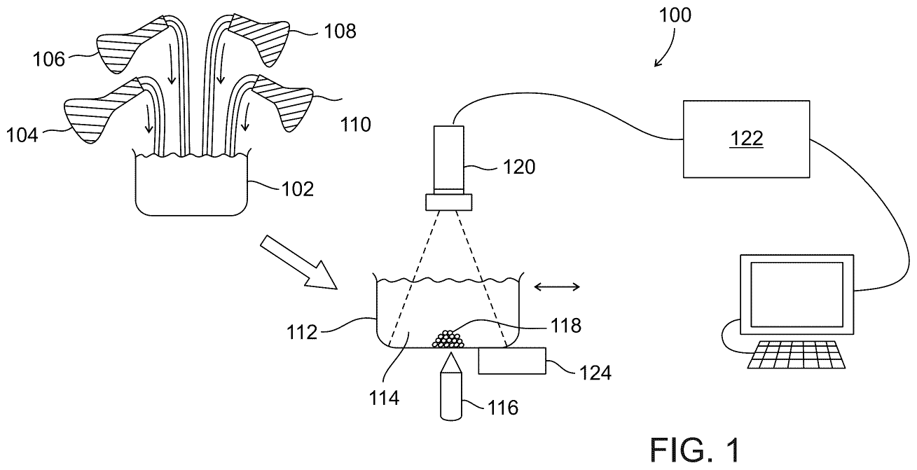

Referring now to the drawings, FIG. 1 schematically illustrates a system 100 for performing an assay, such as a bioassay, using optical emission from magnetic beads that are attracted to and aggregated by a magnet at or just below the bottom of a container where the optical emission from the beads is measured, according to an exemplary embodiment of the invention. In a container 102, a sample 104, such as a biological sample, is exposed to probe molecules 106, with attached light emitting reporter molecules. The probe molecules bind specifically to target molecules in the sample, such as specific DNA or RNA sequences or specific proteins or antibodies. In the case of a target molecule that is an RNA molecule, the RNA molecule is optionally used to produce a corresponding DNA molecule using reverse transcriptase, and the DNA molecule is used instead as the target molecule, with a complementary strand of DNA as the probe molecule. The probe molecules are also exposed to magnetic beads 108, and bind to the surfaces of the beads, either before or after the probe molecules are exposed to the target molecules, in container 102 or in a different container. Using assay reagents 110, in the same or a different container, to process the beads, the beads end up with reporter molecules bound indirectly to their surfaces that, when excited, emit light of an intensity that depends on how many of the probe molecules were bound to target molecules, and hence will depend on the concentration of target molecules in the sample.

Typically, the reporter molecule is a fluorescent reporter molecule, which emits fluorescent light in response to exposure to excitation light. The emitted light will often be referred to herein as fluorescent emission, the reporter molecule will often be referred to as a fluorescent reporter molecule, and the excitation light will often be referred to as fluorescent excitation light, because these are the most common cases. However, it should be understood that, whenever there is a reference herein to fluorescence, a different light emitting mechanism, excited by light, may be used, for example phosphorescence. Also, in cases where excitation by light is not a necessary part of a method or system that is described herein, light emission that is excited by a different mechanism, for example chemiluminescence or electro-chemiluminescence, may be used instead of fluorescence, even if this is not stated explicitly.

Optionally, the fluorescent probe molecules are prepared according to an energy transfer assay, for example a FRET assay. An energy transfer assay uses a labeled probe molecule that binds specifically to a target molecule that may be present in the sample. The label comprises a fluorescent reporter molecule and one or more dark quencher molecules in close proximity. Fluorescent emission from the fluorescent reporter molecule is reduced, typically by a large factor, by the presence of the dark quencher molecules. When the probe molecule binds to the target sequence, the fluorescent reporter molecule is disconnected from the dark quencher and more fluorescent emission is produced. The greater the concentration of target molecules in the sample, the more fluorescent reporter molecules will be disconnected from the dark quencher molecules, and more fluorescent light will be emitted for a given intensity of fluorescent excitation light illuminating the fluorescent reporter molecules. By measuring the fluorescent emission, the concentration of target molecules in the sample can be determined.

For the magnetic bead assays described here, the fluorescent reporter molecules are bound, for example indirectly bound, to the surfaces of magnetic beads, either before or after the labeled probe molecules are exposed to the sample and bind to any target molecules in the sample. In some embodiments of the invention, the fluorescent reporter molecules are bound to the surface of the magnetic beads by a biotin molecule, which is bound to the fluorescent reporter molecule, and an avidin molecule, which is bound to the surface of the magnetic bead. As used herein, this is considered indirect binding of the fluorescent reporter molecule to the surface of the bead, even though the biotin molecule may already be bound to the labeled probe molecule, and the bead may already be coated with avidin, before the assay begins.

The dark quencher molecule employs an energy transfer mechanism. Exemplary energy transfer mechanisms include, but are not limited to, fluorescent energy transfer (FET), also referred to as direct energy transfer, and fluorescence resonance energy transfer (FRET). The exact energy transfer mechanisms in these two cases are different. Further details on these two energy transfer mechanisms are provided, for example, in columns 14 and 15 of U.S. Pat. No. 8,465,989 to Adi Arie and Amos Danielli, the contents of which are hereby included by reference.

According to exemplary embodiments of the invention employing this technique, the labeled probe molecule comprises a single strand of DNA with a nucleotide sequence that is complementary to a nucleotide sequence found in a DNA target molecule. The probe molecule, for example a TaqMan® probe, is double labeled with a fluorescent dye and biotin on the same nucleotide at the 5′ end. The dark quencher is connected at the 3′ end. After the probe molecule binds to the target DNA sequence. Taq polymerase activity is used to separate the nucleotides of the probe molecule from the target molecule and from each other. Thus the fluorescent dye molecule, still connected to the biotin, is separated from the dark quencher, but for probe molecules that are not bound to the target DNA sequence, the nucleotides of the probe molecule are not separated from each other, and the fluorescent reporter molecule remains connected in close proximity to the quencher molecule. The biotin is attached to streptavidin-coupled magnetic beads. Further details of how such energy transfer assays are performed are provided, for example, in the paper by Margulis and Danielli, and in the paper by Danielli, Porat, Arie and Ehrlich, both cited above.

In a different kind of assay, a sandwich assay, the probe molecules 104 comprise both sensing probe molecules and reporter probe molecules that bind specifically to the target molecule. For example, the sensing probe molecules, such as a strand of DNA complementary to a part of the sequence of a DNA target molecule, have a molecule of biotin attached to one end, and are attached to a magnetic bead that is coated with avidin, for example. Reporting probe molecules, such as a strand of DNA complementary to a different part of the sequence of the DNA target molecule, have a reporter molecule, such as a fluorescent molecule, attached to one end. Alternatively, the target molecule is an antibody, and sensing probe molecule and reporting probe molecule are two different proteins that bind to different parts of the antibody. Alternatively, the target molecule is a protein, and the sensing probe molecule and reporting probe molecule are two different antibodies that bind to different parts of the protein. When the reporter probe molecule binds to a target molecule that is also bound to a sensing probe molecule, that is bound to a bead, then the fluorescent molecule becomes indirectly bound to the bead, through the target molecule and the two probe molecules, with the two probe molecules forming a “sandwich” around the target molecule. Optionally, the sensing probe molecule binds to the magnetic bead only after it binds to the target molecule, and/or only after the target molecule binds to the reporter probe molecule, and optionally the reporter molecule binds to the target molecule before the target molecule binds to the sensing probe molecule. Reporter probe molecules that do not become bound to the target molecule do not become indirectly bound to the bead, and can be washed away. Consequently the amount of the fluorescent molecules attached to the beads, and the intensity of fluorescent emission, will depend on how many target molecules are bound to the probe molecules, and hence on the concentration of target molecules in the sample. Further details of how sandwich assay are performed are provided, for example, in the paper by Verbarg, Hadass, Olivo and Danielli, and in the paper by Margulis, Ashri and Cohen and Danielli, both cited above.

Instead of or in addition to fluorescent reporter molecules, which are excited by exposure to light of a suitable wavelength, other types of optically emitting reporter molecules are optionally used, for example chemiluminescent molecules that are excited by exposure to a chemical, or electro-chemiluminescent molecules that are excited by an electric current.

Once the magnetic beads have been prepared in container 102 and optionally in other containers, the prepared beads are optionally transferred to a container 112, to read their optical emission signal. Alternatively, their optical emission signal is read in the same container where the beads are prepared, but transferring the beads to a different container has the potential advantage that any reporter molecules that are not bound at least indirectly to the beads will generally no longer be found in the vicinity of the beads when they are transferred to a different container.

In container 112, the beads are suspended in a solution 114, for example a buffer solution, that may also include fluids that were present in container 102, for example fluids, such as blood plasma, whole blood, or urine, that were part of the sample. A magnet 116, optionally a permanent magnet, optionally a small magnet made of a permanent magnet material with high energy product such as NdFEB, is located at or just below the bottom of container 112. Although an electromagnet can also be used, using a permanent magnet has the potential advantage that it can be much smaller than an electromagnet for a moderately strong magnetic field and field gradient. Using such a small magnet may make it possible, for example, to use many small containers for different assays, closely packed together, for example in a well plate, which can allow high throughput for a central laboratory doing a large number of assays. A small magnet may also be more suitable for doing a single assay using a small sample in a small container, for example using a disposable microfluidics cartridge as described below. Magnet 116 attracts the magnetic beads by the magnetic field and magnetic field gradient it produces, and the beads aggregate into a concentrated clump adjacent to the magnet at the bottom of the container. The beads in the clump are then excited to produce optical emission, for example by exposure to a fluorescent excitation light, not shown in FIG. 1, in the case of a fluorescent assay, or by exposure to a chemical in the case of a chemiluminescent assay, or by passing an electric current through them, in the case of an electro-chemiluminescent assay. The optical emission in measured by a light sensor 120, for example a CCD or CMOS camera focused on the clump of beads. The output of the camera is optionally sent, for example, to a computer 122, which can use the measured intensity of optical emission, together with calibration information, to determine the concentration of the target molecules in the sample.

Optionally, a shaker 124, attached to container 112, shakes the container horizontally, either back and forth in one dimension or in a more complicated or random pattern in two dimensions, while the optical emission is being measured by light sensor 120, if sensor 120 is a camera focused on the clump of beads. Alternatively, the shaker is attached to sensor 120, and shakes sensor 120 horizontally, or rapidly changes the direction of its field of view back and forth. The shaking of either the container or the camera, or the relative shaking of both, blurs the image, averaging the optical emission power per area over different parts of the clump that have different surface densities of beads, for example due to statistics of the finite number and size of beads, and/or statistics in the number of reporter molecules per bead. The amplitude of the shaking is optionally such that it blurs the image by at least several times a bead diameter, but not much greater than the clump diameter, and the shaking is optionally rapid enough so that a characteristic period of the shaking is comparable to or shorter than the integration time of light sensor 120 in forming an image. Alternatively, instead of physically shaking the container and/or the light sensor while the optical emission is integrated over time, computer 122 averages the measured optical emission power per area over a number of pixels of the image, optionally covering an area of the image that is at least several times greater in diameter than a bead, but optionally not much greater in diameter than the clump, achieving the same averaging effect numerically. The clump diameter is defined herein as a greatest diameter of an area on the surface where the clump is located, where the density of beads per area is at least 10% of the peak density of beads per area in the clump. Additionally or alternatively, what is stated here about the clump diameter is true for an rms width of the density of beads per area in the clump.

The aggregation of the beads into a clump increases the intensity of optical emission, for a given concentration of target molecules in the sample, which may make the assay more sensitive than if the beads were not aggregated. The inventors have found that having the magnet at or just below the bottom of the container may greatly reduce the time needed for the beads to aggregate into a clump, compared to the time needed to aggregate in assays using magnetic beads where a single magnet is located at the side of the container, or when two electromagnets, alternately magnetized, are located at opposite sides of the container. Since the time needed to aggregate the beads into a clump may be the bottleneck that determines the throughput of such assays, an assay performed using system 100 may have much higher throughput than prior art assays that involve aggregating magnetic beads when their optical emission is measured.

The aggregated clump optionally compromises fewer than 10,000 beads, or between 10,000 and 20,000 beads, or between 20,000 and 50,000 beads, or between 50,000 and 100,000 beads, or between 100,000 and 200,000 beads, or between 200,000 and 500,000 beads, or between 500,000 and 1,000,000 beads, or more than 1,000,000 beads. It is potentially advantageous to use at least enough beads so that the beads are densely packed over a contiguous area, for example approximately a circular area, at the bottom of the container without gaps that are empty of beads. For example, the inventors have found that, using the magnet described below in the section “High throughput optical modulation biosensing system,” placed in a vertical orientation with its tip just below the bottom of the container, gaps are avoided when at least 25,000 M280 beads are used, or when at least 6000 lumavidin beads are used, or when at least 4000 Magplex beads are used. Each bead optionally has a diameter of less than 1 micron, or between 1 and 2 microns, or between 2 and 5 microns, or between 5 and 10 microns, or more than 10 microns. In the tests done by the inventors, described below in the section “High throughput optical modulation biosensing system,” M280 beads with 2.8 micron diameter, and Magplex beads with 5.6 micron diameter, were used. The volume packing fraction of beads in the clump is optionally less than 20%, or between 20% and 30%, or between 30% and 50%, or between 50% and 70%, or more than 70%. The clump optionally covers approximately a circular area on the bottom of the container adjacent to the tip of the magnet, and the diameter of the clump is optionally less than 0.1 mm, or between 0.1 mm and 0.2 mm, or between 0.2 mm and 0.3 mm, or between 0.3 mm and 0.5 mm, or between 0.5 mm and 1 mm, or more than 1 mm. The clump is optionally highest in the center and falls off smoothly to its edge, for example at least approximately like a sphere or a paraboloid, and the height is optionally less than 10% of its diameter, or between 10% and 20% of its diameter, or between 20% and 30% of its diameter, or more than 30% of its diameter. The surface of the clump is optionally oriented normal to the local magnetic force on a bead at each location on the surface, either the force on a single bead ignoring the effect of the other beads on the local magnetic field and field gradient produced by the magnet, or taking into account the effect of the other beads on the local magnetic field and field gradient. Optionally, the effect of the other beads on the local magnetic field and field gradient is modeled by taking into account the volume fraction of the magnetic cores of the beads inside the clump, and the saturation magnetization of the magnetic cores of the beads inside the clump, assuming that the magnetic field produced by the magnet at the location of the clump would, even in the absence of the beads, be much more than enough to saturate the magnetic cores of the beads.

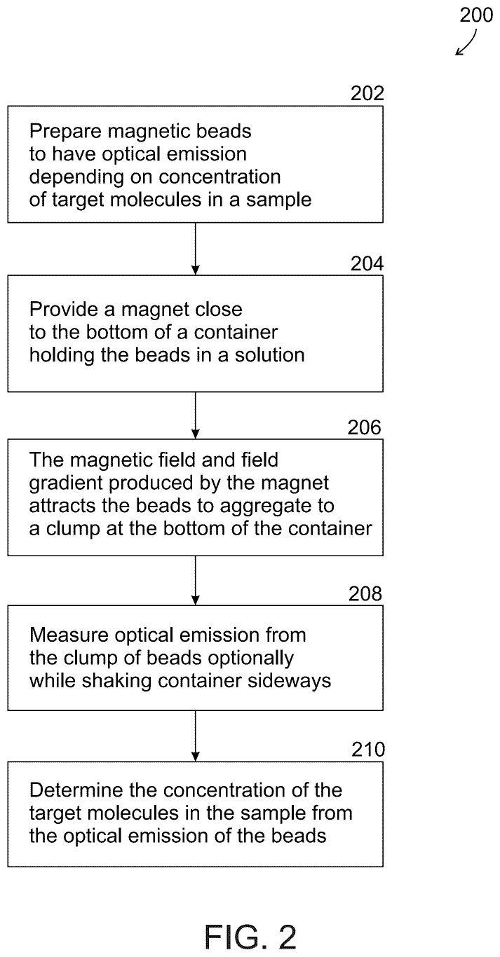

FIG. 2 shows a flowchart 200 for an exemplary method of performing an assay, for example using system 100. At 202, magnetic beads are prepared to have optical emission that depends on the concentration of target molecules in a sample. This can be done, for example, using an energy transfer assay, such as a FRET assay, or a sandwich assay.

At 204, a magnet is provided close to the bottom of a container, such as container 112 in system 100, holding the magnetic beads in a solution. Optionally, this is done by transferring the prepared magnetic beads into container, with the magnet already adjacent to the bottom of the container. Alternatively, the magnetic beads are initially in a container that is not adjacent to a magnet, and the magnet is then brought close to the container. For example, there is a well plate containing a plurality of wells, each containing a solution with magnetic beads prepared with a different sample, and the well plate is moved around so that different wells are sequentially adjacent to a single magnet, the optical emission from the beads in each well measured sequentially when that well is adjacent to the magnet.

At 206, the magnetic field and magnetic field gradient produced by the magnet attracts the beads to aggregate to a clump at the bottom of the container. The magnetic force exerted on a bead is equal to the dot product of the magnetic moment induced in the bead by the magnetic field, and the magnetic field gradient. The magnetic moment is proportional to the external magnetic field for low fields, but is constant for higher fields, when the field inside the bead, which is 3 times the external field for beads with spherical magnetic cores, is greater than the saturation field of the magnetic core. Surprisingly, the inventors have found that the aggregation time, for the same magnetic beads and the same magnet, can be as much as 20 times longer when the magnet is at the side of the container, than when it is below the bottom of the container, with the magnetic force on the beads approximately in the same direction as the gravitational force. In the case described in the Examples section, using M280 magnetic beads, the aggregation time was only 6 seconds when the magnet was below the bottom of the container, but 120 seconds when the magnet was just outside the side of the container. In both these cases, the aggregation time dominated the time needed to measure the optical emission of the beads, so it is the aggregation time that sets the throughput rate for the assays.

At 208, the optical emission signal from the clump of beads is measured, when the emission is excited, for example by illuminating the beads with a fluorescent excitation light, in the case of a fluorescent assay. Optionally, especially if the optical emission is measured by a camera that forms an image of the clump of beads, the optical emission is measured while shaking the container horizontally, along one axis or in two orthogonal directions, with a characteristic period much shorter than the integration time of the measurement, and over a distance much greater than a bead diameter but not greater than the clump diameter, averaging over variations, for example statistical variations, in the optical emission power over the surface of the clump. Alternatively or additionally, the same averaging effect is achieved by shaking the light detector horizontally or in orientation, and/or is achieved numerically by averaging the optical emission power over a number of pixels covering an area of the image. At 210, the concentration of target molecules in the sample is determined from the optical emission signal of the beads, by making use of a suitable calibration factor.

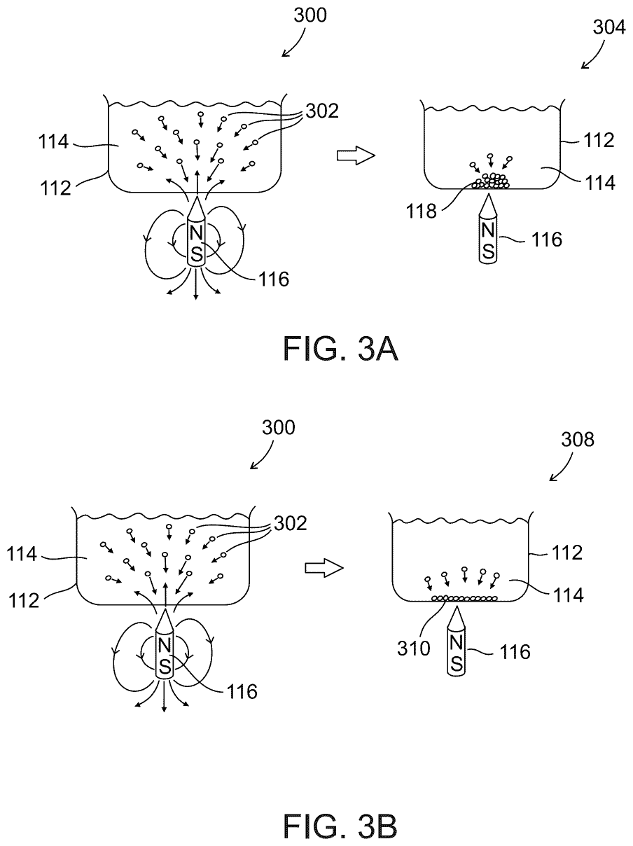

FIG. 3A shows a schematic view 300 of magnetic beads 302 in container 112, containing solution 114, and with magnet 116 just below the bottom of the container, initially, shortly after the beads have been introduced into the container, or shortly after the magnet has been brought close to the container. Some of the field lines of magnet 116 are shown, near the bottom of the container, and the velocities of magnetic beads 302, suspended in the solution, are shown as arrows going towards the tip of the magnet. In view 304 of container 112, shown later in time, most of the beads have aggregated to form clump 118 at the bottom of the container, adjacent to the tip of the magnet, and a few remaining beads are shown approaching the clump.

FIG. 3B schematically shows a view 306 of an alternative configuration, in which the tip of magnet 116 is further below the bottom of container 112, than in FIG. 3A. For example, in FIG. 3A the tip of the magnet is touching the bottom outer surface of container 112, or is within a distance much less than the diameter of the magnet from the bottom outer surface of the container, or from the bottom inner surface of the container, while in FIG. 3B, the tip of the magnet is at a distance comparable to the diameter of the magnet, or greater than the diameter of the magnet, below the inner or outer bottom surface of the container. For example, in FIG. 3B, the tip of the magnet is at least 2 mm below the inner bottom surface of the container, or at least 3 mm the inner bottom surface, or at least 5 mm below the inner bottom surface, or at least 10 mm below the inner bottom surface. Having the tip of the magnet further below the bottom of the container in FIG. 3B makes the magnetic field inside the container, especially near the bottom, less directed radially toward a point just above the tip of the magnet, and more directly vertically, and more uniform in direction over the bottom of the container, than in FIG. 3A. As a result, as schematically shown in view 308, instead of the magnetic beads forming a dome-shaped clump like clump 118 in FIG. 3A, just above the tip of the magnet, the beads form a wider, flatter clump 310, spread out over a wider area than clump 118, on the bottom of the container. With the magnet far enough below the bottom of the container, depending on the number of beads present, the beads may form a wide single layer only one bead thick, but densely and fairly uniformly packed within that layer. This has the potential advantage that the fluorescent emission signal from all the beads can be detected, with none of the beads, or very few of the beads, hidden behind other beads, which can result in a higher signal to noise ratio, for the same number of beads. If there are enough beads present, and if the magnet is close enough to the bottom of the container so that the beads are densely and uniformly packed within the area taken up by the beads, then the fluorescent emission intensity within that area may depend only on the average number of fluorescent reporter molecules per bead, and may be insensitive to random variations in where the beads end up at the bottom of the container, especially if the fluorescent emission intensity is averaged or integrated over the whole area that the beads take up at the bottom of the container. In these circumstances, the fluorescent emission signal may be a more accurate measure of the concentration of the target molecule in the sample, than would be the case for the configuration of FIG. 3A. But it is potentially advantageous if the magnet isn't too far below the bottom of the container, otherwise the beads may be so spread out over the bottom of the container that they are not densely packed in the area they take up, and then it may be more difficult to estimate the fluorescent emission power per bead. Also, if the magnet is too far below the bottom of the container, then the magnetic force on the beads will be greatly reduced, and the aggregation time may be much longer.

Similar results can be achieved with a wider magnet, or with a more rounded tip, for example a parabolic or circular tip, or with no tip on the top of the cylindrical magnet, which will also make the magnetic field have less of a radial component directed to a point just above the tip, and have a more uniform and vertical orientation over the bottom of the container. Alternatively, the magnet has a plurality of tips located at different points below the bottom of the container, and/or there are a plurality of magnets each with its own tip, at least approximately parallel to each other with their north poles oriented in the same direction, located at different points below the bottom of the container. Those configurations may produce a plurality of clumps of beads, one clump above each tip, and the total area of the clumps, for a given number of beads, may be greater than the area of clump 118 in FIG. 3A. Depending on the number of beads and the lateral spacing of the tips, and distance of the tips below the bottom of the container, the different clumps might or might not overlap, and the clumps might comprise a layer only one bead thick, or they might be thicker than one bead, and if the clumps comprise a layer only one bead thick, the beads might or might not be densely packed within that layer. Optionally, in any of these cases, the fluorescent emission signal is measured by measuring the intensity of emission as a function of position on the bottom of the container, and averaging or integrating over the area where beads are present. This configuration may produce some or all of the potential advantages of the configuration shown in FIG. 3B.

The inventors have tried using a configuration similar to that shown in FIG. 3B, but with the magnet outside a side wall of the container, and have found that it does not produce a densely packed layer, one bead thick, on the wall of the container, but produces an area of beads that is broken up, with some parts of the area not having any beads.

### Estimate of Magnetic Forces and Aggregation Times

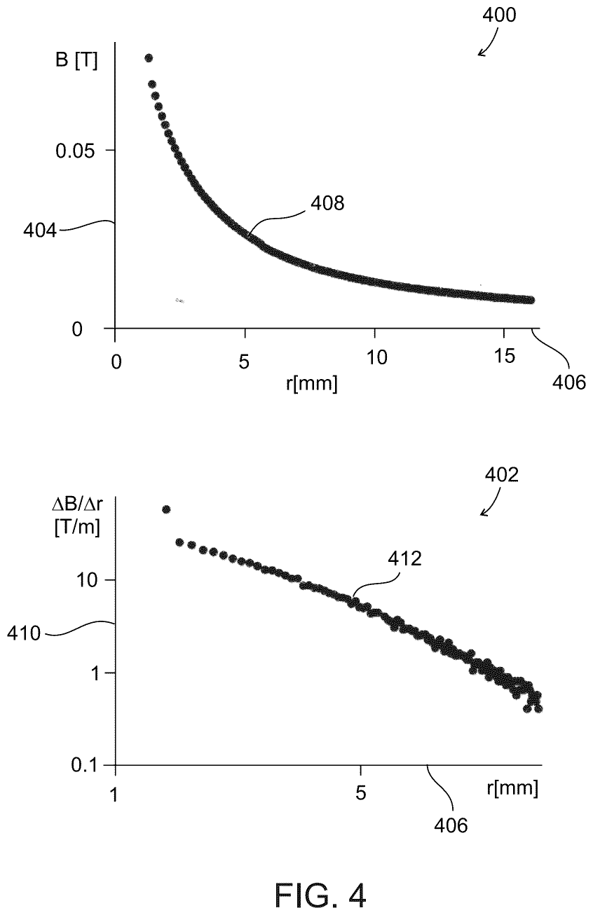

FIG. 4 shows a plot 400 showing the magnetic field B in teslas, as a function of distance r, in millimeters, above the tip of the magnet, and a plot 402 showing the gradient dB/dr in teslas per meters, as a function of distance r, in millimeters above the tip of the magnet, for the rare earth magnet used in the experiments described in the Examples section. It should be understood that directly above the tip of the magnet, along the cylindrical symmetry axis of the magnet, dB/dr is the magnitude of the gradient VB, for reasons of symmetry. The vertical axis 404 on plot 400 shows B in teslas on a linear scale, the horizontal axis 406 shows r in millimeters on a linear scale, and data points 408 show B as a function of r, as measured with a gaussmeter. On plot 402, vertical axis 410 shows dB/dr is teslas per meter on a logarithmic scale, and the horizontal axis 406 shows r in millimeters on a linear scale, and data points 412 shows dB/dr as a function of r, calculated from data points 408. For the smallest value of r shown, which is 1.27 mm, B is about 0.075 tesla, and dB/dr is about 50 tesla per meter. For r less than about 1.8 mm, B is greater than 0.05 tesla, and the magnetic cores of M280 beads are magnetically saturated. For r small compared to the diameter of the magnet, which is 6 mm, but large compared to the radius of curvature of the sharp iron tip of the magnet, B may increase roughly like 1/r, and dB/dr may increase roughly like 1/r², and B may be as high as 0.3 teslas at the bottom of the container, which is only 0.175 mm from the tip of the magnet, and the field gradient may be as high as 1500 teslas per meter, and the magnetic force on a bead is very great. But further away from the bottom of the container, the field and field gradient are much lower, and the magnetic force on a bead is much smaller.

The values of B and VB, or dB/dr, plotted in FIG. 4, can be used to estimate the magnetic force on the magnetic beads used in the tests described in the Examples section, and to estimate the time that a magnetic bead, pulled by the magnetic force, would take to reach the point at the bottom of the container directly over the tip of the magnet, from different distances r above the bottom of the container. The magnetic force on a magnetic bead is given by m_{bead}·∇Bₑₓₜ where m_{bead} is the magnetic moment of the magnetic core of the bead, and ∇Bₑₓₜ is the external magnetic field gradient. The magnetic core of a magnetic assay bead is generally paramagnetic or superparamagnetic, and does not remain permanently magnetized, but has a magnetic moment only when it is in an external magnetic field Bₑₓₜ. The magnetic moment is in the direction of Bₑₓₜ, and has a magnitude equal to the magnetization M of the core integrated over the volume of the core. When Bₑₓₜ is not too high, and if the core is assumed to be a sphere with permeability μmuch greater than the vacuum permeability μ₀=4π10⁻⁷, then the field inside the core will be uniform at 3Bₑₓₜ in the direction of the external field, and the magnetization M of the core will be uniform at 2Bₑₓₜ/μ₀ in the direction of the external field. When the magnetic field inside the core starts to approach the saturation field Bₛₐₜ of the magnetic core material, which occurs when Bₑₓₜ is about Bₛₐₜ/3, then the marginal permeability dB/dH of the core material starts to fall to (o, and any additional external field does not further increase the magnetization M of the core material, which remains at about 2Bₛₐₜ/3μ₀, and the magnetic moment m_{bead} of the core of the bead will have a constant value mₛₐₜ for Bₑₓₜ>>Bₛₐₜ/3. In general, m_{bead}≈mₛₐₜ×min(1, 3Bₑₓₜ/Bₛₐₜ). For the M-280 superparamagnetic beads used for the tests described below in the Examples section, the magnetic cores are made of a ferrite material that has Bₛₐₜ≈0.15 tesla, and the saturation magnetic moment mₛₐₜ of a bead is 1.6×10⁻¹³ A·m². For Bₑₓₜ<0.05 tesla, the magnetic moment of a bead will be 1.6×10⁻¹³ A·m²(Bₑₓₜ/0.05 T).

For the permanent magnets used for the tests described in the Examples section, the field Bₑₓₜ, and the field gradient ∇Bₑₓₜ are plotted in FIG. 4 as a function of distance r directly above the tip of the magnet. Both Bₑₓₜ and ∇Bₑₓₜ are vertical, directly above the tip. In those tests, the tip of the magnet was touching the outer surface of the bottom of the container, which was 0.65 mm thick, so the bottom inside surface of the container, where the clump forms, was at r=0.65 mm. Bₑₓₜ falls to 0.05 tesla at r≈1.9 mm, so for r<1.9 mm, the bead is saturated and its magnetic moment is 1.6×10⁻¹³ A·m², while for r>1.9 mm the bead is not saturated and its magnetic moment is 3.2×10⁻¹² Bₑₓₜ A·m², where Ben is in tesla.

At r=1.4 mm, using the values plotted in FIG. 4, we find a magnetic force F_{mag}=9×10⁻¹² newtons on the bead. If that force is balanced by a viscous drag force in water, then the terminal velocity, which will be reached before the bead has travelled very far, will be V=F_{mag}/6πηR, where n=1.0016×10⁻³ Pa·s is the viscosity of water at room temperature, and R=1.4×10⁻⁶ meters is the radius of the bead. The instantaneous velocity of the bead at any location will be very close to the velocity at which the viscous drag force balances the magnetic force at that location, and as the bead moves into different locations where the magnetic force is different, the velocity of the bead will quickly adjust itself so that the viscous drag force continues to balance the local magnetic force. So the velocity of a bead at a distance of 1.4 mm above the tip will be 0.3 mm/s. Since the magnetic force, and hence the velocity, will quickly increase as the bead gets closer to the magnet, the time needed for the bead to reach the bottom of the container will likely be on the order of 1 or 2 seconds. But further from the magnet, the magnetic force will be much lower, and the time needed to reach the bottom of the container will be much greater. For example, at r=3 mm above the magnet, the magnetic force will be about 3.5×10⁻¹² newtons, the velocity of the bead will be about 0.12 mm/s, and the bead may take on the order of 10 seconds to reach the bottom of the container. At r=7 mm above the magnet, the magnetic force will be about 4×10⁻¹⁴ newtons, which is approximately equal to the net gravitational force (the gravitational force minus the buoyant force) on the bead, which has a mass of 1.6×10⁻¹⁴ kg, and a diameter of 2.8 μm. The velocity of the bead will then be about 0.1 mm per minute, and the bead will take on the order of 103 seconds to reach the bottom of the container.

These estimates suggest the importance of having a relatively low depth of solution, as well as having a container that does not extend too far laterally, for example no more than 3 mm in depth and no more than 3 mm in any direction laterally from the tip of the magnet, in order for the beads to aggregate rapidly in the field of the magnet, for example in less than 10 or 20 seconds. But it should be noted that these numbers for the travel time of individual beads are only rough estimates, and collective effects, where beads attract each other and possibly move in each other's wake, may also play an important role in aggregating the beads into a clump. Also, if the solution includes significant quantities of substances with high viscosity, such as blood plasma, then the terminal velocity of the particles at a given distance above the magnet may be much lower, and the aggregation time may be much longer.

The aggregation time was measured for three different configurations. A cuvette, 0.4 mm wide, 8 mm high, and 70 mm long, and filled with water, was used with an electromagnet, with a parabolic pole piece adjacent to the outside of one of the wide faces of the cuvette, centered at a point half-way up the 8 mm height, and near the front. With this configuration, the beads took 120 seconds to aggregate.

The aggregation time was also measured in the well of a well plate. The well was circular, 6 mm in diameter and filled 2 mm deep with water. The magnet was the permanent magnet with a pointed tip described in the Example section, located directly under the center of the bottom of the well. The beads took 6 seconds to aggregate.

The aggregation time was also measured in a microfluidics cartridge, in a chamber 18 mm long, 4 mm wide, and 0.14 mm deep, filled with water. A permanent magnet with a pointed tip, the one described in the Example section, was located directly under the center of the bottom of the chamber. The aggregation time was 6 seconds.

### Fluorescent Assays

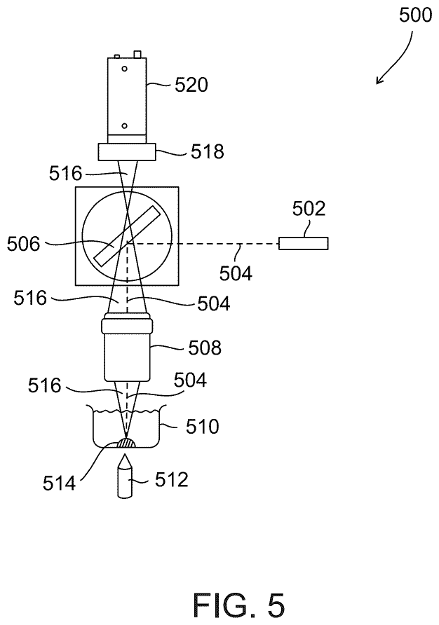

FIG. 5 schematically shows a system 500 used for an assay where the optical emission is fluorescent emission, which is excited by a fluorescent excitation light beam, according to an exemplary embodiment of the invention. A light source 502, such as a laser, generates light of a wavelength or range of wavelengths that excited fluorescent emission by the fluorescent reporter molecules that are bound to the magnetic beads. For example, 532 nm is a commonly used wavelength for excitation light in assays using fluorescent emission. Any light source that produces light of suitable wavelengths, that can be focused on the clump of magnetic beads, can be used to produce the excitation light beam. Light source 502 produces an excitation light beam 504, which reflects from a dichroic mirror 506. Dichroic mirror 506 largely reflects light with the wavelength of the excitation light, but largely transmits light of the longer wavelength of the fluorescent emission from the beads, which makes it possible to use some of the same optical components both for illuminating the beads with the excitation light beam, and for detecting the fluorescent emission from the beads. After reflecting from the dichroic mirror, light beam 504 passes through a lens 508, for example a 10× microscope eyepiece. Beam 504 then enters container 510, which has a magnet 512 just below the bottom of the container, which has attracted the magnetic beads to form a clump 514. Optionally, magnet 512 has a sharp tip that produces a sharp magnetic field gradient that causes the magnetic beads to aggregate into a clump.

Optionally, magnet 512 has a plurality of sharp tips, or there are a plurality of magnets each with a sharp tip, that cause the magnetic beads to aggregrate into a plurality of clumps, and light beam 504 illuminates all or some of the clumps simultaneously. Alternatively, light beam 504 illuminates each clump or some of the clumps sequentially, and the fluorescent emission is measured from each of those clumps sequentially, and measured fluorescent emission is summed or averaged over the different clumps. Having more than one clump has the potential advantage that the total surface area of the clumps may be greater, for a given total number of beads, than if there is only one clump, potentially producing a stronger emission signal.

Lens 508 focuses beam 504 down to a small spot that illuminates clump 514 and its beads. For example, the spot is smaller than 0.1 mm, or between 0.1 mm and 0.2 mm, or between 0.2 mm and 0.3 mm, or between 0.3 mm and 0.5 mm, or bigger than 0.5 mm.

Optionally, the spot is smaller than the diameter of the clump, so the light produced by light source 502 is used efficiently to produce fluorescent emission, and to keep the fluorescent emission high compared to any background signal generated by beam 504. But optionally the spot is not too much smaller than the clump, so that the signal strength will not be too low, and the noise level will not be too high, and hence the signal to noise ratio will not be too low. Having a higher signal to noise level may allow the assay to be more accurate and to have greater sensitivity. For a given total excitation light power illuminating the clump, concentrating the power into a smaller spot may cause saturation of the fluorescent emission, or may cause photobleaching, and using a larger spot size may allow higher total excitation power to be used, and hence higher fluorescent emission power, while avoiding saturation or photobleaching. Using too small a spot size may also increase the noise level in the fluorescent emission, due to statistical fluctuations in the number of fluorescent reporter molecules that fall within the spot, especially near the sensitivity limit of the assay. Moving a narrow spot across the clump, instead of keeping it focused on one part of the clump, may reduce these statistical fluctuations. Optionally, the spot covers at least 20% of the area of the clump, or at least 30% of the area, or between 30% and 50% of the area, or between 50% and 70% of the area, or the spot covers all of the area of the clump. Optionally at least 50% of the power of excitation beam 504 illuminates the clump, or at least 70% of the power of excitation beam 504 illuminates the clump, or all of the power of excitation beam 504 illuminates the clump, ignoring any power of beam 504 that is scattered or absorbed before reaching the clump.

Excitation beam 504 excites the emission of fluorescent light rays 516. The fluorescent light is emitted in all directions, but FIG. 5 shows a fan of light rays 516 that reach and enter lens 508. Light rays 516 go through lens 508, and pass through dichroic mirror 506 with relatively little reflection, because they have longer wavelength than excitation light beam 504. For example, for fluorescence that is excited by light of 532 nm, the fluorescent emission light may have wavelength predominantly in a range between 550 nm and 590 nm. Finally, light rays 516 pass through a filter 518, that admits much of the fluorescent emission light but substantially blocks any scattered light from excitation light beam 504, and enters a light sensor 520, for example a CMOS camera or a CCD that records an image of the clump of beads and the surrounding area on the bottom of container 510. Alternatively, light sensor 520 is just a single sensor, which measures the total fluorescent emission from the beads in the clump, for example, without forming any image.

When beam 504 passes through the solution in container 510, on the way to clump 514, it produces some background light at the same wavelength range as the fluorescent emission from the beads. For example, some of this background light comes from Raman scattering of light beam 504 in the water of the solution, and some of the background light may come from a small quantity of the fluorescent reporter molecules that may be dissolved in the solution in the container. This background light adds to the fluorescent emission signal from the beads, and may cause errors in the concentration of the target molecule in the sample, found by the assay.

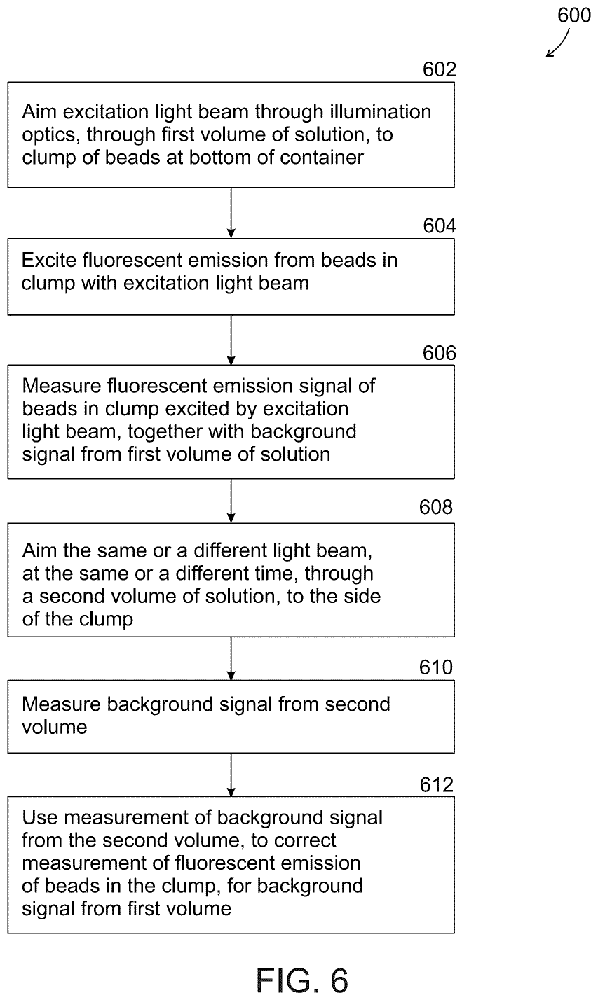

FIG. 6 shows a flowchart 600, for a method of estimating the size of the background signal, and correcting the fluorescent emission signal for the error caused by the background signal, according to an exemplary embodiment of the invention. This is done, for example, by aiming the light beam, or a different light beam of about the same wavelength and intensity, to the side of the clump of beads, through nearly the same depth of water as the light beam that illuminates the beads. When the light beam is aimed to the side of the clump in this way, it produces only a background signal, which may be close in magnitude to the background signal that comes from the solution in front of the beads. Knowing the size of that background signal, it can be subtracted from the measured fluorescent emission signal, to find the actual signal of the fluorescent emission, to good approximation.

At 602, an excitation light beam is aimed through the illumination optics, through a first volume of the solution, to illuminate the clump of beads at the bottom of the container. At 604, fluorescent emission from the beads in the clump is excited by the excitation light beam. At 606, the fluorescent emission signal of the beads in the clump, together with a background signal from the first volume of the solution in front of the beads, is measured. At 608, the same excitation light beam, or a different light beam with similar power and wavelength, is aimed through a second volume of the solution, to the side of the clump, missing the clump. This can be done alternately with illuminating the beads, for example by moving the beam back and forth, or by alternately turning each beam on and off. Or it can be done simultaneously with illuminating the beads, for example by using two different light sources, or by using a single light source and splitting its beam into two beams aimed in slightly different directions, one illuminating the clump of beads, and the other passing to the side of the clump of beads. A potential advantage of illuminating the beads and the second volume alternately, instead of simultaneously, is that the much brighter fluorescent emission light from the beads will not scatter and appear as it if is coming from the second volume, and cause an error in measuring the background emission from the second volume. A potential advantage of illuminating the beads and the second volume simultaneously, is that it may take less time to make the two measurements than if one measurement is done after the other.

At 610, the background signal is measured from the second volume. At 612, the background signal from the second volume is used to correct the measured fluorescent emission signal for the background signal from the first volume. For example, if the light beam that illuminates the clump of beads is the same light beam that passes through the second volume, and if it is passes through substantially the same depth of solution when it is illuminated the clump of beads as when it is passing to the side of the clump of beads, for example within 10%, or within 5%, or within 2%, then the first volume and the second volume will be substantially the same, and the background emission from the first volume is expected to be substantially the same as the background emission from the second volume. The first volume and second volume will also be substantially the same if different beams are used, but they have substantially the same beam width, for example within 10%, or within 5%, or within 2%. If the two beams also have substantially the same wavelength or distribution of wavelengths, so that they are expected to produce substantially the same measured background signal relative to their brightness, for example within 10% or within 5% or within 2%, due to Raman scattering and dissolved fluorescent molecules in the solution, and they also have substantially the same brightness, for example within 10% or within 5% or within 2%, then again the background emission from the first volume is expected to be substantially the same as the background emission from the second volume. In this case, the background signal from the second volume may be subtracted from the measured fluorescent emission from the beads, to obtain the actual fluorescent emission from the beads. Alternatively, if there are significant differences between the light beam that illuminates the beads through the first volume, and the light beam that passes through the second volume, then the known differences between the two light beams are optionally used to estimate an expected ratio between the background signal from the first volume and the background signal from the second volume. That ratio, and the measured background signal from the second volume, may then be used to find an expected background signal for the first volume, which can be used to correct the measured fluorescent emission from the beads.

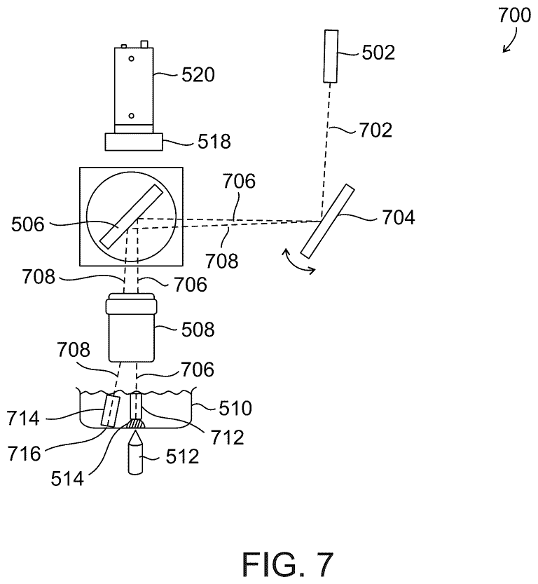

FIG. 7 schematically shows an exemplary system 700 that can be used to perform the method of flowchart 600, using a single beam that swings back and forth between illuminating the clump of beads through the first volume, and passing through the second volume to the side of the clump of beads. Light source 502 produces a light beam 702 suitable to use as fluorescent excitation light for the beads. Light beam 702 reflects off rotating mirror 704, which swings back and forth between two orientations, optionally spending about half of the time in each orientation, and transitioning from one orientation to the other rather quickly. At a first orientation, light beam 702, after reflecting from mirror 704, follows the path labeled 706, and will be referred to as light beam 706. At the second orientation, light beam 702, reflecting from mirror 704, follows the path labeled 708, and will be referred to as light beam 708. Both light beam 706, and light beam 708, at different times, reflect from dichroic mirror 506, pass through lens 508, and into container 510, with magnet 512 beneath just below the bottom of the container. Light beam 706 passes through first volume 712 of the solution in container 510, and illuminates clump 514. Light beam 708 passes though second volume 714 of the solution in container 510, and illuminates an empty position 716 at the bottom of container 510. Fluorescent emission from the beads in clump 514, as well as background emission, due for example to Raman scattering from water in the solution, and fluorescent emission from any loose fluorescent molecules dissolved in the solution, passes through lens 508, passes through dichroic mirror 506, passes through filter 518, and is received and recorded by light sensor or camera 520, similar to what is shown in FIG. 5 for light rays 516, but these emission light rays are not shown in FIG. 7, for clarity. Optionally, camera or light sensor 520 records light from the clump of beads and the first volume, and light from the second volume, on different pixels of an image, so the two signals can be distinguished that way. Alternatively, light sensor 520 has only a single pixel that records both the fluorescent emission from the beads, and the background light from volumes one and two, but the two signals are distinguished because light beam 706 illuminates the clump of beads, and the first volume, at different times than light beam 708 illuminates the second volume.

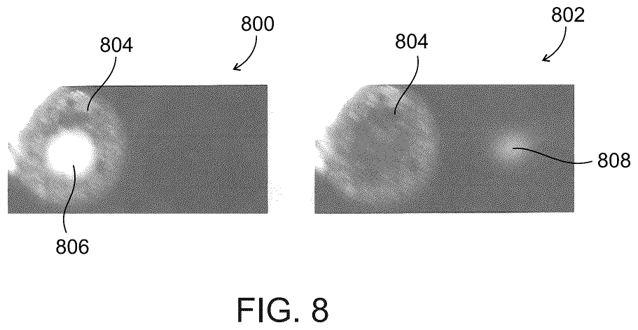

FIG. 8 shows images of the bottom of the container, recorded by the camera, at two different times. Image 800 was made when the light beam was illuminating the clump of beads, and the first volume. Image 802 was made the when the light beam was passing through the second volume, to the right of the clump. In image 800, a bright spot 806 shows the fluorescent emission from the beads in the clump, coming from the portion of the surface of the clump that is illuminated by the excitation light beam. Because the light has reached the camera only after passing through the dichroic mirror, which for the most part only transmits light at the wavelengths of the fluorescent emission, and reflects light at the wavelengths of the excitation light, and because the light reaches the camera only after passing through filter 518, which blocks of the excitation light wavelengths and transmits light of the fluorescent emission wavelengths, spot 806 in image 800 represents essentially only fluorescent emission from the beads excited by the excitation light beam, and not excitation light reflecting from the beads. In image 802, there is a much dimmer spot 808, that represents light, at the fluorescent emission wavelengths, coming from the second volume, due to Raman scattering of the excitation light, and due to fluorescent emission from any dissolved fluorescent reporter molecules in the second volume. Some of the excitation light may reflect from the bottom of the container at point 716 in FIG. 7, or may scatter from particles in the water without any change in wavelength, but that light is not visible in image 802, because it is the wrong wavelength and has not reached the camera. Because the second volume is nearly the same size and shape as the first volume, the background signal from the second volume is expected to be very close in amplitude to the background signal from the first volume, and can be simply subtracted from the measured fluorescent emission signal to obtain the true fluorescent emission signal from the beads in the clump.

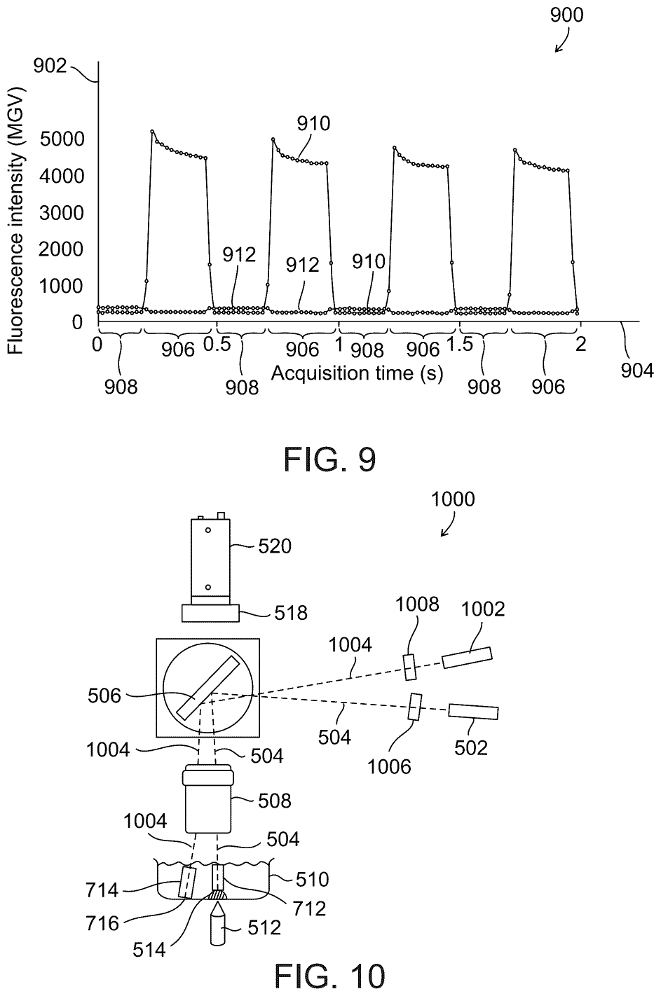

FIG. 9 shows a plot 900 of the fluorescent emission signal measured from the beads in the clump, and the background signal from the second volume. The signal amplitude is shown by a vertical axis 902 and time in seconds is shown on a horizontal axis 904. The signal amplitude is given in units of Mean Gray Value, which are arbitrary units proportional to the emission power. In FIG. 9, the Mean Gray Value is normalized to the emission power per area from the beads with no attached fluorescent reporter molecules, with the emission coming from the autofluorescence of the M-280 beads, illuminated by the same 532 nm excitation light. At time intervals 906, the excitation light beam is illuminating the clump of beads, through the first volume, and at time intervals 908, the excitation light beam is passing through the second volume to the side of the clump of beads. The amplitude of the fluorescent emission signal from the beads, as a function of time, is curve 910, and the background signal from the second volume, as a function of time, is curve 912. Curve 910 is essentially zero at time intervals 906, when the beads are not illuminated, and curve 912 is essentially zero at time intervals 908, when the second volume is not illuminated. During time intervals 906, when the beads are illuminated, curve 910 is at a high value, corresponding to the fluorescent emission from the beads, plus the background signal from the first volume. Curve 910 drops slightly during each time interval 908, due to some photobleaching of the fluorescent reporter molecules bound to the beads. Either the initial value of curve 910 during each time interval 906, or the final value, or an average value during each time interval 906, could be used to measure the fluorescent emission from the beads, as long as the same rule is used when calibrating the assay. The value of curve 912 that occurs during time intervals 908 gives the background signal from the second volume. Subtracting this background signal from the measured fluorescent emission signal gives the corrected fluorescent emission from the beads.

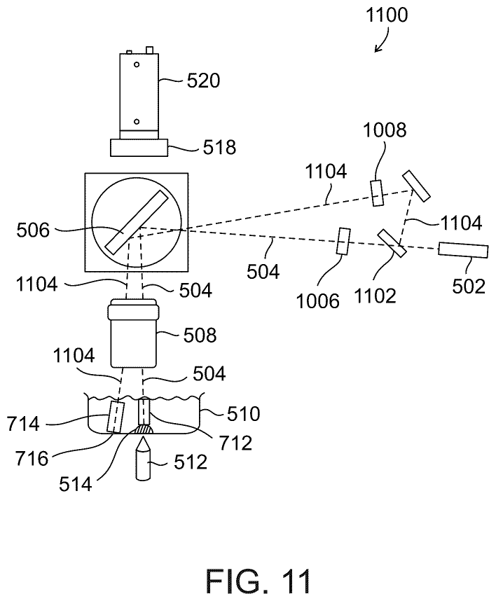

FIGS. 10 and 11 show two other systems that can be used to perform the method of flowchart 600. In system 1000, instead of having a single excitation light beam swing back and forth between the clump of beads and the second volume, there are two light beams produced by two different light sources aimed in slightly different directions. Light source 502 produces light beam 504, which reflects from dichroic mirror 506, passes through lens 508, and illuminates clump 514 through first volume 712. A second light source 1002 produces a second light beam 1004, which also reflects from dichroic mirror 506 and passes through lens 508, and then passes through second volume 714 in the solution in container 510, passing to the side of clump 514. As in FIG. 7, the fluorescent emission from the clump of beads, and the background signal from the second volume, pass through lens 508, through dichroic mirror 506, through filter 518, and into camera 520, and they can be used in the same way to correct the fluorescent emission of the beads for background from the first volume. Optionally, light beams 504 and 1104 are identical or nearly identical in power, width, and wavelength, so they produce nearly the same background signal from the first and second volumes. If there are known differences between the two beams, then the differences can be used to multiply the background signal from the second volume by a correction factor, before subtracting it from the measured fluorescent emission signal from the second volume. Optionally, shutters 1006 and 1008 are used respectively to shut off beam 504 when beam 1004 is propagating, and to shut off beam 1004 when beam 504 is propagating. This has the potential advantage that each signal (fluorescent emission from the beads plus the first background signal, and the second background signal) can each be measured at different times, without any interference from the other. But measuring both signals at the same time has the potential advantage that the measurements could be accomplished more quickly.

In FIG. 11, a system 1100 has a single light source 502 that produces a light beam. A beam splitter 1102, put in the path of the light beam from light source 502, divides the beam into a beam 504 and a beam 1104, aimed in slightly different directions. As in FIG. 7 and FIG. 10, the two beams both reflect from dichroic mirror 506, go through lens 508, and enter container 510. Beam 504 illuminates the clump of beads 514 through first volume 712, and beam 1104 passes through the second volume 714. As in FIG. 7 and FIG. 10, the two signals, from the beads plus the first volume, and from the second volume, are both measured by camera 520, and the background signal from the second volume can be used to correct the fluorescent emission from the beads. As in FIG. 7 and FIG. 10, shutters 1006 and 1008 alternately shut off each beam. Optionally, beam splitter 1102 splits the beam from light source 502 into two beams of nearly equal power. Alternatively, the power ratio between the two beams is measured, and the background signal from the second volume is corrected by that power ratio, before subtracting it from the measured fluorescent emission signal from the beads in the clump.

### Well Plate Systems

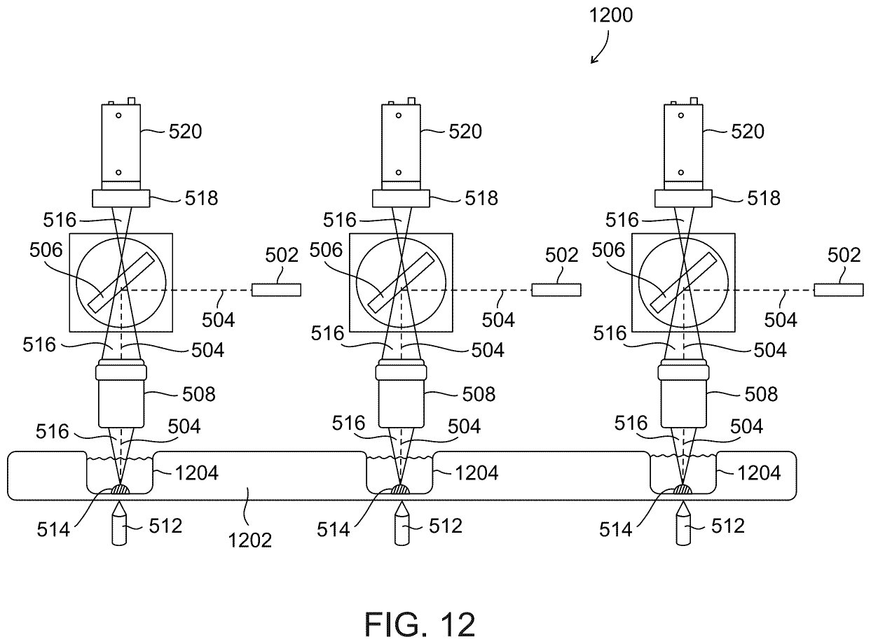

FIG. 12 schematically shows a system 1200 for performing assays using a well plate 1202, with a plurality of wells 1204 each for performing a different assay, according to an exemplary embodiment of the invention. For each well there is, for example, a detection and illumination sub-system like the system shown in FIG. 5, and there is a magnet 512 just below each well which aggregates the beads into a clump 514, as in FIG. 5. Alternatively, the sub-system for each well looks like any other assay system for a single assay using magnetic beads aggregated at the bottom of the well by a magnet, for example like the systems shown in FIG. 7, 10 or 11. Because the entire system is duplicated for each well, any or all the assays are optionally performed in parallel, potentially saving time.

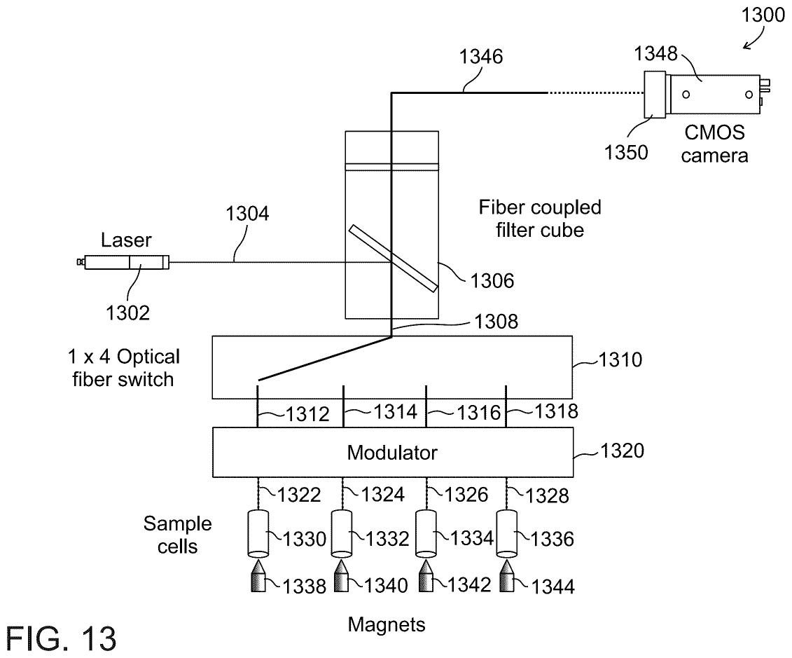

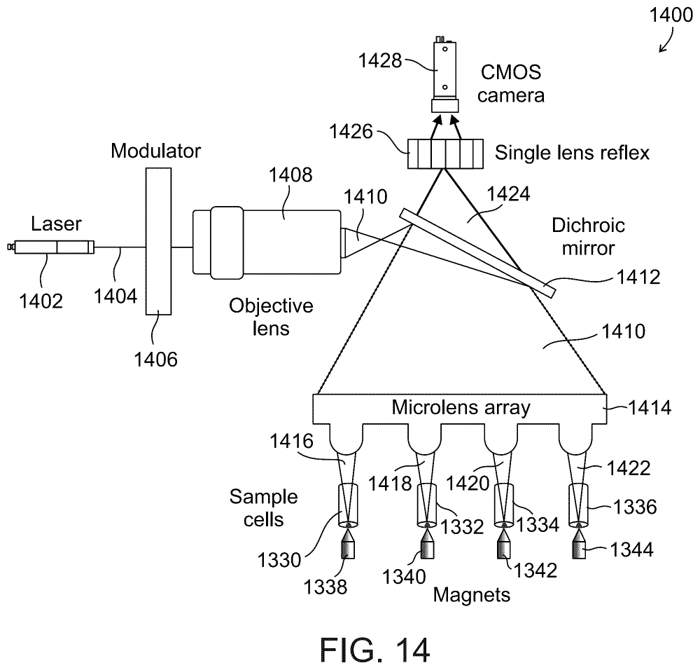

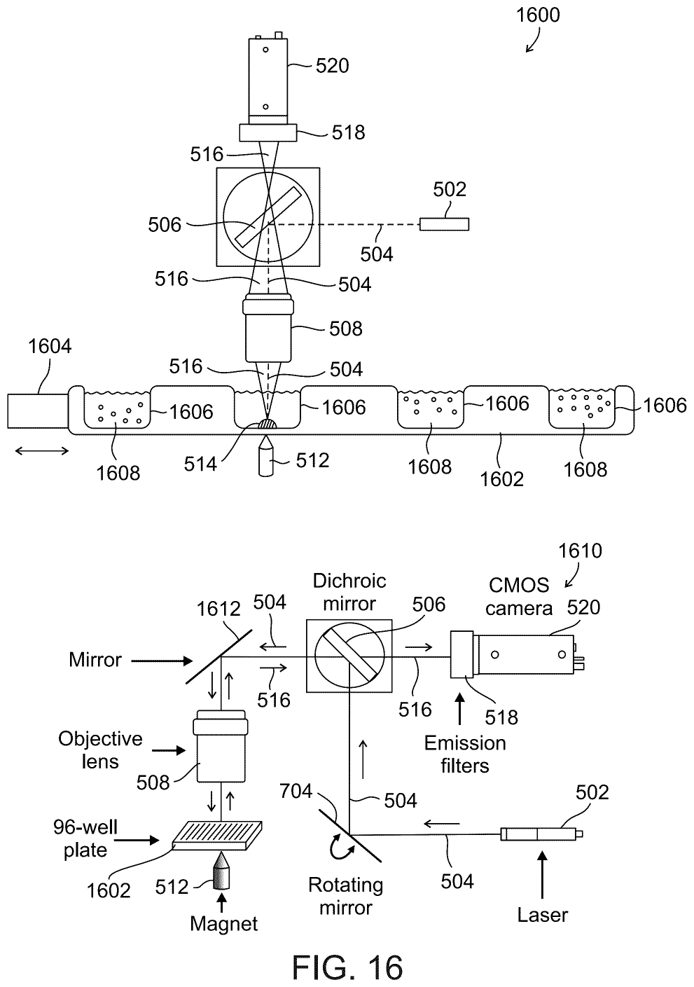

FIGS. 13, 14 and 16 show alternative systems for performing assays using a well-plate, or a set of containers, in which the illumination and detection sub-system is not duplicated for each well, but there is only one illumination and detection sub-system, and optionally only one magnet, which, in FIGS. 13 and 16 are used sequentially to read the fluorescent emission from the magnetic beads in each well. In FIG. 14, the light source illuminates all of the wells simultaneously, so all of the wells can optionally be read in parallel. Having a single illumination and detection sub-system for all of the wells has the potential advantage, over system 1200, that the illumination and detection sub-systems do not have to be as narrow as the space above each well, in a standard well plate, or it is not necessary to use a non-standard well plate where the wells are much further apart than in a standard well-plate, and it is likely to be less expensive to use a single illumination and detection sub-system than to duplicate it for each well. It should be understood that it is also possible to use a combination of parallel and sequential reading of wells, for example each row of a well-plate can have its own illumination and detection sub-system and magnet, and within each row each well is read sequentially, while this is done in parallel for the different rows.

FIG. 13 schematically shows a system 1300 for reading the fluorescent emission from a clump of beads at the bottom of each of a plurality of sample cells or wells, sequentially using a single light source 1302 and a single camera, according to an exemplary embodiment of the invention. It has the potential advantage, over the system of FIG. 16 described below, that it has no moving parts, with the possible exception of some small motion of the modulator depending on the type of modulator used. The light source, for example a laser, produces a light beam 1304 of suitable wavelength for fluorescent excitation of the beads. In a fiber coupled filter cube 1306, light beam 1304 is coupled into an optical fiber 1308, which carries the light to an optical fiber switch 1310. The switch can be controlled to couple optical fiber 1308 to any one of a plurality of optical fibers, four optical fibers 1312, 1314, 1316 and 1318 in the case illustrated in FIG. 13, one optical fiber for each well. An example of a suitable fiber optic switch is described, for example, by Q. Xiang, B. Xu, and D. Li, “Miniature real-time PCR on chip with multi-channel fiber optical fluorescence detection module,” Biomed Microdevices, DOI 10.1007/s10544-007-9048-4, Springer Science+Business Media LLC. 2007.

The four optical fibers are coupled to a modulator 1320, which modulates the direction of the light beam emerging from each fiber, so that it alternates between illuminating the clump of beads at the bottom of the well passing through a first volume of solution above the clump, and passing through a second volume of the solution to the side of the clump of beads. Light beams 1322, 1324, 1326 and 1328, emerging from modulator 1320, respectively enter wells 1330, 1332, 1334 and 1336, which respectively have magnets 1338, 1340, 1342 and 1344 beneath them, that each aggregate the magnetic beads to a clump at the bottom of that well.

Light is emitted from each well, the fluorescent emission from the beads and background emission from the first volume of solution when the beam is illuminating the clump of beads, and background emission from the second volume of solution when the beam is passing to the side of the clump. The emitted light is received by the optical fiber for that well, and coupled back into fiber 1308, and into optical fiber 1346, which brings the light to camera or light sensor 1348, which has a filter 1350 in front of it that excludes light that is not of the wavelength range of fluorescent emission from the beads, and in particular blocks any stray light from light source 1302, at the excitation wavelength. Optionally, if only single optical fibers carry the fluorescent emission light to the camera or light sensor, then no image of the bottom of the well is formed, but the camera or light sensor only measures an integrated or average emission from the bottom of the well that is being illuminated. Alternatively, instead of single optical fibers 1308, 1312, 1314, 1316 and 1318, optical cables are used, each comprising an array of optical fibers, and each with a suitable image-forming optical element such as a lens at its end, such as the optical cables used in an endoscope, and sensor 1348 is a camera, and an image of the bottom of the well that is being illuminated is formed, and conveyed by the optical cables to the camera.

After the fluorescent emission signal is measured for each well, and recorded as a function of time as the excitation beam swings back and forth, the optical fiber switch switches to the fiber for the next well, and its fluorescent emission signal is recorded as a function of time as the excitation beam swings back and forth, until all the wells have been recorded. For each well, the signal as a function of time can be used to correct the measured fluorescent emission signal for the background signal, as described above for FIGS. 7, 8, and 9, but optionally using only the integrated emission from each well, rather than forming an image of the bottom of the well. That can be done, because at any given time, the light beam is illuminating only the clump of beads, or only passing through the second volume of solution to the side of the clump, and not both.

FIG. 14 shows a system 1400 for reading the fluorescent emission from each of a plurality of wells or sample cells, using a microlens array, according to an exemplary embodiment of the invention. A description of a suitable microlens array, and of other elements used in system 1400, for imaging a sample that produces fluorescent emission, is given, for example, by Antony Orth and Kenneth B. Crozier, “High throughput multi-channel fluorescence microscopy with microlens arrays,” Optics Express 22, 18101-18112 (2014). System 1400 has the potential advantage, over the systems in FIG. 13 and below in FIG. 16, that the fluorescent emission from all of the wells can be read in parallel, saving time. It also has the potential advantage over the system of FIG. 16 that it has no moving parts, with the possible exception of small motion in the modulator. A light source 1402 produces a beam 1404 of excitation light. The light beam passes through a modulator 1406, which slightly varies its direction back and forth, so that, when it reaches the wells, it will alternate between illuminating the clump of beads, and passing to the side of the clump of beads and producing only a background signal. The light beam then passes through an objective lens 1408, where it emerges as light rays 1410, which come to a focus and spread out. Light rays 1410 reflect from a dichroic mirror 1412, and illuminate a microlens array 1414. A plurality of sample cells, or wells in a well plate, schematically illustrated in FIG. 14 as four sample cells 1330, 1332, 1334 and 1336, are each illuminated by a different microlens in the microlens array. Each sample cell or well has a solution with magnetic beads in it, and has a magnet beneath it, illustrated in FIG. 14 by magnets 1338, 1340, 1342, and 1344. Each magnet aggregates the magnetic beads in the sample cell or well into a clump at the bottom of the sample cell or well, adjacent to the tip of the magnet. The microlenses, and sample cells or wells, and magnets, are arranged so that, for each sample cell or well, a portion of light rays 1410 is concentrated by one of the microlenses onto the clump at the bottom of that sample cell or well. For example, as shown schematically in FIG. 14, light rays 1416 are concentrated on the clump at the bottom of sample cell 1330, light rays 1418 are concentrated on the clump at the bottom of sample cell 1332, light rays 1420 are concentrated on the clump at the bottom of sample cell 1334, and light rays 1422 are concentrated on the clump at the bottom of sample cell. This condition pertains when modulator 1406 is set so that beam 1404 from the light source is aimed so that light rays 1416, 1418, 1420 and 1422 will be concentrated on the clumps, passing through a first volume of the solution in each sample cell. However, when modulator 1406 is set so that beam 1404 is aimed in a slightly different direction, which is not shown in FIG. 14, then light rays 1416, 1418, 1420 and 1422 will respectively miss the clumps in their sample cells, and instead be directed to the side of the clumps, passing through a second volume of solution in each sample cell. Rays of fluorescent emission light and background light from each sample cell or well are collected by the microlens array as light rays 1424, and are transmitted through dichroic mirror 1412, reaching a single lens reflex lens 1426, which forms an image of the sample cells or wells for camera 1428. The image can be used to measure the fluorescent emission from the beads in the clump, plus the first volume background emission, simultaneously for all the sample cells, when the light rays are illuminating the clump in each sample cell, through the first volume in each sample cell. And the image can be used to measure the second volume background emission from each cell simultaneously, when the light rays are passing through the second volume and missing the clump, in each sample cell. Alternatively, for example if the sample cells and their magnets are not aligned precisely properly with respect to the microlens array, then modulator 1406 is optionally set to modulate the direction of the beam slowly over a wider range of directions, instead only in two directions, so the excitation light can reach the clump in all sample cells at some time, even if not at the same time for all sample cells. Then the clumps in different sample cells may be illuminated at different times, and the second volumes of different sample cells may be illuminated at different times, but the fluorescent emission from the clump in each sample cell, and the background emission from the second volume in each background cell, may be found from images recorded by the camera over a period of time. This may take longer, and may require some additional image processing software to recognize when the clump is illuminated in each sample cell and when the second volume is illuminated, but has the potential advantage that the sample cells and magnets need not be aligned so precisely with the microlens array.

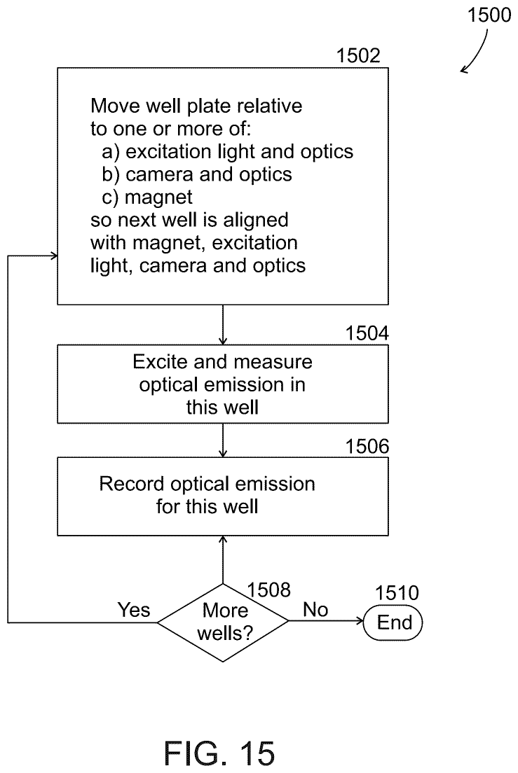

FIG. 15 shows a flowchart 1500, that describes a method of reading the fluorescent emission from multiple wells in a well plate, for example using the system of FIG. 16, according to an exemplary embodiment of the invention. In method 1500, different wells are read sequentially, but the system of FIG. 16 has the potential advantage over the systems of FIGS. 13 and 14 that the excitation beam illuminates the wells directly, without the need for fiber optic switches or microlens arrays that might require careful alignment with the wells. The system of FIG. 16 has the potential advantage over the system of FIG. 12 that some or all of the excitation light and optics, the light detector and optics, and the magnet, need not be duplicated for all the wells, but there can be only one of them, used sequentially for different wells, making the system potentially less expensive.

At 1502, the well plate is moved relative to one or more of the excitation light source and optics, the light detector (or camera) and optics, and the magnet, so that the next well to be read is aligned with a magnet, an excitation light source and optics, and a light detector (or camera) and optics. This can be done either by moving the well plate, and/or by moving the excitation light source and optics, the light detector and optics, and/or the magnet, depending on which of them are being used for more than one well. If the light source, light detector, or camera, or magnet is being used only for one well, with each well having its own, then those elements need not be moved between reading one well and the next. In practice, it may be most practical for each well to have its own magnet, since the magnets are small and relatively inexpensive, but to have a single light source, and a single light detector, both with associated optics, that is used for all the wells in sequence, since those elements are likely to be more expensive and to take up more room. In the system shown in FIG. 16, there is also only a single magnet that is used sequentially for all the wells, as well as a single light source and optics, and a single camera and optics.

In some embodiments of the invention, instead of moving a light source or camera around from one well to another, or moving a light detector or camera from one well to another, the light source remains in place, or the light detector remains in place, and one or more optical elements, such as mirrors, are moved, in order to move an excitation beam of the light source from being directed to one well to being directed to another well, or in order for the light detector or camera to receive light from a different well.

At 1504, the optical emission, for example fluorescent emission, is excited in the beads in the well that is being read, and the emission is measured. Optionally, any of the methods described in FIGS. 6-11, for correcting the emission measurement of the beads for the background signal, are used for measuring the emission.

At 1506, the optical emission for this well is recorded. At 1508, it is decided whether there are any more wells to be read. If not, the method ends at 1510. If there are more wells to be read, then control returns to 1502, and the next well is aligned with the light source and optics, the light detector and optics, and/or the magnet, whichever of those elements are being used for more than one well.

FIG. 16 schematically shows a system 1600 for sequentially reading fluorescent emission from magnetic beads in each of a plurality of wells in a well plate 1602, for example using the method of FIG. 15. Well plate 1602 has a motor 1604 that moves the well plate horizontally, optionally in two horizontal dimensions if the wells are arranged in two horizontal dimensions. For example motor 1604 is optionally an x-y positioner, for example using piezoelectric motors. Well plate 1602 has a plurality of wells 1606, illustrated in FIG. 16 as four wells. Each well contains a solution 1608 with magnet beads from a different assay, that emit fluorescent emission that is to be measured in order to obtain the results of the assay. The optical elements and magnet of system 1600 are configured like those of system 500 in FIG. 5. A light source 502 produces an excitation light beam 504 that reflects from a dichroic mirror 506, and through a lens 508. A magnet 512, positioned just below the bottom of the well that is currently being read, aggregates the magnetic beads in that well into a clump 514 at the bottom of the well. Lens 508 focuses light beam 504 on clump 514. Fluorescent emission light rays 516, emitted by the magnetic beads when they are excited by the excitation light, are collected by lens 508, and pass through dichroic mirror 506, because they have a longer wavelength than excitation light beam 504. Light rays 516 pass through filter 518, which filters out stray shorter wavelength light from the light source, and are recorded by camera 520. Optionally, camera 520 records an image of the bottom of the well that is being read, including the clump, or a portion of the clump, that is illuminated by light beam 504 and is emitting fluorescent emission light. Alternatively, camera 520, or a light sensor that is used instead of camera 520, only records an integrated or average emission of light from the bottom of the well, or from a field of view that includes the portion of the clump that is emitting fluorescent light.

In some embodiments of the invention, instead of arranging the optical elements of system 1600 like those of system 500 in FIG. 5, the optical elements of system 1600 are arranged like the systems shown in FIG. 7, 10 or 11, and described also in FIGS. 6, 8 and 9. In those embodiments, a measurement is made of the fluorescent emission of beads in the clump, together with background emission from a first volume of the solution that light beam 504 passes through on the way to the clump, and another measurement is made of background emission produced by a beam of excitation light passing through a second volume of the solution to the side of the clump. The background emission from the second volume is then used to correct the measurement of fluorescent emission from the clump, for the background emission from the first volume. For example, system 1610, shown schematically in FIG. 16, is similar to system 1600, but with the optical elements like those of system 700 in FIG. 7, including a rotating mirror 704 that can change the direction of beam 504 from being aimed at the clump of beads in each well, to being aimed through the second volume of solution to the side of the clump in each well. In system 1610, well plate 1602 is shown in a perspective view, with 96 wells arranged in a two-dimensional array, rather than in a cross-sectional view with only four wells, as shown schematically for system 1600.

The inventors estimate that with a system like system 1610, it should be possible, reading the fluorescent emission from each of the 96 wells sequentially, to read the entire well plate in 10 minutes. This estimate is based on the observation that, for a container similar in size and shape to a well in a standard 96-well plate, for example if each well is 6 mm in diameter with a 2 mm depth of solution, and with a neodymium permanent magnet with a sharp tip located directly beneath the well oriented vertically, the magnetic beads in the well can be aggregated into a clump in 6 seconds, and the time needed to measure the fluorescent emission from the clump of beads is much less than 6 seconds, and the time needed to move the well plate so that the next well can be read is also much less than 6 seconds.

In some embodiments of the invention, instead of having only one magnet 512 that is successively located between each well as it is read, each well has its own magnet that remains below it when the well plate moves. This configuration has the potential advantage that in all of the wells clumps of magnetic beads aggregate at the bottom, when the magnetic beads and solution are first introduced, and it is not necessary, when reading each well, to wait for the clump of magnetic beads to aggregate, which may greatly decrease the time needed to read a well plate. Some potential advantages of having only one magnet, that is successively brought beneath each well before it is read, are that a standard off-the-shelf well plate can be used, and that it would be less expensive to have only one magnet than to have a separate magnet for each well, especially if the magnets are permanently fixed to the well plate, and if the well plate is disposable. Also, if the one magnet is permanently well aligned with the optical elements of system 1600, then it may not be necessary to position the well plate so precisely when each well is read, since the clump will always be located where light beam 504 is aimed.

### Microfluidics Cartridge

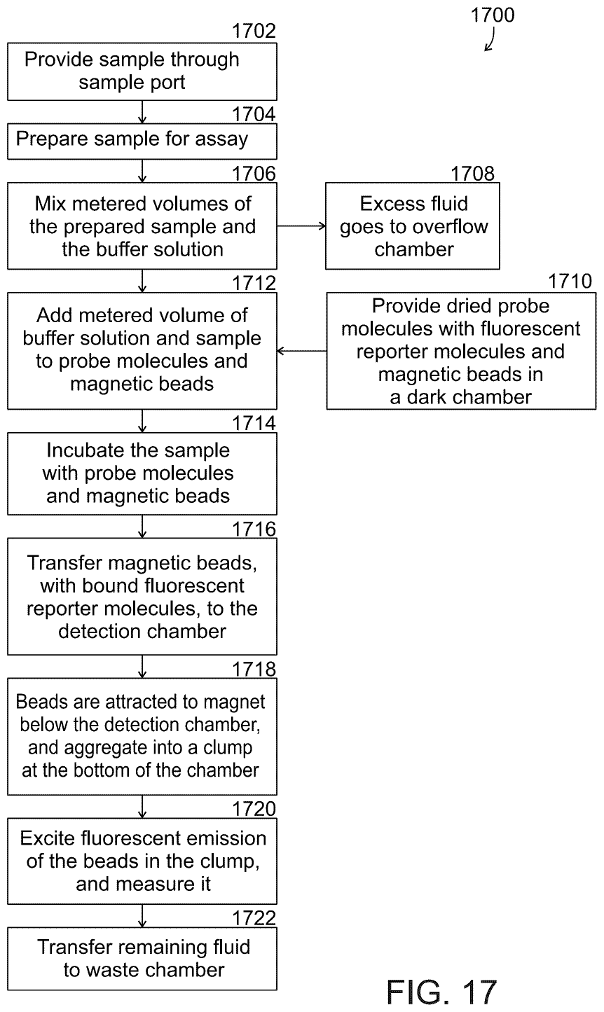

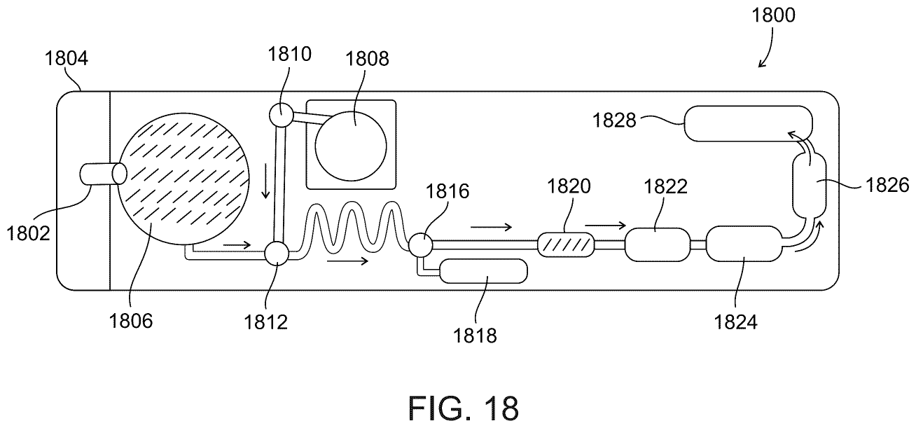

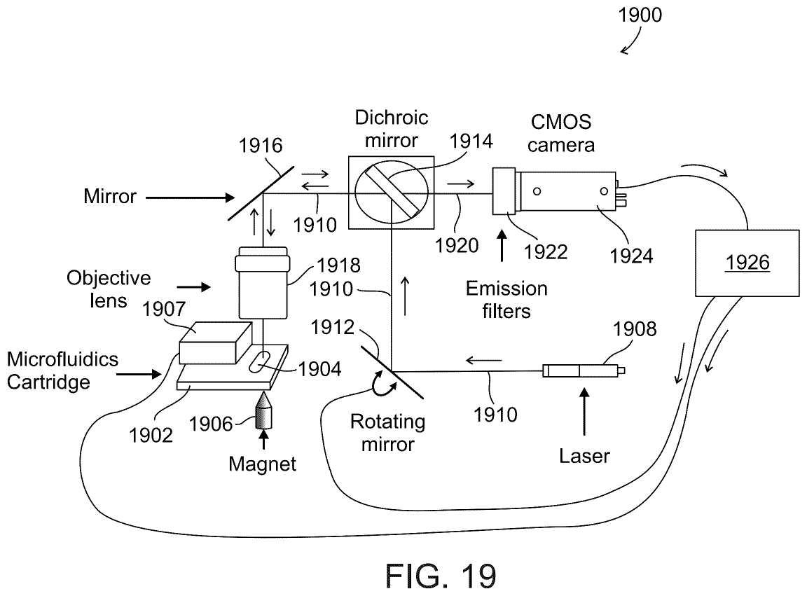

FIG. 17 shows a flowchart 1700 for performing an assay using a microfluidics cartridge, for example a disposable cartridge, for example made of plastic, where the assay involves measuring optical emission, including fluorescent emission, or other optical emission excited by excitation light, from magnetic beads that have been aggregated by a magnet to form a clump at the bottom of a container. FIG. 18 shows an exemplary microfluids cartridge that can be used for such a method, and FIG. 19 shows an exemplary system that can be used to perform an assay with such a microfluidics cartridge using such a method, including a light source for exciting the emission, a camera or light detector for measuring the emission, associated optics, and a magnet for aggregating the magnetic beads into a clump.

At 1702, a biological sample is provided through a sample port of the microfluidics cartridge. At 1704, the sample is prepared for the assay. For example, if the sample is whole blood, and the target molecule that is being assayed is a component of the blood plasma, for example an antibody, then preparing the sample might comprise separating the blood plasma from the other blood components, and using only the plasma to perform the assay. Alternatively, depending on the nature of the sample and the target molecule, the whole sample may be used for the assay, with no need to perform any special preparation of the sample.

At 1706, a metered volume of the prepared sample is optionally mixed with a metered volume of a buffer solution, and at 1708, any excess fluid, beyond the metered volumes, is sent to an overflow chamber. Alternatively, the sample itself may provide enough liquid to perform the assay, and no added buffer solution is needed or used. In that case, the solution referred to above, in the descriptions of performing the assay and reading the optical emission of the beads, may mean a liquid part of the sample, for example blood, or blood plasma, or urine, or saliva, or a liquid from a plant.

At 1710, probe molecules with fluorescent reporter molecules, optionally in dry form, and magnetic beads, are provided, stored in a chamber in the microfluidics cartridge. Alternatively the probe molecules and/or other reagents are stored in wet form, for example together with the buffer solution, or in a separate chamber filled with a liquid in the microfluidics cartridge. Optionally, the chamber is dark inside, protected from light, in order to avoid photobleaching the fluorescent reporter molecules while the cartridge is being stored, before use. The probe molecules and fluorescent reporter molecules may be the molecules needed for any of the types of assays described above, for example they may be the molecules needed for energy transfer assays, such as FRET assays, or the molecules needed for sandwich assays. Other reagents needed for the assay may also be included, in the same chamber or in a different chamber, to be introduced, by opening or closing one or more valves, manually or automatically, together with the other molecules, or at different times, as needed for the assay. For example, if the assay includes PCR, as energy transfer assays sometimes do, in order to separate a part of the probe molecule with an attached fluorescent reporter molecule from a part of the probe molecule with an attached quencher, then agents used for the PCR are introduced at an appropriate time, and heat is applied and removed at an appropriate time to perform the PCR. Whatever type of assay is done, the probe molecules, at least some of them, are optionally already attached to the magnetic beads when they are stored in the chamber, before performing the assay, or alternatively the probe molecules are attached to the magnetic beads only later, as part of the assay.

At 1712, to a metered volume of the buffer solution and sample are added the dried probe molecules, with fluorescent reporter molecules, any other reagents, and the magnetic beads, together or at different times, as appropriate for the type of assay being performed. At 1714, the sample, including any buffer solution, the probe molecules with the fluorescent molecules, and the magnetic beads, are incubated together for a period of time, for example for 10 minutes, or different components of these are incubated together at different times. The end result of the incubation is that fluorescent reporter molecules will be bound to the magnetic beads at a concentration that depends on the concentration of a target molecule in the sample.

At 1716, the magnetic beads, with their bound fluorescent reporter molecules, are transferred to a detection chamber. At 1718, the magnetic beads are attracted to a magnet located below the bottom of the detection chamber, by the magnetic field and magnetic field gradient produced in the detection chamber by the magnet, and the beads aggregate into a clump on the bottom of the detection chamber, adjacent to the tip of the magnet, where it comes closest to the detection chamber.

At 1720, fluorescent emission is excited from the beads, for example in response to illuminating the beads with fluorescent excitation light, and the fluorescent emission is measured. The measured fluorescent emission is used to find the concentration of the target molecule in the sample, using any of the methods described above.

At 1722, the remaining contents of the detection chamber, optionally including the magnetic beads, are optionally transferred to a waste chamber. If the microfluidics cartridge is disposable, it may then be disposed of, preferably in a safe way.

FIG. 18 schematically shows a microfluidics cartridge 1800, as seen from above, that may be suitable for performing method 1700 of FIG. 17. The sample is introduced into the cartridge through an input port 1802. Optionally, a cap 1804 covers the input port until the cartridge is ready to be used, to avoid any contamination of it by target molecules, and to avoid the introduction of any moisture that may degrade some of the stored dry reagents. The sample is prepared for the assay in a chamber 1806, for example chamber 1806 comprises a filter that separates blood plasma from a sample of whole blood, if the target molecule is expected to be present only in the plasma component. A chamber 1808, for example a blister chamber, contains a buffer solution that will be mixed with the sample to perform the assay. The buffer solution, or a portion of it, is emptied from chamber 1808, for example by pressing on the blister, while a meter 1810 only allows a specified volume of buffer solution to flow past it. The prepared sample, or a portion of it, is emptied from chamber 1806, while a meter 1812 allows only a specified volume of the prepared sample, and the buffer solution, to flow past it, into pipe 1814. A meter 1816 allows only a specified volume of the buffer solution with the prepared sample to flow past it, while the remainder of the buffer solution with the prepared sample flows into an overflow chamber 1818. The buffer solution with the prepared sample optionally flows through another filter 1820, and then through a chamber 1822 in which are stored dry reagents, such as probe molecules with fluorescent reporter molecules, and magnetic beads. The buffer solution, sample, reagents and magnetic beads are then transferred to incubation chamber 1824, where they incubate for a specified time, for example 10 minutes. Optionally the sample, reagents and beads are not all mixed together in chamber 1822 at the same time, but some components of them are mixed together before other components, at specified times, optionally introduced from different chambers, depending on how the assay works. Optionally the buffer solution and prepared sample are caused to move around inside microfluidics cartridge 1800, in the manner described, by pressing on different chambers and/or tubes from the outside, for example with valves to ensure that the buffer solution and prepared sample flow in a correct direction. Pressing on a chamber can increase fluid pressure inside the chamber, producing a pressure gradient between that chamber and a tube or another chamber connected to it, which will cause fluid to flow out of the chamber that is being pressed on. A similar effect can be produced by depressing a plunger from outside the cartridge that reduces the volume inside a chamber. Additionally or alternatively, a plunger can be drawn up from outside the cartridge, decreasing the pressure inside a chamber, and producing a pressure gradient that draws fluid into that chamber from an adjacent tube or chamber. Optionally, there are mechanisms, for example knobs in the form of screw heads, for opening and closing valves at different times, from outside the cartridge. Additionally or alternatively, there are one-way valves that ensure that fluid flows in a correct direction, without any need to actively open or close the valves. Optionally, the amount and timing of the applied pressure changes, and the timing of opening and closing of any valves, is controlled automatically by a machine that the cartridge is inserted into, and the only things that are done manually are to introduce the sample into the cartridge through the sample port, and to place the cartridge in the machine. Optionally, the same machine includes a light source, a camera, associated optics, and magnet, for reading the fluorescent emission from the beads, as will be described in FIG. 19.

When the sample, buffer solution, reagents and beads are finished incubating in chamber 1824, the contents of chamber 1824, including the beads, are transferred to a detection chamber 1826. As will be described in FIG. 19, a magnet below chamber 1826 aggregates the beads into a clump at the bottom of chamber 1826, and an excitation light beam, introduced from above chamber 1826, illuminates the clump of beads, which emit fluorescent light. The fluorescent emission is detected and measured by a camera or other light detector, with suitable optics. When the measurement is complete, the contents of detection chamber 1826 are optionally emptied into a waste chamber 1828, and if the cartridge is disposable, it is then optionally disposed of.

FIG. 19 shows a system 1900 for reading the fluorescent emission from magnet beads aggregated into a clump at the bottom of a chamber in a microfluidics cartridge 1902, such as cartridge 1800 in FIG. 18, using the method of flowchart 1700 in FIG. 17. The optical system for exciting and reading fluorescent emission is similar to the system shown in FIG. 7, but alternatively could be like any of the other optical systems described herein, for example in FIGS. 5, 10 and 11. Microfluidics cartridge 1902 has a detection chamber 1904, similar to detection chamber 1826 in FIG. 18. A magnet 1906 is located directly below the bottom of detection chamber 1904, and causes the magnet beads in chamber 1904 to aggregate into a clump at the bottom of the chamber. A controller 1907, located above the microfluids cartridge, includes actuators that press on one of more chamber or tubes of the cartridge, and/or that depress or withdraw plungers connected to one of more chambers or tubes of the cartridge, and/or open or close valves in the cartridge, to cause the sample, buffer solution, magnetic beads and reagents to move properly from one chamber to another, in the proper order and with the proper timing, to perform the assay, as described for FIGS. 17 and 18. Optionally, controller 1907 is configured or programmed to perform all these steps automatically, and a user of system 1900 need only put a sample into the cartridge through its input port, and insert the cartridge into a machine which automatically performs all of the other steps described in FIGS. 17 and 18, including reading the fluorescent emission once the magnetic beads are in detection chamber 1904 and have been aggregated into a clump. To read the fluorescent emission, a light source 1908 produces a beam 1910 of excitation light, which reflects from rotating mirror 1912, from dichroic mirror 1914, and optionally from another mirror 1916, into lens 1918. Lens 1918 focuses the excitation light beam on the clump of magnetic beads at the bottom of detection chamber 1904 in cartridge 1902. The magnetic beads, illuminated by the excitation light, cmit fluorescent light rays 1920, which are collected by lens 1918, reflected from mirror 1916, and pass through dichroic mirror 1914, because they have a longer wavelength than the excitation light beam. Light rays 1920 are then filtered by filter 1922, which blocks any stray light of the excitation light wavelength, and enter camera or light sensor 1924. Optionally light rays 1920 produce an image of the bottom of the detection chamber, including the clump of magnetic beads, a part of the clump which is illuminated by the excitation light and producing fluorescent emission, and at least a part of the bottom of the detection chamber to the side of the clump. Alternatively, camera or light sensor 1924, does not form an image of the bottom of the detection chamber, but only records an integrated or average intensity of light at a fluorescent emission wavelength or range of wavelengths coming from a field of view in the detection chamber. Rotating mirror 1912, at one or more time intervals, rotates by an amount and in a direction that directs excitation light beam in a slight different direction, so it misses the clump of magnetic beads, and passes to the side of the clump, through a second volume of the solution in the detection chamber, as described above for FIGS. 6 and 7. During these time intervals, camera 1924 measures the background emission from the second volume, while during one or more other time intervals, when light beam 1910 is directed at the clump through a first volume of the solution in the detection chamber, camera 1924 measures the fluorescent emission of the beads plus background emission from the first volume. Camera 1924 sends data of these two measurements to a computer 1926. Computer 1926 optionally calculates a correction to the measured fluorescent emission for the first volume background emission, using the measured second volume background emission to estimate an expected contribution from the first volume background emission, as described above for FIGS. 6 and 7. Optionally, as shown in FIG. 19, computer 1926, or one or more other computers or controllers that it is optionally in communication with, controls rotating mirror 1912, and controller 1907, so that it knows, for example, when the magnetic beads are expected to be in detection chamber 1904, when they have had time to aggregate into a clump, and when excitation beam 1910 is aimed at the clump and when it is aimed to the side of the clump. Alternatively or additionally, computer 1926 figures out this information open loop, from the data that it receives from camera 1924.

### High Throughput Optical Modulation Biosensing System

Experiments were done with a high throughput optical modulation biosensing (OMB) system, with a configuration similar to system 700 in FIG. 7 with a rotating mirror, and more specifically like system 1610 in FIG. 16, since a well plate was used. The experiments measured the sensitivity and dynamic range of the system, and the reproducibility of its results.

The high throughput OMB system uses a 532 nm laser diode module 502 (CPS532, ThorLabs Inc.), working at 0.25 mW with a beam 504 that is 3.5 mm in diameter. Beam 504 is reshaped by a pair of plano-convex lenses (not shown in FIG. 16) with focal lengths of 200 mm and 100 mm (ThorLabs Inc.), redirected by a rotating scanning mirror 704 (GVS211, Thorlabs Inc.), and diverted by a dichroic mirror 506 (BrightLine Di02-R532, Semrock). Then, beam 504 is redirected vertically by a mirror 1612 (BB1-E02-Ø1″ Broadband Dielectric Mirror, ThorLabs Inc.) and focused by an infinity-corrected objective lens 508 (MY10X-803-10X Mitutoyo Plan Apochromatic Objective, 0.28 NA, 34 mm WD, ThorLabs Inc.) to a 150 μm diameter beam spot onto a standard 96-well plate 1602 (Bio-Plex Pro™ Flat Bottom Plates #171025001, BioRad) that contains the samples of biological reagents. The magnetic beads are aggregated and immobilized to the detection spot by a cylindrical permanent magnet 512 (axially magnetized. neodymium N42, 6.35×25.4 mm, D4X0, K&J Magnetics, Inc., USA) with a conic tip (6.35 mm in diameter and 8.62 mm high, Hiperco 50, Ed Fagan, UK). The emitted fluorescence light rays 516 are collected by the same objective lens 508, redirected horizontally by the same mirror 1612, pass through dichroic mirror 506 and two emission filters 518 (FF01-575/25, Semrock), and captured by a CMOS camera 520 (GS3-U3-23S6M, FLIR Systems, Inc.).

The lateral movement of the laser beam is generated by connecting the scanning galvo mirror's motor to a power source (GPS011, Thorlabs Inc. Newton, NJ, USA) and a function generator (AFG3022B, Tektronix, Beaverton, OR, USA). The mirror is rotated in a square waveform at a frequency of 2 Hz with an amplitude of 225 mV, which moves the laser beam back and forth over a distance of 500 μm on the well's bottom, between the center of clump 514 and location 716 in FIG. 7. To prevent overheating, the motor is attached to a heat sink (GHS003, Thorlabs Inc, Newton, New Jersey, USA).

Camera 520 obtains an image of the bottom of the well, with a field of view that includes both the clump of beads aggregated by the magnet, and the location to the side of the clump to which the excitation light beam is directed when it misses the clump. For each measurement, 50 frames were acquired over a period of one second. Each frame was divided into two areas; the side with the clump of beads and the side without the beads. To identify the position of the beam in each area, the frames were cropped and thresholded. A binary mask of the beam's position in each area was applied to all the frames. The mean gray value (MGV) in each frame was calculated and plotted as a function of time on a graph, producing a result similar to what is shown in FIG. 9. The mean gray value plotted in FIG. 9 was found by subtracting the mean gray value on the side of the image without the beads, averaged over the pixels where the beam was directed there, from the mean gray value on the side with the beads, averaged over the pixels where the beam was directed there. The result was normalized to the fluorescent emission from beads without attached fluorescent reporter molecules, which is due to the autofluorescence of the bead material.

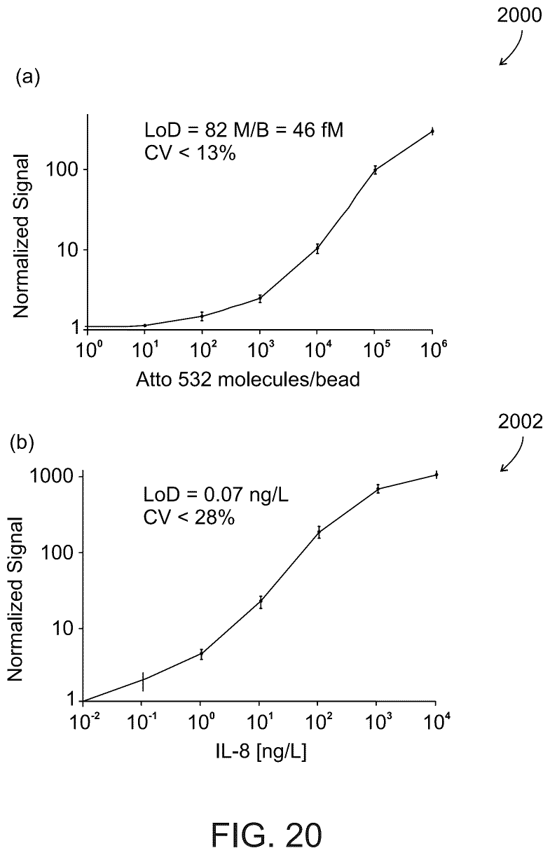

FIG. 20 shows a plot 2000 of the dose response of the system to fluorescently labeled magnetic beads with different numbers of fluorescent molecules per bead, and a plot 2002 of the dose response of an assay using human interleukin 8 (IL-8) as the target molecule. For plot 2000, different concentrations of biotinylated Atto 532 fluorescent dye (ATTO-TEC, AD 532-71) were attached to streptavidin-coupled M-280 superparamagnetic beads (ThermoFisher Sci. Waltham, MA, USA). Each sample consisted of approximately 100,000 beads mixed with biotinylated Atto 532 dye at concentrations of 0, 1, 10, 1×10², 1×10³, 1×10⁴, 1×10⁵, and 1×10⁶ molecules per bead. The reaction buffer solution contained phosphate buffered saline (Sigma-Aldrich, PBS), 10 mg/ml of bovine serum albumin (Sigma-Aldrich, BSA), and 0.01% of Tween-20 (Sigma-Aldrich, Tween-20). The beads and the fluorescent molecules were mixed and incubated for one hour at room temperature. Prior to the conjugation, the magnetic beads were photobleached for 18 hours in the buffer solution. Details of the photobleaching method are given in S. Roth, O. Hadass, M. Cohen, J. Verbarg, J. Wilsey, and A. Danielli, “Improving the Sensitivity of Fluorescence-Based Immunoassays by Photobleaching the Autofluorescence of Magnetic Beads,” Small, 1803751 (2019), DOI: 10.1002/smll.201970016. Following incubation, buffer replacement was made with a detection buffer solution (PBS×1, 10 mg/ml of BSA, 0.2% of Tween-20). Each sample was divided into four portions of 100 μL each. Three out of the four portions were loaded into 3 wells of a 96-well plate and measured using the high throughput OMB system (n=3). At the blank concentration (i.e., zero fluorescent molecules per bead), two samples of 400 μL each were prepared and six 100 μL each portions were measured (x=6). This experiment was repeated five times (n=5) on five different days and the mean of the average signal from each experiment was calculated. The limit of detection (LoD) was calculated as three standard errors over the blank measurement, and the dynamic range was determined by the ratio between the highest and lowest detectable concentrations. The limit of detection (LoD) for Atto 532 was calculated to be 82 fluorescent molecules per bead, which for a 300 μL sample volume, corresponds to a concentration of 46 fM, with a 4-log dynamic range. The coefficient of variance (CV) for all three measurements on each day was less than 23%. The day-to-day reproducibility for five repetitions of this experiment (n=5) on 5 different days, taking the mean of the three measurements on each day, was less than 13% over the entire dynamic range, as shown by the error bars of plot 2000.

The IL-8 assay shown in plot 2002 was carried out in a reaction buffer using three components: (1) a commercial IL-8 assay kit (BioRad, CXCL 171BK31MR2), containing biotinylated detection antibodies and magnetic beads conjugated to capture antibodies, (2) a Bio-Plex Pro Reagent Kit III (Bio-Rad, #171304090M), consisting of streptavidin phycocrythrin (SA-PE) fluorescent dye, and (3) a recombinant human IL-8 protein (#574202) that was obtained from BioLegend (San Diego, USA). To obtain a dose response measurement in buffer, 50 μL of reaction buffer containing magnetic beads (˜4000 beads/reaction) were mixed with 50 μL of increasing concentrations of IL-8 (Biolegend, San Diego, CA, Cat. #574202), resulting in final concentrations of 0, 0.01, 0.1, 1, 10, 1×10², 1×10³, and 1×10⁴ ng/L. The reaction mixtures were incubated for one hour at room temperature, followed by a 30-minute incubation with 50 μL of 1× biotinylated detection antibody solution and then a 20 min incubation with 80 μL of 1× streptavidin-phycocrythrin complex (SA-PE) solution. All incubation steps were performed in a 96 wells plate on a rotary shaker at room temperature. To remove the unbound SA-PE from the solution, a single buffer replacement was performed at the end of the protocol with detection buffer, using a MagJET magnetic separation rack (ThermoFisher Sci, Waltham, MA, USA, Cat. #MR03). For each concentration, three triplicates (n=3) were loaded into three wells on a 96-well plate and measured using the high throughput OMB system. At the blank concentration, six samples were prepared and measured (n=6).

For the IL-8 assay, the calculated LoD was 0.07 ng/L, and the coefficient of variation was less than 28% across the entire range. The signal saturates at 1000 ng/L, and therefore, the dynamic range is approximately 4-log.

FIG. 21 shows a plot 2100 of the results of a rapid E-gene-based RT-PCR assay for the detection of SARS-Cov-2 in RNA samples from COVID-19 patients, who had tested positive in a standard PCR test, and healthy subjects, who had tested negative is a standard PCR test, using the high throughput OMB system. The RT-PCR reactions for the OMB-based detection were performed using the TaKaRa One Step PrimeScript III RT-PCR kit (Cat. #RR600A, TaKaRa, Shiga, Japan) and MasterCycler ×50 PCR system (Eppendorf, Hamburg, Germany) with fast temperature ramping rate. The reaction mixture for each E-gene assay contained 10 μL of the ×2 One Step PrimeScript III RT-PCR mix, 0.8 μL (400 nM) of each primer (Forward and Reverse), 0.4 μL (200 nM) of the modified double-quenched probe, 5 μL of the sample (RNA extract), and 3 μL of PCR grade water for total reaction volume of 20 μL.

The reaction mixtures were subjected to five minutes at 55° C. (the reverse transcription step), followed by ten seconds at 95° ° C. (Reverse-Transcriptase inactivation and activation of the Taq polymerase step) and 40 PCR cycles of five seconds at 95° C., five seconds at 58° C., and five seconds at 60° C. Following the completion of the PCR stage of the process, the reaction products (20 μL) were transferred to a 96-well plate, pre-loaded with ˜25,000 streptavidin-coupled magnetic beads per well (M-280 Streptavidin, ThermoFisher Sci. Waltham, MA, USA) in 80 μL of PBST buffer (PBS×1, 0.05% of Tween-20), mixed by pipetting and incubated under constant shaking (RH-24 3D Gyratory Rocker, MIULAB, Hangzhou, China) for five minutes at room temperature. The final incubation volume was 100 μL. Subsequently, the beads were collected by setting the plate on the MagJET separation rack (ThermoFisher Sci. Waltham, MA, USA) for two minutes. The liquid was discarded, and the beads were resuspended in 100 μL of fresh PBST buffer.

A total of 76 clinical samples (35 positive and 41 negative), as well as a negative control sample, in which the RNA extract was substituted by PCR-grade water, and a positive control sample, which contained 10 copies of the SARS-COV-2 target in PCR-grade water, were loaded into 78 wells on a 96-well plate and measured using the high throughput OMB system.

The OMB assay successfully distinguished all the known positive subjects from the known negative subjects. For this test, the well plate was moved to sequentially read the fluorescent emission from the aggregated clump of beads in each well.

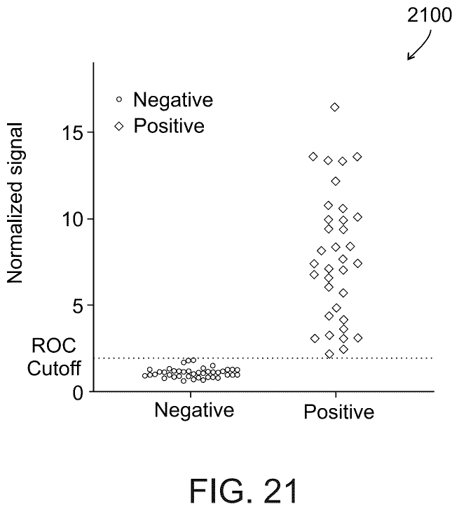

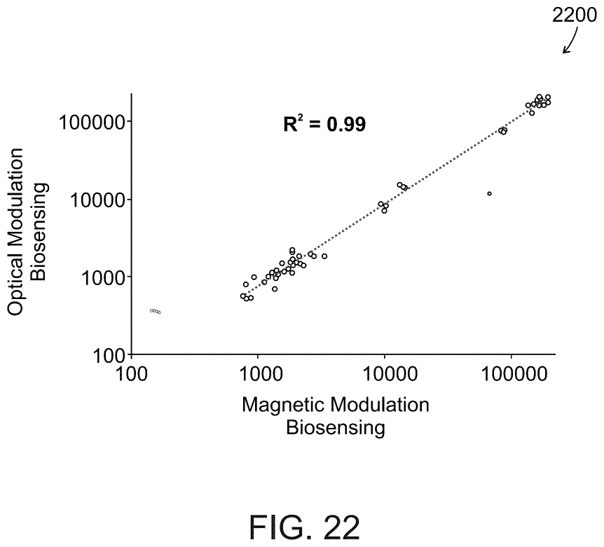

FIG. 22 shows a plot 2200 of the results of the test shown in plot 2000 for the dose response to fluorescently labeled magnetic beads with different numbers of fluorescent molecules per bead using the high throughput OMB system, and a similar test measuring the dose response to fluorescently labeled magnetic beads with different numbers of fluorescent molecules per bead, using a magnetically modulated biosensing (MMB) system, described in the references cited above in the Background section. The correlation of the measurements using the two systems is very high, with R²=0.99.

### Some Design Details for Another Optical Modulation Biosensing System

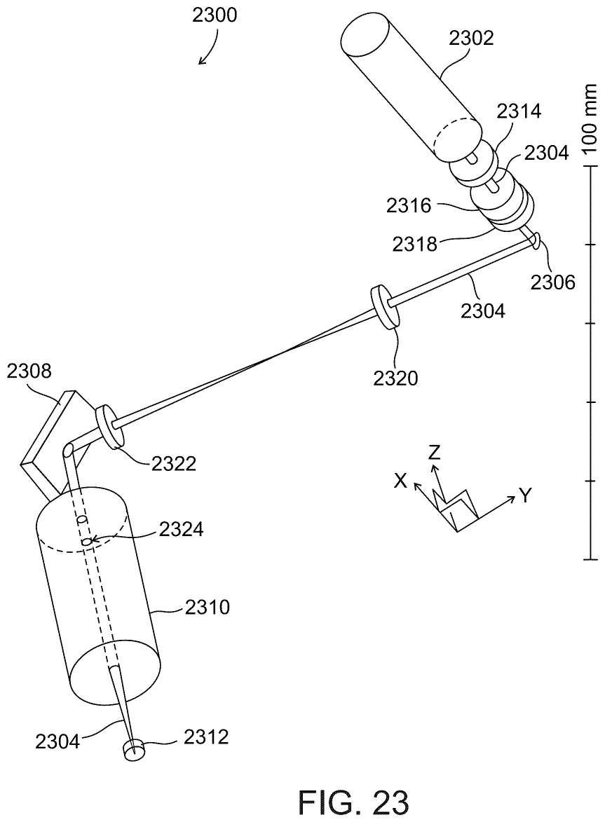

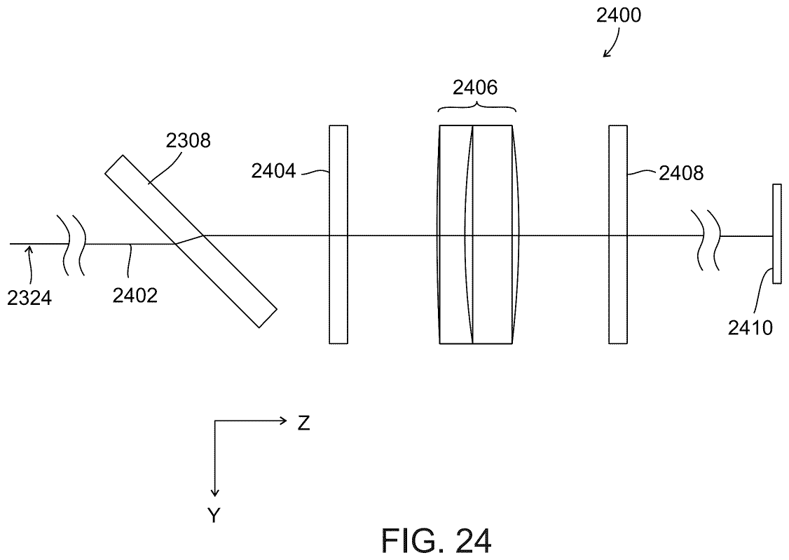

FIG. 23 shows a three-dimensional perspective view of another exemplary OMB system 2300, also similar to system 700 in FIG. 7. FIG. 23 shows only the optical elements involved in illuminating the magnetic beads with excitation light, while FIG. 24 shows some of the optical elements involved in measuring the fluorescent emission.

In FIG. 23, the z-axis is oriented vertically, pointing in an upward direction with respect to gravity, while the x and y axes are horizontal. A laser 2302 is oriented parallel to the x-axis, and emits an excitation light beam 2304 which initially propagates in the −x direction. A rotating mirror 2306, with its face oriented at a 45 degree angle in the x-y plane, reflects light beam 2304 from propagating in the −x direction to propagating in the −y direction. Light beam 2304 reflects from a dichroic mirror 2308, which has a face oriented at a 45 degree angle in the y-z plane, and after reflecting from dichroic mirror 2308, light beam 2304 propagates downward, in the −z direction. Light beam 2304 then passes through an objective lens 2310, which concentrates the beam into a small spot at the center of the bottom of a cuvette 2312, where the magnetic beads, not shown in FIG. 23, are aggregated into a clump by a permanent magnet, also not shown in FIG. 23, that is located just below cuvette 2312.

FIG. 23 shows some additional optical elements that are not shown in the schematic illustration in FIG. 7. After light beam 2304 exits laser 2302, it optionally passes through a neutral density filter 2314, which reduces its intensity. Reducing the intensity of laser beam 2304 may be useful to prevent or reduce photobleaching of the fluorescent reporter molecules in the magnetic beads, although it is potentially advantageous not to reduce the intensity of laser beam 2304 too much, since that will generally reduce the fluorescent emission light from the magnetic beads, which may reduce the signal to noise ratio of the fluorescent emission measurement. In system 2300, laser 2302 is optionally a GLM-001D-V2 green dot laser module, sold by Elite Optoelectronics Co., Ltd., in Xi′an, China, which produces a maximum output of 1 mW of green light at 532 nm, with a 20 raw beam diameter of 1.114 mm. Neutral density filter 2304 optionally reduces the intensity of the beam by a factor of 4, so the laser power that reaches the bottom of cuvette 2312 is 0.25 mW.

After passing through neutral density filter 2306, laser beam 2304 passes through lenses 2316 and 2318, which are used to adjust the spot size of beam 2304 at the bottom of cuvette 2312. Lens 2316 has focal length −25 mm, and diameter 12.7 mm, Thorlabs part no. LC1054-A, and lens 2318 has focal length +25 mm and diameter 12.7 mm, Thorlabs part no. LA1560-A. Lens 2318 is optionally kept at a distance of 29.5 mm from the laser, and lens 2316 can be varied in position between 2.05 mm and 8.72 mm from lens 2318, to control the spot size. For example, when lens 2316 is 2.87 mm from lens 2318, then the spot size, which is the diameter of the beam at the bottom of the cuvette, is 100 μm, if the lenses are 4.25 mm apart then the spot size is 150 μm, and if the lenses are 5.63 mm apart, then the spot size is 200 μm. If the spot size is too small, then only a small fraction of the clump of beads may be illuminated, and those beads may be significantly photobleached or saturated by the high intensity of the light, reducing the fluorescent emission signal. Small spot size can also increase the noise level of the fluorescent emission, due to statistical fluctuations in the number of fluorescent reporter molecules present within the spot, especially near the limit of sensitivity of the assay. If the spot size is too large, then much of the light beam may miss the clump of beads, reducing the fluorescent emission signal, without decreasing the background signal.

In order to aim laser beam 2304 to a position 0.8 mm from the center of the bottom of cuvette 2312, which will be to the side of the aggregated clump of beads, rather than at the center of the bottom of the cuvette where the clump is located, rotating mirror 2306 rotates by 1.145 degrees from its 45 degree orientation, using a single-axis galvanometer with a position sensor, model 6200H from Cambridge Technology. Rotating mirror 2306 has a clear aperture of 5 mm. In order to keep laser beam 2304 telecentric even when it is deflected to the side, a pair of relay lenses 2320 and 2322 are optionally used, to relay the pupil plane from rotating mirror 2306 onto an entrance pupil 2324 of objective lens 2310. This ensures that laser beam 2304 is centered at entrance pupil 2324 even when it is deflected to the side, and keeps it directed in the z-direction, but displaced by 0.8 mm to the side, when it exits objective lens 2310. Relay lenses 2320 and 2322 are both Thorlabs part no. LA1304-A, plano-convex lenses with focal length +40 mm, and diameter 12.7 mm. Lens 2320 is positioned so that the center of its right surface, which is its convex surface, is 40 mm from the center of rotating mirror 2306, and the center of the left surface of lens 2320, which is its planar surface, is 75.32 mm from the center of the right surface of lens 2322, which is its planar surface. The left surface of lens 2322, which is its convex surface, is 10 mm from the center of dichroic mirror 2308, and the center of dichroic mirror 2308 is 30 mm from entrance pupil 2324 of objective lens 2310. At these positions, lenses 2320 and 2322 produce a 1:1 real image of the pupil plane of rotating mirror 2306, at entrance pupil 2324 of the objective lens, which ensures that laser beam 2304 will be telecentric if objective lens 2310 is telecentric.

The dichroic mirror is optionally model Di02-R532-t3-25×36 from Semrock, cut down to a height of 18 mm in the x-direction and a width of 24 mm in the y-z plane. The objective lens is optionally a Mitutoyo long working distance objective ×10 M PLAN APO 10x, part number MY10X-803 from Thorlabs, or part number 46-144 from Edmund Optics. For this objective lens, the entrance pupil 2324 is 7.6 mm below the top of the objective lens. The objective lens is cylindrical, 61 mm long, and 32.2 mm in diameter. The focal plane of this objective lens is located 87.4 mm below the entrance pupil, which is 95 mm below the top of the objective lens, and 34 mm (the “working distance” of the objective lens) below the bottom of the objective lens. Objective lens 2310 may be modeled, for ray-tracing calculations, as a single thin lens of focal length 20 mm, located 20 mm above its focal plane, which is below the physical lens. Cuvette 2312 is optionally positioned so that the inside bottom surface of the cuvette is located at the focal plane of the objective lens. Optionally, the position of cuvette 2312 is adjusted to make the focal plane of objective lens 2310 coincide with the inside bottom surface of the cuvette, taking into account that the cuvette has a solution in it, for example a buffer solution that is largely water, and is assumed to have the refractive index of water, and assuming, for example, that the solution is 50 micro-liters in volume, which would make the solution 1.76 mm deep for a cuvette that is a circular cylinder of diameter 6 mm. Alternatively, depending on the nature and depth of the solution in the cuvette, different values are used for the index of refraction of the solution and/or its depth, in positioning the cuvette so that the focal plane coincides with the inside bottom surface of the cuvette. Even though the focal plane of objective lens 2310 is at the bottom of the cuvette, laser beam 2304 is optionally focused a few millimeters above the bottom surface of the cuvette, by adjusting the distance between lenses 2316 and 2318 as explained above, so that laser beam 2304 makes a larger spot at the bottom of the cuvette, where it illuminates the clump of beads, than if laser beam 2304 were focused exactly there.

FIG. 24 shows a side view 2400 of some optical components used to collect and measure the fluorescent emission from the clump of magnetic beads at the bottom of the cuvette. In FIG. 24, the z-axis, which is vertical with respect to gravity, goes to the right, and the plane of the drawing is the y-z plane. Central fluorescent emission light ray 2402, coming straight up from the center of the bottom of the cuvette, follows the same path as the center of laser beam 2304, including going through entrance pupil 2324 of the objective lens, until it reaches dichroic mirror 2308. Central light ray 2402, and all the other fluorescent emission light rays, go through dichroic mirror 2308, rather than reflecting from it as laser beam 2304 does, because the fluorescent emission light rays have longer wavelengths than the laser beam, ranging from 550 nm to 590 nm, while the laser beam has a wavelength of 532 nm. Central ray 2402, as well as the other fluorescent emission light rays, is displaced in the y-direction by 1 mm when it passes through dichroic mirror 2308. The fluorescent emission light rays, including ray 2402, then pass through a narrow-band filter 2404, a tube lens 2406, and another narrow band filter 2408. The fluorescent emission light rays form an image of the bottom of the cuvette at sensor array 2410.

As noted previously, the center of dichroic mirror 2308 is 30 mm from entrance pupil 2324 of objective lens 2310. Narrow-band filters 2404 and 2408 are both optionally Semrock FF01-560/25-25 filters, cut down to a diameter of 24 mm, which largely admit light of the range between 550 nm and 590 nm expected for the fluorescent emission, but largely block light of other wavelengths, and in particular they largely block light of 532 nm, including any stray light from laser beam 2304. Specifically, each filter transmits at least 93% of the light within the 25 nm wide transmission band from 548.5 nm to 572.5 nm, and has an optical depth of at least 5 between 200 nm and 531 nm, an optical depth of at least 10 between 589.5 nm and 623.5 nm, and an optical depth of at least 5 between 623.5 nm and 925 nm. Filters 2404 and 2408 optionally are respectively positioned, for example, 10 mm to the left and 10 mm to the right of lens 2506, as seen in FIG. 24. The left side of the center of filter 2408 is optionally located 13.883 mm from the location on the right side of dichroic mirror 2308 (as seen in FIG. 24) where center light ray 2402 emerges after passing through the dichroic mirror. The filter itself, not including its mounting, is 2 mm thick, and the dichroic mirror, not including its mounting, is 3 mm thick.

Tube lens 2406 is optionally an Edmund Optics convex achromatic lens, VIS 0 coated, 25 mm in diameter, and with focal length 175 mm, Edmund Optics drawing number 47644. The lens is 9 mm thick at its center where central light ray 2402 crosses it, and a light sensor array 2410 is located at a nominal distance of 173.8 mm to the right (in FIG. 24) of the center of the right surface of lens 2406, which is 241.92 mm from entrance pupil 2324 of objective lens 2310. This distance is optionally adjustable by +5 mm, to project a well-focused image of the bottom of cuvette 2312 on the sensor array. Sensor array 2410 is optionally a SONY IMX174 sensor, mounted in a FLIR Grasshopper3 2.3 MP Mono USB3 Vision—C mount camera. This sensor array has 1920×1200 pixels, each pixel 5.86 μm across, with an effective area of 11.2512 mm×7.032 mm, and a diagonal of 13.27 mm. With the objective lens and tube lens configuration described, the image on the sensor has a magnification of 8.72, and the resolution on the bottom of the cuvette is about 1 to 1.5 μm, with a field of view of 1.290 mm×0.806 mm. Optionally, the camera is oriented so that the wide direction (1.290 mm) of the field of view corresponds to the direction along which the illumination spot moves on the bottom of the cuvette, when rotating mirror 2306 rotates, which will make it possible to include both positions of the spot in the same image, similar to what is shown in FIG. 8, although that image was made using a different configuration of optical elements.

### Measuring Emission as a Function of Time

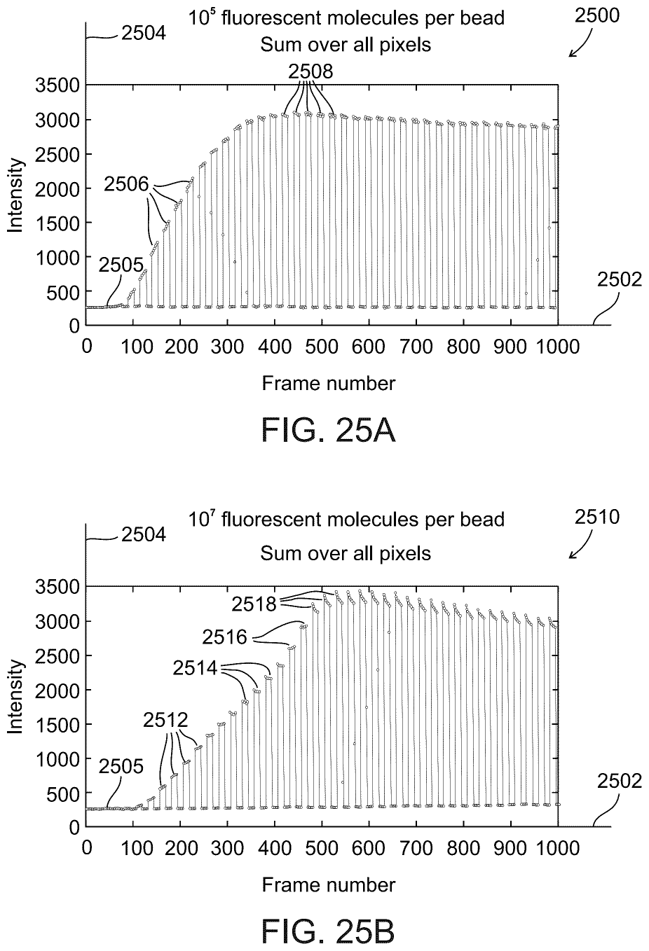

FIGS. 25A and 25B compare the measured fluorescent emission as a function of time, starting before the beads begin to aggregate and continuing after the beads have completed aggregating into a clump, for two different concentrations of target molecules in the sample. FIG. 25A shows a plot 2500, with a horizontal axis 2502 representing time, in terms of the frame number, and vertical axis 2504 representing the intensity of the fluorescent emission, in Mean Gray Level (normalized to the emission from the autofluorescence of the beads, as in FIG. 9 and FIG. 20) averaged over all the pixels in the image, which covers the location in the container in which the clump forms. The successive frames are recorded at intervals of 20 milliseconds, so the full range of the plot, from frame 0 to frame 1000, represents a time period of 20 seconds. Plot 2500 shows the case where the concentration of target molecules in the sample produces 10⁵ fluorescent reporter molecules adhering to each bead. The assay was the same kind of assay described for FIG. 20.

Region 2505 of the plot shows the emission before the beads start to aggregate, when the recorded intensity just represents the dark response of the light detector. The location in the container is alternately illuminated by the excitation light for 0.25 seconds, and not illuminated for 0.25 seconds. When the location is not illuminated, the measured intensity is at the level of the dark response of the detector. The beads begin to aggregate at about frame 90, and during periods 2506 when the excitation light is illuminating the area, the measured intensity starts to increase approximately linearly with time, as more and more beads aggregate in the location. Although the fluorescent emission intensity of each bead decreases slightly within each illumination time interval due to photobleaching, the number of beads at the location is still increasing rapidly enough at this time that the loss of intensity due to photobleaching is negligible, and the measured intensity increases nearly linearly with time. At later times, as the aggregation of the beads into a clump starts to become complete, the measured intensity of fluorescent emission, during the time intervals when the location is illuminated with excitation light, starts to increase more slowly, and when the clump is fully aggregated, the measured intensity of fluorescent emission becomes almost independent of time. Looking at the curve during time intervals 2508, when the aggregation of the beads is complete, one can see the intensity decreasing slightly during each time interval when the beads are being illuminated by excitation light, due to photobleaching of the fluorescent reporter molecules by the excitation light. Because the clump is no longer changing in size, the photobleaching rate of the beads can be determined by measuring the decrease in fluorescent emission within each interval 2508. But between those time intervals, when the excitation light is not illuminating the beads, the fluorescent reporter molecules largely recover from the photobleaching. During each of these dark intervals, the emission coefficient of the beads, defined as the ratio of emission power of a bead to the power of the excitation light received by it, returns almost to the value it had at the beginning of the previous illumination interval, before the photobleaching of that interval. However, the emission coefficient appears not to recover completely from the photobleaching after each dark interval, and possibly as a result of this, there is a gradual decrease in the measured fluorescent emission intensity over time, perhaps due to the net difference between the photobleaching and the recovery.

As may be seen from plot 2000 in FIG. 20, at relatively low levels of target molecule concentration, corresponding to between 10³ and 10⁵ fluorescent reporter molecules per bead, the fluorescent emission per bead is nearly a linear function of the number of fluorescent reporter molecules. At 106 molecules per bead, the fluroscent emission starts to saturate, being only about 3 times greater than at 105 molecules per bead, and at 107 molecules per bead, not shown in FIG. 20, the fluorescent emission per bead goes down again, to a level about equal to the fluorescent emission at 105 molecules per bead, when the beads are fully aggregated. The inventors believe that this saturation and downturn in fluorescent emission, at a high concentration of fluorescent molecules per bead, may be due to the beads being photobleached by their own fluorescent emission, and the fluorescent emission of other nearby beads. In this regime of high concentration of target molecules, the fluorescent emission intensity, when the beads are fully aggregated, does not provide an unambiguous measure of the number of fluorescent reporter molecules per bead, or of the concentration of target molecules in the sample. However, the inventors have found that measuring the fluorescent emission as a function of time, while the beads are aggregating, and measuring the rate of photobleaching of the beads, may provide information on the number of fluorescent reporter molecules per bead, and hence on the concentration of target molecules in the sample, in this regime, as illustrated in FIG. 25B. Using one or both of these effects may make it possible to increase the dynamic range of the assay by an order of magnitude.

FIG. 25B shows a plot 2510 of the fluorescent emission as a function of time, for the case of 107 fluorescent reporter molecules per bead. A horizontal time axis 2502 shows the frame number, as in plot 2500, with the same timing of successive frames, and a vertical axis 2504 shows the fluorescent emission using the same units as plot 2500. As in plot 2500, the excitation light illuminates the beads for an interval of 0.25 seconds, followed by no illumination of the beads by excitation light for 0.25 seconds, and repeating the cycle. The measured fluorescent emission is summed over all pixels of the image, as in plot 2500, with the image covering the location on the bottom of the container where the beads will aggregate.

In region 2505 of plot 2510, showing the measurements before the beads have started to aggregate, there are no beads or almost no beads in the image, and the measured emission is at the dark level of the sensor, as in plot 2500. The beads start to aggregate at about frame 100, and initially, at time intervals 2512, the fluorescent emission increases approximately linearly with time, though somewhat more slowly than in plot 2500. At this time, the photobleaching during each time interval when the excitation light is illuminating the beads decreases the fluorescent emission more slowly than it is increasing due to the aggregation of the beads, and fluorescent emission goes up during each of time interval 2512. As the beads continue to aggregate, during time intervals 2514, the rate of increase in emission due to the aggregation becomes less than the rate of decrease in emission due to the photobleaching, during each interval when the beads are illuminated by the excitation light, and the emission decreases during each of these intervals, though the beads largely recover from the photobleaching during the intervals when the excitation light is not illuminating the beads, and the fluorescent emission at the beginning of each interval when the excitation light illuminates the beads is greater than it was in the previous such interval, due to the continued aggregation of the beads. But the aggregation is still proceeding more slowly than at the same level of emission in plot 2500.

In intervals 2516, starting when the emission of the clump is about 80% of its peak value, the rate of aggregation apparently increases, and the fluorescent emission starts to increase more quickly. Also during intervals 2516, the increase in fluorescent emission due to the aggregation is apparently greater than the decrease due to photobleaching, and during each of intervals 2516 the fluorescent emission increases with time. The time required for the fluorescent emission to grow from 80% to 100% of its peak value is only about 60 frames (1.2 seconds) in plot 2510, while in plot 2500 this time is longer, about 100 frames (2 seconds).

Finally, in time intervals 2518, fluorescent emission stops growing. The clump may be fully aggregated at this time, or, if there is any further aggregation still taking place, the increase in the number of beads may be balanced by a decrease in emission per bead, perhaps due to self-photobleaching, so that the total emission does not increase. As in plot 2500, in plot 2510 the fluorescent emission coefficient of the beads does not quite fully recover during each dark interval, and the fluorescent emission slowly decreases over time after the clump is fully aggregated. In this regime, as in intervals 2508 in plot 2500, the photobleaching rate can be determined by measuring the rate of decrease in fluorescent emission during the intervals when the beads are illuminated by the excitation light. In plot 2500, the fluorescent emission decreases by about 1.7%, due to photobleaching, during each interval 2508. In plot 2510, the photobleaching rate is much greater, and the fluorescent emission decreases by about 7%, due to photobleaching, during each interval 2518. The greater photobleaching rate in plot 2510 may be due to a contribution of the emission light to photobleaching, in addition to the photobleaching from the excitation light, or to some other collective effect of neighboring fluorescent molecules on the emission rate of each fluorescent molecule.

Even though the peak fluorescent emission intensity, when the clump is fully aggregated, is nearly the same in plot 2500 and plot 2510, and this quantity could not be used to reliably distinguish the case of 10⁵ fluorescent reporter molecules per bead from the case of 10⁷ fluorescent reporter molecules per bead, two other features of the measured fluorescent emission as a function of time are very different for the two cases. The time for the emission signal to grow from 80% to 100% of its peak value is shorter, by about a factor of 2, for 10⁷ fluorescent molecules per bead than for 10⁵ fluorescent molecules per bead. And the photobleaching rate is about 4 times greater for 10⁷ fluorescent molecules per bead than for 10⁵ fluorescent molecules per bead. Either one of these features, or both of them in combination, probably could be used to reliably distinguish the case of 10⁷ fluorescent molecules per bead than from any case with significantly fewer fluorescent molecules per bead, such as 10⁶ or 10⁵ fluorescent molecules per bead. Even if the peak fluorescent emission is not used to measure fluorescent molecules per bead beyond about 10⁶ molecules per bead, in some embodiments of the invention these two features are used to extend the dynamic range of such an assay from a maximum of 10⁶ fluorescent molecules per bead by at least another order of magnitude, up to a maximum of at least 10⁷ fluorescent molecules per bead.

In order to use these features of the fluorescent emission as a function of time, for any similar bioassay, these features could be calibrated by performing a series of assays with different numbers of fluorescent molecules per bead, and seeing how the number of fluorescent molecules per bead affects the time it takes for the emission to increase from 80% to 100% of its peak value, and how the number of fluorescent molecules per bead affects the photobleaching rate when the clump is fully aggregated and no longer changing over time. Although the details of how the number of fluorescent molecules per bead affects these features probably varies depending on the number of beads in the clump, the type of beads used, the type of fluorescent reporter molecules used, the brightness of the excitation light, and other parameters, once the calibration is done for an assay with a given set of the parameters, these two features, together with the peak fluorescent emission, are optionally used to determine the number of fluorescent molecules per bead, and hence the concentration of target molecules in the sample. By secing how each of these three features varies with the number of fluorescent molecules per bead, and by seeing the noise level in each of the features over the dynamic range that the assay is expected to cover, the calibration can also reveal which combination of the features, with which relative weights, should be used to maximize the sensitivity of the assay over its entire dynamic range. For example, for the assay shown in FIGS. 25A and 25B, such a calibration would probably show that most weight should be given to the peak fluorescent emission for up to 10⁵ fluorescent molecules per bead, while for 10⁶ or 10⁷ fluorescent molecules per bead much more weight should be given to the time for the emission to grow from 80% to 100% of its peak value, and especially to the photobleaching rate.

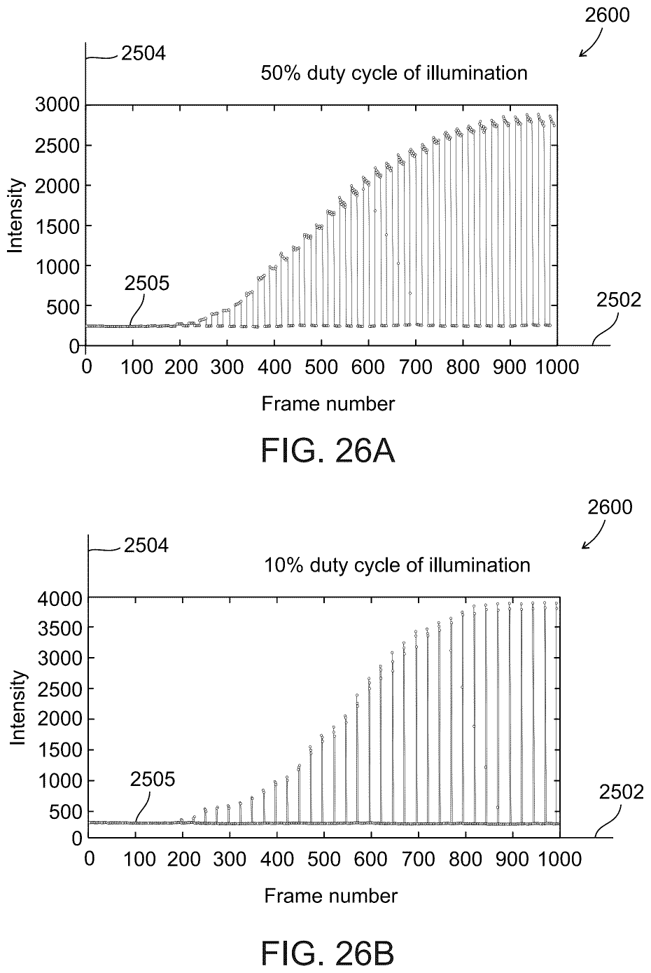

FIGS. 26A and 26B show the emission as a function of time during and after the aggregation of the clump, for two cases with different duty cycles for the illumination of the beads. These plots show that when the beads are illuminated for a smaller fraction of the cycle of illuminating and not illuminating the beads, the emission is less affected by photobleaching, because there is less photobleaching and more time for the beads to recover from photobleaching. FIG. 26A shows a plot 2600 of the intensity of the emission signal, summed over all pixels in the field of view, as a function of time, with an illumination duty cycle of 50%, and FIG. 26B shows a plot 2602 of the intensity of the emission signal as a function of time, with an illumination duty cycle of 10%. For both plots, time is shown as frame number on a horizontal axis 2502, and intensity is shown in the same normalized Mean Gray Value units, on a vertical axis 2504, as in FIGS. 25A and 25B. There were 10⁵ fluorescent molecules per bead, as in FIG. 25A. The frame rate is 50 frames per second, or 0.02 seconds per frame, so the horizontal axis, which goes from frame 0 to frame 1000, covers 20 seconds. Each illumination cycle, with the excitation light first illuminating the field of view and then not illuminating the field of view, is 25 frames, or 0.5 seconds. Also as in FIGS. 25A and 25B, the field of view covers the area over which the clump of beads will be located, once it is fully aggregated. Initially, in region 2505 of the plot, there are no beads aggregated in the field of view, and the signal is equal to the dark level of the sensor signal. Once the beads begin to aggregate, at about frame 200, the signal starts to rise, and reaches its maximum value when the beads are fully aggregated into a clump, at about frame 850. In plot 2600, the peak intensity, when the beads are fully aggregated, is about 2900. In plot 2602, which differs from plot 2600 only in that the beads are illuminated by the excitation light only 10% of the time, instead of 50% of the time as in plot 2600, the peak intensity is about 3900. This shows that the photobleaching at 50% duty cycle reduces the emission by at least 25% in steady state when the beads are fully aggregated. Another test, using a 20% duty cycle, not shown in FIGS. 26A and 26B, shows that the peak intensity is also about 3900, nearly the same as with a 10% duty cycle, which suggests that at a 10% duty cycle, photobleaching reduces the peak intensity very little, in steady state when the beads are fully aggregated. It can be seen in plot 2602, that when the duty cycle is 10%, photobleaching reduces the emission by about 3% within the short time, 0.05 seconds, that the beads are illuminated, while in plot 2600, photobleaching reduces the emission by about 5% during the 0.25 seconds that the beads are illuminated. Photobleaching apparently occurs more quickly during the first 0.05 second of illumination, and then slows down, which can also be seen directly in plot 2600. But the emission coefficient apparently largely recovers from photobleaching during the 0.45 seconds dark period in plot 2600, so the net effect of photobleaching on the peak steady state emission is very small in plot 2602, as indicated by the fact that the peak emission hardly changes when the duty cycle is increased from 10% to 20%. These results suggest that, at least for the illumination level and 0.5 second cycle time used in these tests, accurate measures of fluorescent emission, hardly affected by photobleaching, can be obtained by illuminating the beads with a duty cycle of 20% or less.

Preliminary data obtained by the inventors, not shown in FIGS. 26A and 26B, show that, with an illumination duty cycle of 10% to 20%, the rate of increase of the emission signal, when it is between 30% and 70% of its peak value, correlates well with the number of fluorescent molecules per bead, even in the range between 105 and 107 fluorescent molecules per bead, where the peak emission signal does not provide a good indication of the number of fluorescent molecules per bead.

In some embodiments of the invention, instead of determining the peak emission level of the beads by measuring the emission level as a function of time until it stops rising, or until it starts to decrease slowly, the emission level is measured as a function of time only during the time that it is still rising, before the beads have completely aggregated, and the peak emission level is estimated from the rate of rise of the emission level. This may be done fairly accurately, because, especially at relatively low numbers of fluorescent molecules per bead, the shape of the envelope of the emission level as a function of time, for a given magnet configuration and a given number and type of magnetic beads, a given shape and size of the container, and a given type and level of solution in the container, is typically almost independent of the number of fluorescent molecules per bead. If a series of assays is being done, in which all of these factors remain constant, and only the number of fluorescent molecules per bead varies, then it may be possible to accurately estimaate the peak level of emission from the initial rate of rise, once that relationship has been calibrated.

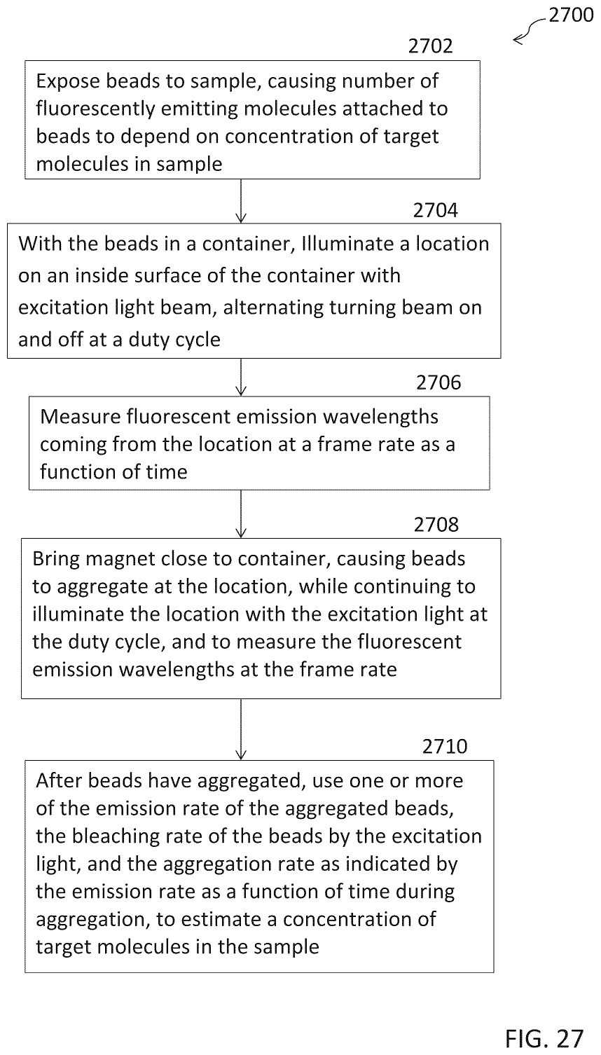

FIG. 27 shows a flowchart 2700, for a bioassay method using magnetic beads with attached reporter molecules, for example fluorescent reporter molecules. At 2702, the beads are exposed to a sample that is being assayed, causing the number of reporter molecules attached to the beads to depend on the concentration of target molecules in the sample. This can be accomplished, for example, by using a sandwich assay, or a FRET assay, or any known type of bioassay using beads with attached reporter molecules that produce optical emission, for example as described above for FIG. 1, or as described above in the section “High throughput optical modulation biosensing system.”

At 2704, with the prepared beads in a container, for example surrounded by a solution held in the container, a location on an inside surface of the container, for example the bottom of the container, is illuminated with an excitation light beam, with a repeated cycle where the beam alternates between illuminating the location and not illuminating the location. The location that is illuminated is the location where the beads will aggregate to form a clump, but the excitation light optionally starts to illuminate this location on and off even before the beads begin to aggregate there. At 2706, also before the beads begin to aggregate, any light coming from the location, in a wavelength range of the fluorescent emission that the beads will produce, is measured as a function of time. For example, the wavelength range is optionally between 550 nm and 590 nm, for fluorescent emission excited by excitation light at 532 nm. Initially, with no beads or almost no beads at that location, there will be little or no light coming from the location at those wavelengths.

At 2708, aggregation of the beads into a clump is initiated, for example by bringing one or more magnets close to the outside of the container adjacent to the location, producing a magnetic field at the location that will cause the beads to aggregate there. Alternatively, the one or more magnets are already located outside the container, adjacent to the location and producing a magnetic field there, and the beads are first introduced into the container at this time, causing the beads to start aggregating at the location. During the aggregation process, the excitation light continues to illuminate the beads on and off, and the intensity of light at the fluorescent emission wavelengths, coming from the location, continues to be measured as a function of time. The location now has a growing clump of beads producing fluorescent emission, which is being measured as a function of time. Optionally, an integrated level of fluorescent emission from the location is measured, covering the area of the fully aggregated clump but not extending very far beyond that area. Alternatively, measuring the fluorescent emission is done using a camera, such as a CCD camera, that produces a set of successive images of the location, at a frame rate over time, in the wavelength range of the fluorescent emission, covering the area of the fully aggregated clump. Producing images of the distribution of fluorescent emission, rather than only measuring an integrated level of the fluorescent emission from the whole area of the location, has the potential advantage that it can provide information about the shape of the growing clump of beads, rather than only about its integrated emission which may depend mostly on its total area on the inner surface of the container, and can show the process by which the beads are attracted toward the growing clump over time.

At 2710, after the beads have fully aggregated, information about the fluorescent emission rate from the clump is used to estimate a concentration of the target molecules in the sample. The information used can include, for example, the peak emission rate from the clump, when it is fully aggregated; the growth rate of the emission; and the photobleaching rate of the beads in the clump. As described for FIGS. 25A and 25B, the total emission rate from the clump can provide a good estimate of the number of fluorescent reporter molecules attached to the beads, but only up to a certain number of attached fluorescent reporter molecules per bead, and beyond that level the total emission saturates, or even decreases, with increasing attached fluorescent reporting molecules, and does not provide a good measure of the number of attached fluorescent reporter molecules. At higher numbers of attached fluorescent reporting molecules, the time required for the emission to grow, for example from 80% to 100% of its peak value, or from 30% to 70% of its peak value, can provide a good measure of the number of attached fluorescent reporter molecules per bead, as can the photobleaching rate.

Trapping Magnetic Beads in a Channel as Fluid Flows Past them

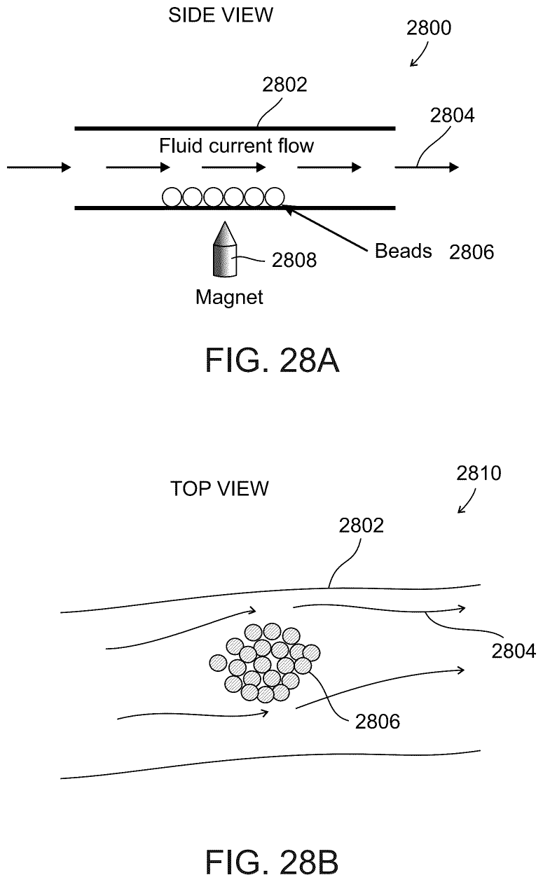

FIGS. 28A and 28B show a channel used in an assay for a concentration or presence of target molecules in a biological sample, based on optical emission, such as fluorescent emission, from magnetic beads. Optionally, the channel is a microfluidic channel, for example a microfluidic channel found in a microfluidic cartridge used for the assay, such as the microfluidic cartridge shown in FIG. 18. The channel is, for example, etched in a solid block of material, such as plastic or glass, or the channel is made from tubing. FIG. 28A shows a side view 2800 of the channel. A fluid 2804 flows along the channel past magnetic beads 2806, that are adhering to the bottom surface of the channel because of a magnetic field produced by a magnet 2808 located just beneath the channel. The flow of fluid 2804 past the beads washes away from the vicinity of the beads loose fluorescent reporter molecules that would otherwise produce a background level of fluorescent emission appearing to come from the beads, even if there were no fluorescent reporter molecules attached to the beads, because there were no target molecules in the sample. With the loose fluorescent reporter molecules washed away, a more accurate measure may be made of fluorescent emission from the beads, even without measuring and correcting for a background emission level. FIG. 28B shows a top view 2810 of channel 2802, with fluid 2804 flowing past magnetic beads 2806, which form a clump, optionally only one layer thick, on the bottom of the channel.

Alternatively, instead of the one or more magnets being located below the bottom of the channel, and the beads aggregating on the bottom surface of the channel, the one or more magnets are located next to a side surface or even a top surface of the channel, and the beads aggregate on the side surface or top surface, or two or more magnets are located adjacent to different surfaces of the channel, and the beads aggregate on different surfaces of the channel. However, the inventors have found that the beads generally aggregate more quickly and are trapped more effectively if the magnet is located beneath the channel, and beads aggregate on the bottom surface of the channel.

Optionally, the surface of the channel where the beads aggregate is rough on a distance scale of the bead diameter, and/or has one or more depressions comparable in size to the expected aggregation, which help to protect the trapped beads from being swept away by the flow of the fluid.

Optionally, there is a pumping mechanism, not shown in FIG. 28, that controls the flow speed of the fluid, and suspended beads, in channel 2802. For example, the beads and fluid are stored in a reservoir located at one end of the channel, and the pumping mechanism creates a pressure difference between the reservoir, and the other end of the channel. The pumping mechanism, for example, can comprise a piston that presses on the reservoir, creating a higher pressure in the reservoir, and/or a piston that draws fluid from a chamber at the other end of the channel, creating a lower pressure there. By controlling the pressure difference along the channel, the pumping mechanism controls the flow speed of the fluid along the channel, adjusting it to a value that will allow the beads to be trapped by the magnetic field in the channel. Alternatively, other mechanisms are used to drive the flow and optionally to control the flow speed, for example gravity, capillary flow, ion or magnetic based flow, or ultrasound.

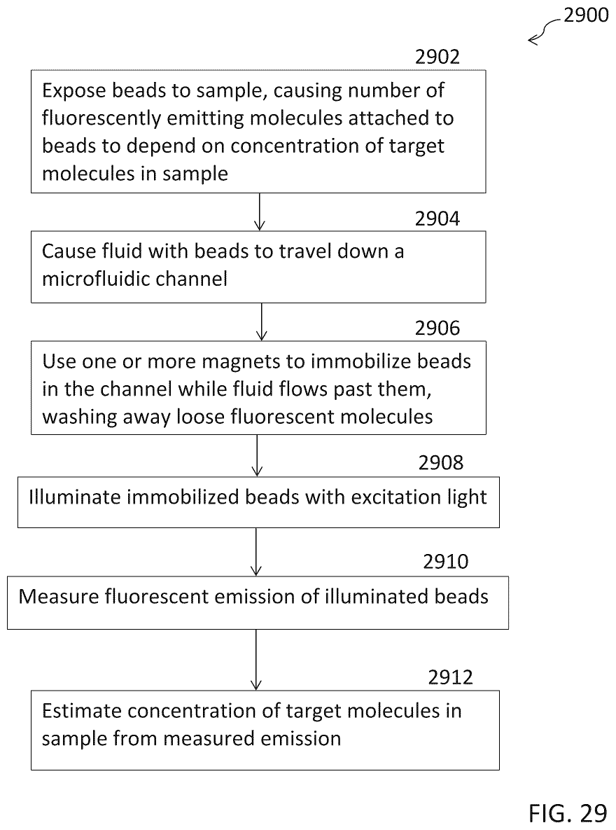

FIG. 29 shows a flowchart 2900 for a method of performing a bioassay using a channel such as channel 2802 in FIGS. 28A and 28B, for example a microfluidics channel. At 2902, the beads are exposed to a sample that is being assayed, causing the number of reporter molecules attached to the beads to depend on the concentration of target molecules in the sample. This can be accomplished, for example, by using a sandwich assay, or a FRET assay, or any known type of bioassay using beads with attached reporter molecules that produce optical emission. The type and number of beads used, and the method of preparing them, is optionally like the method described above for FIG. 1, or described above in the section “High throughput optical modulation biosensing system,” for example.

At 2904, a fluid, with the beads immersed in it, is caused to flow along the channel, for example by applying a pressure difference between two chambers on opposite ends of the channel. For example, the channel is 4 mm wide, and 0.14 mm deep. Alternatively, the channel is less than 1 mm wide, or between 1 and 2 mm wide, or between 2 and 4 mm wide, or between 4 and 10 mm wide, or more than 10 mm wide, and the channel is less than 0.05 mm deep, or between 0.05 and 0.1 mm deep, or between 0.1 and 0.2 mm deep, or between 0.2 and 0.5 mm deep, or between 0.5 and 1 mm deep, or between 1 and 2 mm deep, or between 2 and 5 mm deep, or more than 5 mm deep. The fluid is optionally an aqueous solution, for example a buffer solution, for example as described above in the section “High throughput optical modulation biosensing system,” or any of the fluids described for system 100 in FIG. 1. The flow rate in the channel is optionally less than or equal to 1.65 microliters per second, or between 0.01 and 0.1 microliters per second, or between 0.1 and 1 microliters per second, or between 1 and 10 microliters per second, or between 10 and 100 microliters per second. At 2906, one or more magnets, located just beneath the floor of the channel, are used to trap at least some of the beads on the bottom surface of the channel, immobilizing them while the fluid flows past them. For example, the floor of the channel is optionally 1.65 mm thick, and a magnet, optionally with the same design described in FIG. 4, or with the design described above in the section “High throughput optical modulation biosensing system,” is optionally located with its tip just touching the outside of the bottom of the channel, so that the tip of the magnet is 1.65 mm below the bottom inner surface of the channel. Alternatively, the floor of the channel is a different thickness, and the tip of the magnet, whether it is touching the outside of the bottom of the channel or not, is a different distance from the bottom inner surface of the channel, for example less than 1 mm away, or between 1 mm and 2 mm away, or between 2 mm and 5 mm away, or more than 5 mm away. The flow speed is optionally low enough so that at least some of the beads, for example at least 50% of them, or at least 20% of them, or at least 10% of them, or at least 5% of them, or at least 2% of them, or at least 1% of them, or less than 1% of them, or at least 50 beads, or at least 20 beads, or at least 10 beads, are trapped on the bottom surface of the channel by the magnetic field of the magnet, as they flow past the magnet. Optionally, there are few enough trapped beads so that they form only a single layer on the bottom of the channel.

Optionally, before the assay is done, a series of calibration tests is done, using the same channel, magnet configuration and type and number of beads as will be used in the assay, with different flow speeds, and for each test an image is made of the location on the inner surface if the channel adjacent to the magnet, where the beads are expected to be trapped. By examining the images to see whether beads were trapped by the magnet, and how many beads were trapped, a suitable flow speed can be chosen to use for the assay, that will trap enough beads to produce a good signal without too much noise, but will wash away loose fluorescent molecules reasonably quickly. Once the calibration has been done, the same flow speed can be used for other assays, as long as the channel, magnet configuration and type and number of beads are still the same.

Optionally, there are two or more magnets located at different positions along the channel, for example a first magnet, and a second magnet located downstream from the first magnet, and at least some beads that fail to be trapped in the magnetic field adjacent to the first magnet are trapped in the magnetic field adjacent to the second magnet. Optionally, there are one or more additional magnets further downstream along the channel, that trap beads that failed to be trapped by the first two magnets. Optionally, the two or more magnets are close enough together, so that the different sets of trapped beads can be illuminated together by the excitation light, and their optical emission can be measured at the same time, for example by a camera with a field of view that includes all the sets of trapped beads.

The solution surrounding the beads, when they are prepared, often has loose reporter molecules dissolved in it, which will create a background level of the optical emission when the assay is done. Optionally, after the beads are trapped at the bottom of the channel, the fluid flowing past the beads is replaced by clean fluid, for example the same buffer solution, without reporter molecules dissolved in it, and this clean fluid washes away all or most of the loose reporter molecules in the vicinity of the trapped beads. As a result, the background level of optical emission may be greatly reduced, and may be so low that it can be ignored when measuring the optical emission, which has the potential advantage that there will be no need to measure and correct for the background optical emission.

At 2908, the trapped beads are illuminated with the excitation light, and at 2910, the resulting optical emission, for example fluorescent emission, is measured. The illumination system and emission detection and measurement system can have any of the configurations described above, and use any of the methods described above for exciting and detecting optical emission from magnetic beads, including, for example, the method of flowchart 2700 in FIG. 27. The method of flowchart 2900 may be especially suitable for use in a microfluidic cartridge, similar to that described above for FIG. 18, with microfluidic channel 2802 playing the role of detection chamber 1826 in FIG. 18, and with additional buffer solution from chamber 1808, or from an additional chamber, flowing past the beads trapped by the magnetic field in detection chamber 1826, to wash away loose reporter molecules, before the optical emission is measured.

At 2912, the concentration of target molecules in the sample is estimated from the measured optical emission from the trapped beads, for example making use of a calibration of the assay method performed using samples with known concentrations of target molecules. Alternatively, instead of or in addition to estimating the concentration of target molecules in the sample, a quantity of the target molecules in the sample is estimated, or the presence or absence of the target molecules in the sample is determined.

Optionally, after the assay is completed, the one or more magnets are removed from the vicinity of the channel, and more of the solution, or another liquid such as distilled water, is made to flow through the channel, washing away the beads that had been trapped by the magnetic field. The device can then be used for another assay. Even if the device is generally disposable, and not used for more than one assay of patients, a single device might still be used for multiple calibration assays, for example calibration assays used to find a suitable flow speed as described above.

It is expected that during the life of a patent maturing from this application many relevant assays, especially bioassays, using optical emission from magnetic beads, will be developed and the scope of the terms assay, bioassay, and optical emission is intended to include all such new technologies a priori.

As used herein the term “about” refers to +10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the Applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. (canceled)

2-62. (canceled)

63. An assay method for target molecules in a sample using optical emission from magnetic beads, comprising:
a) preparing the magnetic beads so, if excited, reporter molecules connected to the beads produce optical emission as a consequence of contact between the beads, the reporter molecules, and target molecules in the sample;
b) providing the prepared magnetic beads in a solution in a container, with one or more magnets producing a magnetic field inside the container that causes the beads to aggregate into a clump inside the container in less than 30 seconds;
c) exciting the optical emission from the reporter molecules connected to the magnetic beads in the clump; and
d) measuring the optical emission from the reporter molecules connected to the magnetic beads in the clump.

64. An assay method according to claim 63, wherein the one or more magnets producing the magnetic field are located beneath the container, and the one or more magnets have a magnetization oriented in a vertical direction.

65. An assay method according to claim 63, wherein the one or more magnets producing the magnetic field are located beneath the container, and the magnetic field causes the beads to aggregate into the clump at a location on a bottom surface of the inside of the container.

66. An assay method according to claim 65, wherein the solution has a depth of less than 4 mm above the location on the bottom surface where the clump aggregates.

67. An assay method according to claim 66, wherein a dot product of a magnetic field B produced by the magnet, and magnetic field gradient VB produced by the magnet, is greater than 0.05 T²/m over at least 50% of the solution in the container.

68. An assay method according to claim 66, wherein, for most of the magnetic beads, a travel time of the magnetic bead from anywhere in at least 50% of the solution in the container to a location at an inner surface of the container where the magnetic field is greatest, is less than 20 seconds, if the bead were to travel at an instantaneous velocity for which a magnetic force on the bead by the magnetic field balances a drag force on the bead in water at 20° C., at each location that the bead passes.

69. An assay method according to claim 63, wherein exciting the optical emission comprises illuminating the magnetic beads in the clump with an excitation light beam, and illuminating the magnetic beads in the clump comprises passing the excitation light beam through a first volume of the solution on the way to the clump, and measuring the optical emission from the magnetic beads in the clump comprises:
a) measuring an optical emission signal from the beads together with a background signal from the first volume;
b) illuminating a second volume of the solution without illuminating the clump, with the same or a similar excitation light beam, and measuring a background signal from the second volume; and
c) determining the optical emission from the magnetic beads, using the emission signal from the magnetic beads plus the background signal from the first volume, and the background signal from the second volume.

70. An assay method according to claim 63, wherein exciting the optical emission comprises illuminating the magnetic beads in the clump with an excitation light beam, the one or more magnets producing the magnetic field are located beneath the container, and the magnetic field causes the beads to aggregate into the clump at a location on a bottom surface of the inside of the container.

71. An assay method according to claim 70, comprising illuminating the location on the bottom surface with the excitation light beam, and measuring light coming from the location in a range of wavelengths of the optical emission, starting before the beads begin to aggregate into the clump, and wherein exciting the optical emission from the magnetic beads in the clump comprises illuminating the location with the excitation light beam while the beads are aggregating, and measuring the optical emission from the magnetic beads in the clump comprises measuring the emission from the location as a function of time while the beads are aggregating.

72. An assay method according to claim 71, comprising determining a concentration of target molecules in the sample from the measured emission by using the measurement of light coming from the location in the range of optical emission wavelengths to find a background level of the optical emission, and correcting the emission measured after the beads start aggregating for the background level.

73. An assay method according to claim 71, wherein measuring the emission as a function of time continues until the beads are fully aggregated into the clump, and determining the concentration of target molecules from the measured emission comprises using a level of emission measured when the beads are fully aggregated.

74. An assay method according to claim 73, comprising determining a concentration of target molecules in the sample from the measured emission using a rate of increase in the level of emission during at least one time period when the emission has not yet reached its maximum value.

75. An assay method according to claim 63, wherein the one or more magnets comprise at least one magnet that has a tip with a sharp point at its end in a direction of magnetization, and the magnet with a sharp tip is located outside the container, and the sharp tip is at a part of the magnet's surface that is closest to the container.

76. A method comprising performing the assay method of claim 63 a plurality of times successively using different samples, using different wells of a same well plate for the container each time, using the same one or more magnets each time, and positioning the well plate each time so that the well being used for that assay is over the one or more magnets when the assay is performed.

77. An assay method according to claim 63, wherein providing the prepared magnetic beads in the container comprises placing the magnetic beads into the container when the one or more magnets are already producing the magnetic field inside the container.

78. An assay method according to claim 63, wherein the beads form a clump adjacent to the one or more magnets within 10 seconds of an earliest time when the magnetic beads are in the solution in the container, and the one or more magnets are positioned to produce the magnetic field inside the container.

79. An assay method according to claim 63, wherein preparing the magnetic beads comprises:
a) entering the sample into a microfluidics cartridge;
b) exposing the sample, inside an incubation chamber of the cartridge, to probe molecules that selectively bind to the target molecules;
c) binding at least a portion of each probe molecule that was bound to a target molecule, indirectly to a magnetic bead; and
d) processing the beads, inside the same or another incubation chamber of the cartridge, so that an optical emission from reporter molecules bound to the probe molecules or portions of probe molecules, and also bound to the beads, will depend on how many probe molecules were bound to the target molecules;
and wherein exciting the optical emission and measuring the optical emission are also done in a chamber of the cartridge.

80. An assay method according to claim 63, wherein the container comprises a channel with the one or more magnets adjacent to it, and providing the prepared magnetic beads in a solution comprises:
a) causing the solution with the beads suspended in it to flow through the channel past the adjacent one or more magnets at a slow enough speed so that the magnetic field traps the beads and causes them to aggregate into the clump on an inner surface of the channel; and
b) causing the solution to continue to flow through the channel past the clump after it has aggregated;
and exciting and measuring the optical emission comprise exciting and measuring the optical emission after the continuing flow of the solution past the aggregated clump has washed away most loose molecules in the solution that would otherwise produce a background level of the optical emission from a vicinity of the clump, and wherein the one or more magnets comprise at least a first magnet adjacent to a first location in the channel, and a second magnet adjacent to a second location further along the channel in the direction of flow of the solution, and at least some beads that fail to be trapped and aggregated into a clump in the first location by the first magnet are trapped by second magnet and aggregate into a second clump at the second location, and the method also comprises exciting the optical emission and measuring the optical emission from the magnetic beads in the clump, and from the second clump.

81. An assay method according to claim 63, wherein the one or more magnets produce a magnetic field inside the container that causes the beads to aggregate into a clump inside the container in less than 10 seconds.

82. A system for measuring an optical emission signal from a quantity of magnetic beads in an assay of target molecules in a sample, comprising:
a) a container configured for holding the magnetic beads in a volume of a solution;
b) one or more magnets adjacent to the container, the magnets producing a magnetic field with a field gradient in the volume of solution that attracts the beads to form a clump at a bottom inner surface of the container adjacent to at least one of the magnets;
c) detection optics and a light sensing device that receives and measures the optical emission signal from the clump of magnetic beads; and
d) a recording device that outputs and/or stores data of the optical emission signal;
wherein the one or more magnets are located below the container, and a dot product of the magnetic field and the field gradient, at a top of the volume of the solution directly above the one or more magnets is at least 0.05 teslas squared per meter.

83. A system according to claim 82, wherein, for M280 magnetic beads, a ratio of magnetic force, to gravitational force reduced by buoyant force of the solution, on said magnetic beads, is greater than 10 throughout the volume of solution.

84. A system according to claim 82, wherein the illumination optics comprises a light beam deflecting element configured to direct the light beam to illuminate the clump of beads passing through a first volume of the solution, or to pass through a second volume of the solution going to the side of the clump, and wherein the recording device outputs and/or stores data of a background optical signal received from the second volume of the solution when the beam is directed to the side of the clump, in addition to outputting and/or storing data of the optical emission signal when the beam is illuminating the beads through the first volume of the solution, and wherein the light sensing device comprises a camera that distinguishes light emitted from the clump and the first volume, from light emitted from the second volume, by sensing them on different pixels.

85. A system according to claim 82, wherein the one or more magnets are below and adjacent to a bottom of the container.

86. A system according to claim 85, wherein the container is one of a plurality of similar wells comprised in a well plate, each well configured for holding the magnetic beads in the solution, and each well configured, at a same time or at different times, for attracting the beads into a clump at the bottom of the well using a magnetic field gradient, and for detecting an optical emission from the clump at the bottom of each well.

87. A system according to claim 86, also comprising a motor or actuator configured for moving the well plate horizontally, relative to the one or more magnets, successively bringing different wells above and adjacent to the same one or more magnets.

88. A system according to claim 82, also comprising a microfluidics cartridge that comprises:
a) the container;
b) one or more chambers configured for stably storing the magnetic beads and one or more reagents used for performing the assay, before the assay is performed;
c) an input port for entering the sample into the cartridge; and
d) one or more incubation chambers, the same as or different from the container, configured for performing the assay on the sample after it has been entered into the cartridge, using the reagents and the magnetic beads.
